SEQUENCE LISTING (42 sequences)

SEQ ID NO: 1 - DNA, 25 bp, Artificial Sequence
  Synthetic
caccgacactcaatcgtgccctcac

SEQ ID NO: 2 - DNA, 25 bp, Artificial Sequence
  Synthetic
aaacgtgagggcacgattgagtgtc

SEQ ID NO: 3 - DNA, 24 bp, Artificial Sequence
  Synthetic
caccgattaggagggcgcttccgg

SEQ ID NO: 4 - DNA, 24 bp, Artificial Sequence
  Synthetic
aaacccggaagcgccctcctaatc

SEQ ID NO: 5 - DNA, 25 bp, Artificial Sequence
  Synthetic
caccgagaacggcgatggccgccgt

SEQ ID NO: 6 - DNA, 25 bp, Artificial Sequence
  Synthetic
aaacacggcggccatcgccgttctc

SEQ ID NO: 7 - DNA, 24 bp, Artificial Sequence
  Synthetic
caccgcgccgtgtggtacagcccc

SEQ ID NO: 8 - DNA, 24 bp, Artificial Sequence
  Synthetic
aaacggggctgtaccacacggcgc

SEQ ID NO: 9 - DNA, 25 bp, Artificial Sequence
  Synthetic
caccgcgtgctctcggcgtgatcga

SEQ ID NO: 10 - DNA, 25 bp, Artificial Sequence
  Synthetic
aaactcgatcacgccgagagcacgc

SEQ ID NO: 11 - DNA, 25 bp, Artificial Sequence
  Synthetic
caccgaacgattcttcccgacgagg

SEQ ID NO: 12 - DNA, 24 bp, Artificial Sequence
  Synthetic
aaccctcgtcgggaagaatcgttc

SEQ ID NO: 13 - DNA, 25 bp, Artificial Sequence
  Synthetic
caccgtacttcaaggaatatcacgt

SEQ ID NO: 14 - DNA, 25 bp, Artificial Sequence
  Synthetic
aaacacgtgatattccttgaagtac

SEQ ID NO: 15 - DNA, 20 bp, Artificial Sequence
  Synthetic
tcagattgcagcgggatgtt

SEQ ID NO: 16 - DNA, 20 bp, Artificial Sequence
  Synthetic
gctgttaaaaaccggcctcc

SEQ ID NO: 17 - DNA, 20 bp, Artificial Sequence
  Synthetic
cctatcccagtgttccaccc

SEQ ID NO: 18 - DNA, 20 bp, Artificial Sequence
  Synthetic
gagaccacaggtgcagtcag

SEQ ID NO: 19 - DNA, 21 bp, Artificial Sequence
  Synthetic
tggcctgtgggacttaaatgg

SEQ ID NO: 20 - DNA, 20 bp, Artificial Sequence
  Synthetic
cgagtagctgcgactacagg

SEQ ID NO: 21 - DNA, 19 bp, Artificial Sequence
  Synthetic
ctgcagctcgtaaggggag

SEQ ID NO: 22 - DNA, 20 bp, Artificial Sequence
  Synthetic
ggggtgagcctatagtccca

SEQ ID NO: 23 - DNA, 20 bp, Artificial Sequence
  Synthetic
ttctcgagttcccaggagct

SEQ ID NO: 24 - DNA, 20 bp, Artificial Sequence
  Synthetic
ttggcatgatacccagtggc

SEQ ID NO: 25 - DNA, 18 bp, Artificial Sequence
  Synthetic
ctcccctcccactcgtca

SEQ ID NO: 26 - DNA, 19 bp, Artificial Sequence
  Synthetic
cctccctggagctcactct

SEQ ID NO: 27 - DNA, 20 bp, Artificial Sequence
  Synthetic
cccacattgaaacatgggcc

SEQ ID NO: 28 - DNA, 22 bp, Artificial Sequence
  Synthetic
tggtgactccatcaagcctatg

SEQ ID NO: 29 - DNA, 21 bp, Artificial Sequence
  Synthetic
tgcccttgtcttgtagtttcc

SEQ ID NO: 30 - DNA, 19 bp, Artificial Sequence
  Synthetic
cctgactgtgggcttgtat

SEQ ID NO: 31 - DNA, 20 bp, Artificial Sequence
  Synthetic
gcggtgagttcaggcttttt

SEQ ID NO: 32 - DNA, 20 bp, Artificial Sequence
  Synthetic
taatacgactcactataggg

SEQ ID NO: 33 - DNA, 23 bp, Artificial Sequence
  Synthetic
agccacgtggtctgggccttgca

SEQ ID NO: 34 - DNA, 32 bp, Artificial Sequence
  Synthetic
gagaagtcaactactacaagtttatcacctgc

SEQ ID NO: 35 - DNA, 25 bp, Artificial Sequence
  Synthetic
gtgtgggccttccaaaatgatcaag

SEQ ID NO: 36 - DNA, 18 bp, Artificial Sequence
  Synthetic
cacgtggctgagaagtca

SEQ ID NO: 37 - DNA, 23 bp, Artificial Sequence
  Synthetic
tgtggatgactctcaggatgttg

SEQ ID NO: 38 - DNA, 20 bp, Artificial Sequence
  Synthetic
tctacttcaaggaatatcac

SEQ ID NO: 39 - DNA, 24 bp, Artificial Sequence
  Synthetic
tgcttcagagtctgaagtcaaatg

SEQ ID NO: 40 - DNA, 18 bp, Artificial Sequence
  Synthetic
acatcctgacagtcatcc

SEQ ID NO: 41 - DNA, 25 bp, Artificial Sequence
  Synthetic
tgaagtcaaatccatgccaacattc

SEQ ID NO: 42 - DNA, 18 bp, Artificial Sequence
  Synthetic
cactctgaagcaacatcc

(19) United States
(12) Patent Application Publication
(10) Pub. No.: US 2022/0089602 A1
(43) Pub. Date: Mar. 24, 2022

(54) COMPOUNDS THAT INDUCE FERROPTIC CELL DEATH

(71) Applicant: The Board of Trustees of the University of Illinois, Urbana, IL (US)

(72) Inventors: Paul J. Hergenrother, Champaign, IL (US); Evijola Llabani, Chicago, IL (US)

(73) Assignee: The Board of Trustees of the University of Illinois, Urbana, IL (US)

(21) Appl. No.: 17/494,110

(22) Filed: Oct. 5, 2021

Related U.S. Application Data
(63) Continuation of application No. PCT/US2020/026873, filed on Apr. 6, 2020.
(60) Provisional application No. 62/830,384, filed on Apr. 6, 2019.

(51) Int. Cl. C07D 487/04; A61P 35/00; C07D 521/00

(52) U.S. Cl. CPC C07D 487/04; C07D 521/00; A61P 35/00

(57) ABSTRACT

The diterpene natural product pleuromutilin was subjected to reaction sequences focused on creating ring system diversity in few synthetic steps. This effort resulted in a collection of compounds with previously unreported ring systems, providing a novel set of structurally diverse and highly complex compounds suitable for screening in a variety of different settings. Biological evaluation identified the novel compound ferroptocide, a small molecule that rapidly and robustly induces ferroptotic death of cancer cells. Target identification efforts and CRISPR knockout studies reveal that ferroptocide is an inhibitor of thioredoxin, a key component of the antioxidant system in the cell. Ferroptocide positively modulates the immune system in a murine model of breast cancer and will be a useful tool to study the utility of pro-ferroptotic agents for treatment of cancer.

### RELATED APPLICATIONS

This application is a continuation under 35 U.S.C. 111(a) of International Application No. PCT/US2020/026873 filed Apr. 6, 2020, and published in English as WO 2020/210158 on Oct. 15, 2020, which claims priority from U.S. Provisional Application No. 62/830,384 filed Apr. 6, 2019, which applications and publication are incorporated herein by reference.

### GOVERNMENT SUPPORT

This invention was made with government support under Grant No. GM118575 awarded by the National Institutes of Health. The government has certain rights in the invention.

### BACKGROUND OF THE INVENTION

Structurally complex small molecules play an important role in probing biological systems and combating disease. Such compounds often contain dense polycyclic ring systems, multiple stereogenic centers, and spatially defined arrangements of functional groups. The complexity and three-dimensionality of these molecules allows for specific interactions with biological macromolecules and selective modulation of cellular pathways; such compounds are complimentary to those in large commercial screening collections that tend to have fewer stereogenic centers and more sp²-hybridized carbons.

Several strategies for the rapid and efficient synthesis of value-added complex compounds have been developed, including those that construct complex scaffolds from diverse collections of simple building blocks, and those that begin with complexity and build in diversity. For this later approach, natural products offer a rich and varied source of starting materials, and collections of compounds have been assembled using the Complexity-to-Diversity (CtD) strategy from the natural products adrenosterone, gibberellic acid, quinine, abietic acid, sinomenine, lycorine, yohimbine, haemanthamine, nitrogenous steroids of dutasteride and abiraterone acetate, ilimaquinone, and others. The resulting collections have been used to discover small molecules with anticancer and antimicrobial activities, autophagy inhibitors, and to identify predictive guidelines for broad-spectrum antibiotic discovery.

The problem is greater chemical structural diversity is needed for the discovery of new therapeutic agents and for elucidating the underlying mechanisms which can be targeted by other new therapeutic agents.

### SUMMARY

This disclosure provides application of the Complexity-to-Diversity (CtD) strategy to the natural product of pleuromutilin that provided a set of 29 structurally diverse and highly complex compounds. This set was then subjected to a phenotypic screen that allowed discovery of ferroptocide, which induces rapid ferroptotic death in immortalized cancer cell lines and primary cancer cells from patients. Cell culture studies demonstrate that ferroptocide-treated cells generate ROS and lipid peroxidation that result in inevitable cell death, an effect that can be prevented by pretreatment with known inhibitors of ferroptosis (trolox, ferrostatin-1, and DFO). Depletion of the glutathione antioxidant system and pharmacological inhibition or degradation of glutathione peroxidase 4 (GPX4) are the main known systems that control ferroptosis. In contrast, the collected data indicate that ferroptocide targets a different antioxidant system (thioredoxin) to induce ferroptosis; it is likely that inhibition of thioredoxin causes a drastic imbalance in the ROS levels, overwhelms cellular antioxidant responses (as seen at the transcript level), and causes ferroptosis. This hypothesis is supported by genetic knockdown studies of thioredoxin, which lead to accumulation of large amounts of ROS, lipid ROS and sensitization of siTXN cells to ferroptocide treatment.

Accordingly, this disclosure provides a compound of Formula I:

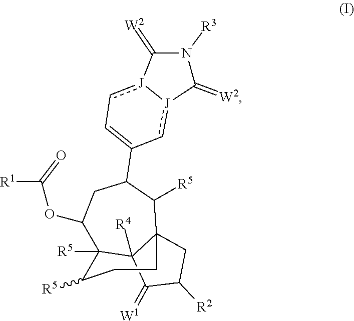

or a stereoisomer or salt thereof; wherein

J is CH or N and [symbol] is a single bond, or C and [symbol] is a double bond;

R¹ is halo, OH, —(C₁-C₆)alkyl-X, —(C₂-C₆)alkenyl-X, or heteroaryl, wherein the (C₁-C₆)alkyl moiety of —(C₁-C₆)alkyl-X is substituted optionally with halo;

X is absent, halo, OH, or —O(C═O)CH₃;

R² and R⁴ are independently H, halo, or OR^{A} wherein R^{A} is H, —(C₁-C₆)alkyl, or —(C═O)CH₃;

R³ is aryl, heteroaryl, heterocycloalkyl, —(C₃-C₆)cycloalkyl, —(C₂-C₆)alkynyl, or a group comprising a fluorescent tag, wherein aryl or heteroaryl is substituted optionally with halo, OH or —(C₁-C₆)alkyl;

each R⁵ is independently H or —(C₁-C₆)alkyl;

W¹ is absent, O or S; and each W² is independently absent, O or S.

Also, this disclosure provides a composition comprising a compound described herein and a pharmaceutically acceptable buffer, carrier, diluent, or excipient.

Additionally, this disclosure provides a method for inducing ferroptosis in cancer cells comprising contacting a cancer cell with an effective amount of a compound or composition described herein, thereby inducing ferroptosis.

This disclosure also provides a method for treating cancer in a cancer subject comprising administering an effective amount of a compound or composition described herein to the cancer subject in need of cancer treatment wherein the cancer is thereby treated.

The invention provides novel compounds of Formulas I-IV, intermediates for the synthesis of compounds of Formulas I-IV, as well as methods of preparing compounds of Formulas I-IV. The invention also provides compounds of Formulas I-IV that are useful as intermediates for the synthesis of other useful compounds. The invention provides for the use of compounds of Formulas I-IV for the manufacture of medicaments useful for the treatment of cancer in a mammal, such as a human.

The invention provides for the use of the compositions described herein for use in medical therapy. The medical therapy can be treating cancer, for example, breast cancer, lung cancer, pancreatic cancer, prostate cancer, or colon cancer. The invention also provides for the use of a composition as described herein for the manufacture of a medicament to treat a disease in a mammal, for example, cancer in a human. The medicament can include a pharmaceutically acceptable diluent, excipient, or carrier.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Ferroptocide displays broad activity in a 72 hr cell viability assay in immortalized cancer cells and in primary cells isolated from metastatic cancer patients. PPC: primary peritoneal carcinoma. Data represent the mean±s.e.m. of biological replicates, n≥3.

FIG. 2. Tool compounds P28, P29, and P30 retain biological activity in a 72 hr cell viability assay in ES-2 cells. Confocal microscopy images of ES-2 cells treated with fluorescent analogue, P30 (1 M) for 15 min show non-nuclear localization (green). Nucleus was stained with Hoechst (blue).

FIG. 3. Ferroptocide induces rapid non-apoptotic cell death. a. Speed of death of cells treated with ferroptocide versus 16 other anticancer compounds in ES-2 cells (all tested at 10 μM). Cell viability was assessed by AV/PI analysis. Data is representative of three biological replicates. b. Time-course analysis of ES-2 cell viability upon treatment with ferroptocide (10 μM) indicates a non-apoptotic mode of cell death. AV/PI graphs are representative of three biological replicates. c. Effect of pre-treatment with Q-VD-OPh (25 μM) for 2 hr followed by dose-response treatment with ferroptocide or positive control Raptinal (5 μM) for 13 hr in ES-2 cells. Data are plotted as the mean±s.e.m., n=3 biological replicates. *** 0.0001≤p<0.001, n.s. p>0.05. d. Transmission electron micrographs of ES-2 cells treated with DMSO (left), ferroptocide (10 μM, center) or staurosporine (STS, 10 μM, right) for 30 min. The images show lack of apoptotic morphological features and swelling of mitochondria upon ferroptocide treatment (arrows) versus controls. TEM data are representative images. e. Co-localization analysis with mitochondria. ES-2 cells were stained with MitoTracker Red (100 nM) followed by 30 min treatment with fluorescent analogue P30 (10 μM). Nucleus was stained with Hoechst. Yellow dots indicate P30 (green) on the mitochondria (red) in merged images. f. Ferroptocide induces dose-dependent ROS generation within 1 hr similar to positive control TBHP in ES-2 cells (and also in HCT 116 cells, see FIG. 8f). DMSO and etoposide were included as negative controls. Data are representative of three independent experiments.

FIG. 4. Ferroptocide kills cancer cells through ferroptosis. a. Ability of iron chelator deferoxamine (DFO) to prevent ferroptosis upon treatment with ferroptocide or positive control RSL3 for 1 hr in ES-2 cells (C11-BODIPY probe, lipid ROS). Data is representative of three independent experiments. b. Lipophilic antioxidant Trolox (250 μM) rescues ES-2 cells from ferroptocide-induced cytotoxicity after 14 hr incubation. c. Ability of ferroptosis inhibitor, ferrostatin (2 μM), to protect cells against ferroptocide treatment after 14 hr in ES-2 cells. d. Effect of DFO (100 μM) on viability of ES-2 cells after 14 hr incubation with ferroptocide and erastin (positive control). e. Comparison of speed of cell death of ferroptocide, RSL3, and erastin, each at (10 μM) in HCT 116 and A549 (two K-RAS mutant cancer cell lines) respectively. b-e. Cell viability was determined with AV/PI staining. Data are plotted as the mean±s.e.m., n=3 biological replicates. **** p<0.0001, *** 0.0001≤p<0.001, ** 0.001≤p<0.01, n.s. p>0.05.

FIG. 5. Ferroptocide selectively and covalently modifies its target in cells. a. Proteomic profile for fluorescent analogue P30 in HCT 116 cells after 60 min treatment reveals labeling of five main bands. (Note: Band A and A′ often appear as one band). Coomassie stain of gel demonstrates equal loading. b. Competitive profiling of the proteomic reactivity of P30 with ferroptocide. HCT 116 cells were pre-treated with DMSO or various concentrations of ferroptocide (30 min) followed by treatment with P30 (1 μM, 30 min) and analyzed by in-gel fluorescence assay. Specific competed proteins are marked as B and D. Coomassie stain of gel demonstrates equal loading. c. Ferroptocide covalently modifies the same target(s) in multiple cell lines. Competition experiments were performed by treatment of cells with DMSO or ferroptocide (20 μM, 30 min) followed by P30 incubation (1 μM, 30 min) and then analyzed using an in-gel fluorescence assay. Images are representative of three biological replicates. Coomassie stain of gels demonstrates equal loading. d. Ferroptocide causes the same proteomic competitive profile in primary cells isolated from metastatic cancer patient samples. Competition experiments were performed by treatment of cells with DMSO or ferroptocide (20 μM, 30 min) followed by P30 incubation (1 μM, 30 min) and then analyzed using an in-gel fluorescence assay. Representative images of two biological replicates. PPC: primary peritoneal carcinomatosis. Coomassie stain of gels demonstrates equal loading. e. Schematic of biotin-streptavidin pulldown method: Treatment of HCT 116 cells with ferroptocide (30 min) and P29 (60 min) was followed by CuAAC reaction with biotin-azide and enrichment with streptavidin magnetic beads. On-bead trypsin digestion coupled to LC/LC-MS/MS provided a list of over 300 targets. f. Enrichment of proteins based on p values <0.05 and fold change >3 in HCT 116 cells. Thioredoxin (TXN) was a top target candidate.

FIG. 6. Ferroptocide modulates active site cysteines of thioredoxin and has activity in vivo. a. Immunoblot of thioredoxin pulldown upon treatment of HCT 116 cells with DMSO or P29 (20 μM, 60 min) followed by CuAAC reaction with biotin-azide and enrichment with streptavidin magnetic beads. Thioredoxin appeared only in the P29-treated samples. BPD (biotin pulldown) and input (soluble cell lysate subjected to pulldown). Images are representative of three biological experiments. b. Effect of ferroptocide (20 μM) and known inhibitors PMX464 and PX-12 (50 μM) on thioredoxin activity in ES-2 cells after 30 min incubation. p-values are relative to DMSO control; ** 0.001≤p<0.01, * 0.01≤p<0.05, n.s. p>0.05. c. Competition profile of thioredoxin labeling by probe P29 (20 μM, 60 min) upon pre-treatment with DMSO or ferroptocide (20 μM, 30 min) followed by CuAAC with Cy3 azide in HCT 116 cells overexpressing TXN-GFP plasmid vs. non-transfected (wild type) cells, Cy3 channel. Red box indicates competition of the band of interest. Representative in-gel fluorescence images of n=3 biological replicates. Coomassie stain of gel demonstrates equal loading. d. Identification of ferroptocide labeling sites of thioredoxin. In-gel fluorescence scanning of HCT 116 cells overexpressing each thioredoxin-mutated cysteine plasmids. Cells were pre-treated with DMSO or ferroptocide (20 μM, 30 min) followed by incubation with P29 probe (5 μM, 60 min) and then CuAAC reaction with Cy3 azide. The serine mutations of the active site cysteines 32, 35 and cysteine 73 diminished compound labeling. Data are representative of three independent experiments. Coomassie stain of gels demonstrates equal loading. e. Crystal structure of thioredoxin with cysteine residues colored in red. f. Ferroptocide inhibits subcutaneous 4T1 tumor growth in immunocompetent Balb/c mice (left) but not in immunodeficient SCID mice (right) as measured by tumor volume. Ferroptocide was administered intraperitoneally at 50 mg/kg, twice a week, five doses (n=7 mice per group). Data represent the mean±s.e.m. p values are relative to vehicle control; ** p<0.01, * 0.01≤p<0.05.

FIG. 7. Complexity and bioactivity of P compound set. a. Comparison of complexity metrics (Fsp3, chiral centers, and ring complexity) of pleuromutilin-derived compounds with various small molecule compound libraries. Violin plots shown, where width represents the distribution while the blue dot and line represent the mean and standard deviation. b. Lack of hemolytic activity of P4 and ferroptocide in red blood cells, upon 2 hr treatment with 333 μM of each compound, positive control (MiliQ H2O) and negative control (DMSO in RBC buffer). Error is standard error of the mean, n=3. c. Promiscuous bioactivity of the iodo fluorescent analogue (P31) in HCT 116 cells upon 30 min dose-dependent treatment with compound. Coomassie stain of gel demonstrates equal loading. d. Effect of ferroptocide and approved and experimental chemotherapeutics (5-FU, Cisplatin, Etoposide, PAC-1) in primary patient-derived cells in a 72 hr cell viability Alamar Blue assay. Cells tested are those shown in right side of FIG. 1. Box-and-whisker plots: the bottom and top of the box present the first (Q1) and third quartile (Q3), respectively; the band inside the box is the median. Data falling outside Q1 and Q3 are plotted as outliers. e. ES-2 cells were pretreated with the three compounds shown (at 3× the IC50 value) for 30 min followed by treatment with fluorescent analogue P30 (5 μM). Nucleus was stained with Hoechst. P30 is competed by ferroptocide, but not by the other three a-chloro esters.

FIG. 8. Investigating the mode of action of ferroptocide. a-b. Speed of cell death induced by ferroptocide (10 μM) and other tool compounds (STS, MNNG) in Mia PaCa-2 and HCT 116 cells. Ferroptocide causes 50% cell death in 2 hr and 7 hr in each cell line, respectively. Cell viability was determined via AV/PI staining. Error is standard error of the mean, n≥3. c. Effect of pre-treatment with Q-VD-OPh (25 μM) for 2 hr followed by dose-response treatment of ferroptocide or positive control Raptinal (10 μM) for 13 hr in HCT 116 cells. Data are plotted as the mean±s.e.m., n=3 biological replicates. ** 0.001≤p<0.01, n.s. p>0.05. d. Immunoblots of ES-2 and HCT 116 cells indicate no PARP-1 cleavage after 1 hr and 7 hr treatment (respectively) with ferroptocide (P18). The positive control, Raptinal, induces PARP-1 cleavage in both cell lines. e. Co-localization analysis of BODIPY azide dye with mitochondria. ES-2 cells were stained with MitoTracker Red (100 nM) followed by 30 min treatment with BODIPY dye (1 μM). Nucleus was stained with Hoechst. f. Dose-dependent ROS generation upon ferroptocide treatment for 1.5 hr compared to the positive control TBHP and negative controls DMSO and etoposide in HCT 116 cells. g. Monitoring mitochondrial ROS levels in ES-2 cells after treatment with ferroptocide at the indicated concentrations for 1 hr using a MioSox Red (5 μM) probe. IB-DNQ and rotenone are used as positive controls. a-g. Data are representative of three independent experiments.

FIG. 9. Ferroptocide is a robust inducer of ferroptotic cell death. a. Ability of the iron chelator, deferoxamine (DFO) to protect HCT 116 cells from ferroptocide-induced lipid ROS within 2 hr. TBHP was used as a positive control upon 6 hr treatment. b. Ability of the iron chelator, deferoxamine (DFO) to protect 4T1 murine cells from ferroptocide-induced lipid ROS within 2 hr using the C11-Bodipy probe. RSL3 was used as a positive control upon 2 hr treatment. c. Ability of lipophilic antioxidant trolox (250 M) and N-acetyl cysteine (NAC-1, 5 mM) to protect against ferroptocide-induced cell death in HCT 116 cells (10 hr). Raptinal and DMSO were used as negative controls, TBHP was used as a positive control. d. Ability of ferrostatin (2 μM) to rescue cells from ferroptocide and RSL3-induced cell death, RSL3 positive control. e. Effect of ferroptosis inhibitor, deferoxamine (100 μM) on HCT 116 cell viability after 24 hr ferroptocide incubation. a-b Data are representative of n=3 biological replicates. c-e Data are plotted as the mean±s.d., n=3 biological triplicates. **** p<0.0001, *** 0.0001≤p<0.001, ** 0.001≤p<0.01, *0.01≤p<0.05, n.s. p>0.05. f. Ability of ferrostatin-1 (2 μM) and deferoxamine (DFO, 100 M) to rescue 4T1 cells from ferroptocide-induced cell death after 18 hr. g. Ability of trolox (250 μM), NAC-1 (5 mM), ferrostatin (2 μM) and DFO (100 μM) to rescue A549 cells from ferroptocide-induced cell death after 12 hr. f-g Data are plotted as the mean±s.d., n=3 biological triplicates. **** p<0.0001, *** 0.0001≤p<0.001, ** 0.001≤p<0.01, *0.01≤p<0.05, n.s. p>0.05. h. Generation of lipid ROS in HCT 116 cells upon 2 hr treatment using C11-bodipy probe, n=3 biological replicates. i. Treatment of ES-2 cells for 1 hr with ferroptocide (10 μM) does not cause direct GPX4 inhibition compared to RSL3 (10 M) in a phosphatidylcholine hydroperoxide (PCOOH) LC-MS based assay. Raptinal (10 μM) and DMSO were used as negative controls. Data are plotted as the mean±s.e.m., n=3. *0.01≤p<0.05, n.s. p>0.05 vs. PCOOH control. j. Modulation of 35/40 genes involved in ferroptosis upon 6 hr treatment of HT-29 cells with ferroptocide (10 μM) (FDR≤0.05). k. AnnexinV/PI graphs of HT-29 cells treated with ferroptocide (10 μM) for 6 hr. RNA of these cells was isolated and used for RNA seq data. 1. Upregulation of KEAP1-Nrf2 pathway in ferroptocide-treated HT-29 cells. m. Modulation of oxidative-stress pathways upon ferroptocide treatment, RNA-seq data of HT-29 cells.

FIG. 10. Investigating reactivity with thiols. a-b. Monitoring in vitro reactivity of ferroptocide (100 μM) and the iodo analogue, P23 (100 μM), with excess glutathione (5 mM) upon incubation at the indicated time points, in PBS buffer at 37° C., using an LC-MS-based method, respectively.

FIG. 11. Investigating the target(s) of ferroptocide. a. Proteomic profiling of probe P30 (1 μM, 30 min) upon pre-treatment with DMSO or ferroptocide (20 μM, 30 min) in HCT 116 cells after 72 hr siRNA transfection of GSTO1 and KEAP1 targets respectively. Western blot analysis of siRNA knockdown efficiency. Coomassie stain of gels demonstrates equal loading. b. In-gel fluorescence scanning of CRISPR Cas9-generated isogenic cell lines for six targets in HCT 116 cells treated with DMSO or ferroptocide (20 μM, 30 min) followed by 30 min incubation with probe P30 (1 μM) and separation of proteins via SDS-PAGE gel. Coomassie stain of gels demonstrates equal loading. c. Comparing in vitro activity of ferroptocide and known thioredoxin inhibitors, PX-12 and PMX464 to inhibit purified human thioredoxin in a dose-dependent manner after 30 min treatment using a thioredoxin activity kit. Data are plotted as the mean±s.d., n=3 biologically independent samples. p values are relative to DMSO control, **** p<0.0001, **0.001≤p<0.01, * 0.01≤p<0.05. n.s. p>0.05 d. In-gel fluorescence scanning of HCT 116 cells overexpressing TXN-GFP shows a new band (red arrow) at 37 kDa, wild type are non-transfected cells, GFP channel. Coomassie stain of gel demonstrates equal loading. e. Assessing transfection efficiency of HCT 116 cells overexpressing C32S, C35S, empty GFP vector, C62S, C69S, and C73S mutants. WT are non-transfected HCT 116 cells. a-e. Data are representative of n=3, biological triplicates.

FIG. 12. Linking thioredoxin to ferroptosis. a. Genetic knockdown of thioredoxin leads to ROS and lipid ROS generation in HCT 116 cells after 72 hr transfection. siGAPDH and siNeg serve as negative controls. Data are representative of three independent experiments. b. Western blot analysis of siRNA knockdown efficiency for samples in a and c. c. Treatment of HCT 116 cells with trolox (250 μM), deferoxamine (100 μM), and ferrostatin-1 (2 μM) for 2 hr did not rescue them from the effect of thioredoxin siRNA after 72 hr transfection. d-e. Monitoring the ability of ferroptosis inhibitors to rescue cell death-induced from thioredoxin inhibitors (PMX464, PX-12), negative control raptinal and positive control RSL3 in ES-2 (14 hr) and A549 (24 hr) cells respectively. f. siRNA of thioredoxin in HCT 116 cells (48 hr) sensitizes them to ferroptocide treatment (10 μM) but not raptinal at the indicated time points. g. Assessing the transfection efficiency of thioredoxin knockdown in the time course studies (f). c-f. Cell viability was determined with AV/PI staining. Data are plotted as the mean±s.e.m., n=3 biological replicates.**** p<0.0001, *** 0.0001≤p<0.001, ** 0.001≤p<0.01, n.s. p>0.05.

FIG. 13. Ferroptocide pharmacokinetics C57BL/6 mice were treated with ferroptocide (40 mg/kg) via i.p. injection. Points: mean (n=3), bars: standard error.

FIG. 14. Time-course of ES-2 cells upon treatment with ferroptocide and 16 toxins (all tested at 10 μM) corresponding to FIG. 3a. Cell viability was determined via AV/PI analysis. Data is represented as mean±s.e.m., n=3 biological replicates.

### DETAILED DESCRIPTION

The chemical diversification of natural products provides a robust and general method for creation of stereochemically rich and structurally diverse small molecules. The resulting compounds have physicochemical traits different from those in most screening collections, and as such are an excellent source for biological discovery.

The CtD strategy is applied to the natural product pleuromutilin (P) with an emphasis on transforming the highly dense ring system of P into compounds with novel and complex ring architectures in short synthetic sequences (Scheme A). The resulting compounds were then evaluated for their ability to induce rapid death of cancer cells, with an eye toward discovery of compounds with unusual modes of action. We now report the identification of ferroptocide, a novel compound that induces rapid ferroptotic death of cancer cells and inhibits thioredoxin; its mechanism of ferroptotic induction makes ferroptocide distinct from and complementary to the existing ferroptosis inducers. Additionally, ferroptocide has immunostimulatory activity in a murine cancer model and thus will be an important tool for further investigating the potential of ferroptosis-inducing agents to act in concert with the immune system as an anticancer strategy.

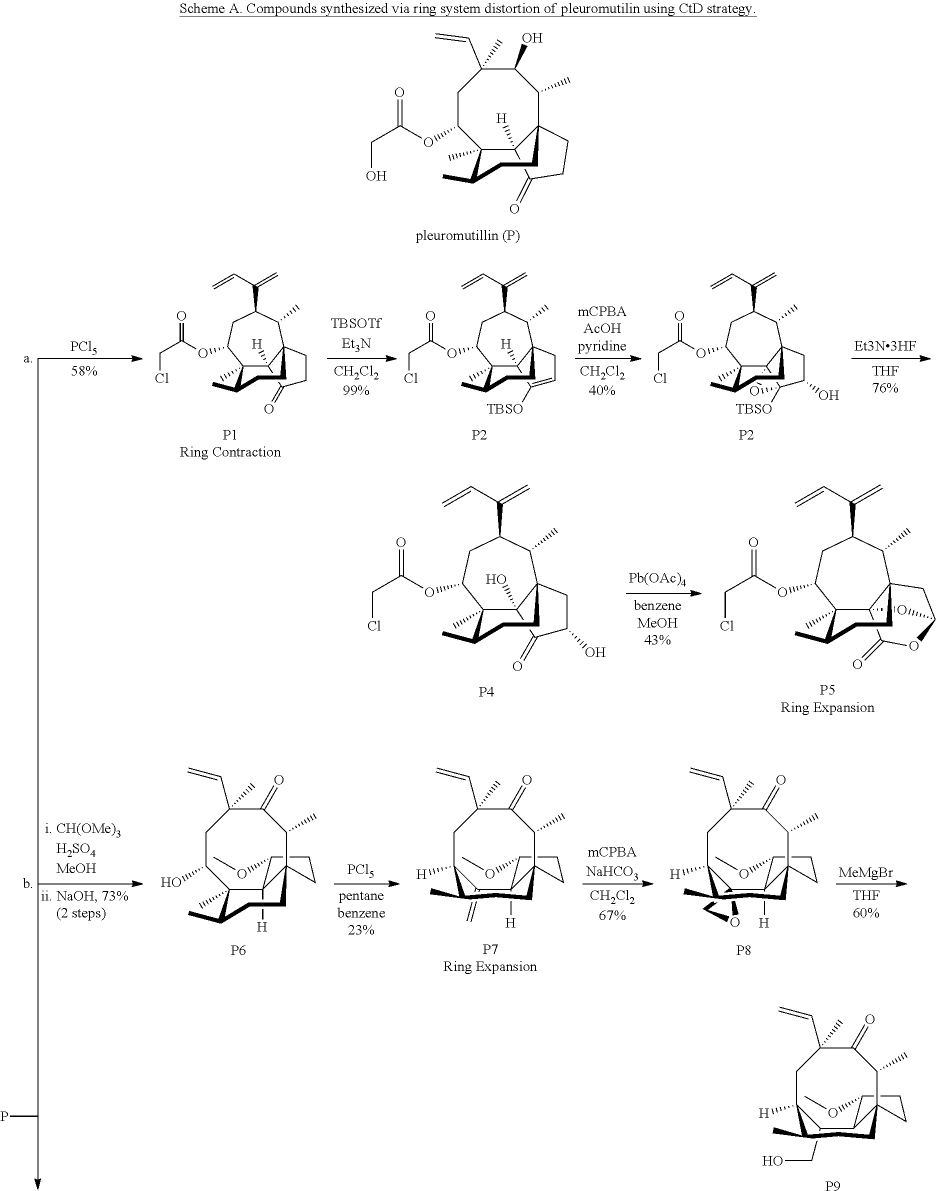

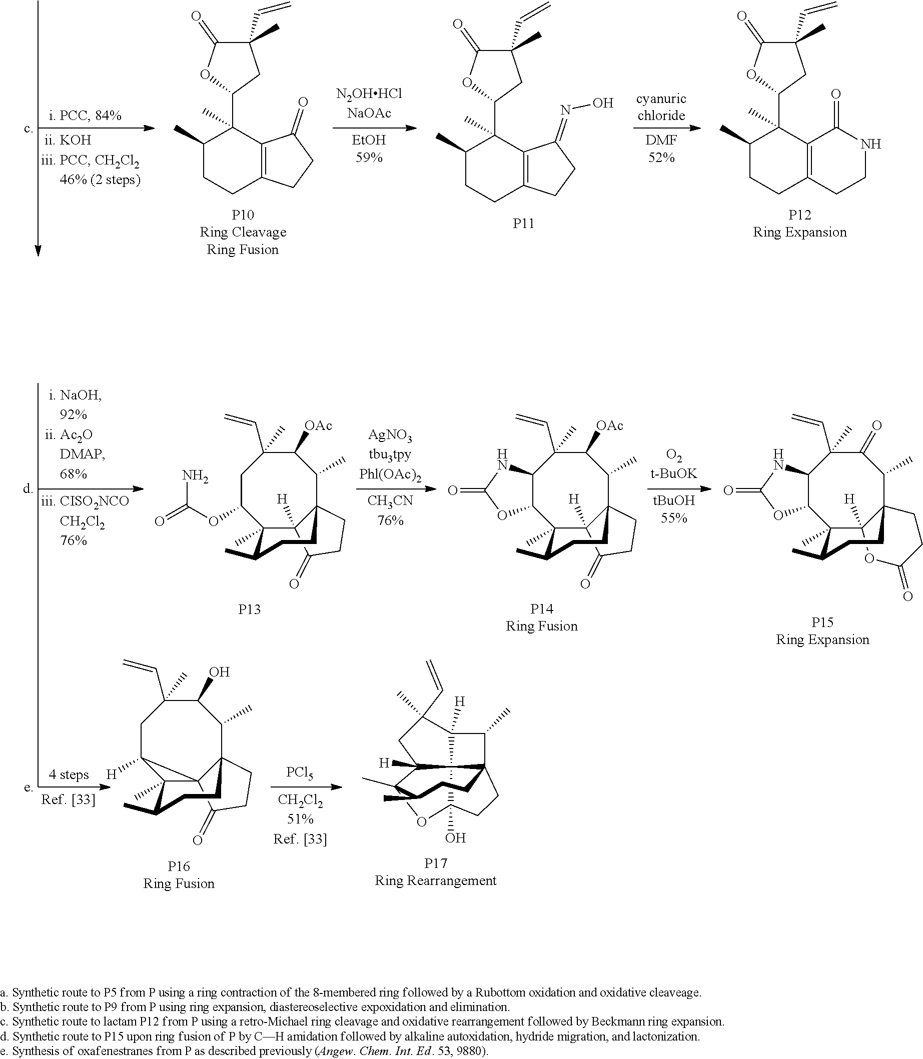

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and sub-combinations possible of the group are intended to be individually included in the disclosure. When a compound is described herein such that a particular isomer, enantiomer or diastereomer of the compound is not specified, for example, in a formula or in a chemical name, that description is intended to include each isomer and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

As used herein, the terms “providing”, “administering,” “introducing,” are used interchangeably herein and refer to the placement of the compositions of the disclosure into a subject by a method or route which results in at least partial localization of the composition to a desired site. The compositions can be administered by any appropriate route which results in delivery to a desired location in the subject.

The compositions described herein may be administered with additional compositions to prolong stability and activity of the compositions, or in combination with other therapeutic drugs.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein suitably may be practiced in the absence of any element or elements, limitation or limitations which is not specifically disclosed herein.

This disclosure provides methods of making the compounds and compositions of the invention. The compounds and compositions can be prepared by any of the applicable techniques described herein, optionally in combination with standard techniques of organic synthesis. Many techniques such as etherification and esterification are well known in the art. However, many of these techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6; as well as standard organic reference texts such as March's Advanced Organic Chemistry: Reactions, Mechanisms, and Structure, 5th Ed., by M. B. Smith and J. March (John Wiley & Sons, New York, 2001); Comprehensive Organic Synthesis. Selectivity, Strategy & Efficiency in Modern Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983); for heterocyclic synthesis see Hermanson, Greg T., Bioconjugate Techniques, Third Edition, Academic Press, 2013.

The formulas and compounds described herein can be modified using protecting groups. Suitable amino and carboxy protecting groups are known to those skilled in the art (see for example, Protecting Groups in Organic Synthesis, Second Edition, Greene, T. W., and Wutz, P. G. M., John Wiley & Sons, New York, and references cited therein; Philip J. Kocienski; Protecting Groups (Georg Thieme Verlag Stuttgart, New York, 1994), and references cited therein); and Comprehensive Organic Transformations, Larock, R. C., Second Edition, John Wiley & Sons, New York (1999), and referenced cited therein.

As used herein, the term “substituted” or “substituent” is intended to indicate that one or more (for example, 1-20 in various embodiments, 1-10 in other embodiments, 1, 2, 3, 4, or 5; in some embodiments 1, 2, or 3; and in other embodiments 1 or 2) hydrogens on the group indicated in the expression using “substituted” (or “substituent”) is replaced with a selection from the indicated group(s), or with a suitable group known to those of skill in the art, provided that the indicated atom's normal valency is not exceeded, and that the substitution results in a stable compound. Suitable indicated groups include, e.g., alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, and cyano. Additionally, non-limiting examples of substituents that can be bonded to a substituted carbon (or other) atom include F, Cl, Br, I, OR′, OC(O)N(R′)₂, CN, CF₃, OCF₃, R′, O, S, C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂NHC(O)R′, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further substituted. When a substituent is monovalent, such as, for example, F or Cl, it is bonded to the atom it is substituting by a single bond. When a substituent is more than monovalent, such as O, which is divalent, it can be bonded to the atom it is substituting by more than one bond, i.e., a divalent substituent is bonded by a double bond; for example, a C substituted with O forms a carbonyl group, C═O, wherein the C and the O are double bonded. Alternatively, a divalent substituent such as O, S, C(O), S(O), or S(O)₂ can be connected by two single bonds to two different carbon atoms. For example, O, a divalent substituent, can be bonded to each of two adjacent carbon atoms to provide an epoxide group, or the O can form a bridging ether group between adjacent or non-adjacent carbon atoms, for example bridging the 1,4-carbons of a cyclohexyl group to form a [2.2.1]-oxabicyclo system. Further, any substituent can be bonded to a carbon or other atom by a linker, such as (CH₂)ₙ or (CR′₂)ₙ wherein n is 1, 2, 3, or more, and each R′ is independently selected.

The term “halo” or “halide” refers to fluoro, chloro, bromo, or iodo. Similarly, the term “halogen” refers to fluorine, chlorine, bromine, and iodine.

The term “alkyl” refers to a branched or unbranched hydrocarbon having, for example, from 1-20 carbon atoms, and often 1-12, 1-10, 1-8, 1-6, or 1-4 carbon atoms; or for example, a range between 1-20 carbon atoms, such as 2-6, 3-6, 2-8, or 3-8 carbon atoms. As used herein, the term “alkyl” also encompasses a “cycloalkyl”, defined below. Examples include, but are not limited to, methyl, ethyl, 1-propyl, 2-propyl (iso-propyl), 1-butyl, 2-methyl-1-propyl (isobutyl), 2-butyl (sec-butyl), 2-methyl-2-propyl (t-butyl), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, hexyl, octyl, decyl, dodecyl, and the like. The alkyl can be unsubstituted or substituted, for example, with a substituent described above. The alkyl can also be optionally partially or fully unsaturated. As such, the recitation of an alkyl group optionally includes a carbon chain moiety that is an alkenyl or alkynyl group. The alkyl can be a monovalent hydrocarbon radical, as described and exemplified above, or it can be a divalent hydrocarbon radical (i.e., an alkylene).

An alkylene is an alkyl group having two free valences at a carbon atom or two different carbon atoms. Similarly, alkenylene and alkynylene are respectively an alkene and an alkyne having two free valences at two different carbon atoms.

The term “cycloalkyl” refers to cyclic alkyl groups of, for example, from 3 to 10 carbon atoms having a single cyclic ring or multiple condensed rings. Cycloalkyl groups include, by way of example, single ring structures such as cyclopropyl, cyclobutyl, cyclopentyl, cyclooctyl, and the like, or multiple ring structures such as adamantyl, and the like. The cycloalkyl can be unsubstituted or substituted. The cycloalkyl group can be monovalent or divalent, and can be optionally substituted as described for alkyl groups. The cycloalkyl group can optionally include one or more cites of unsaturation, for example, the cycloalkyl group can include one or more carbon-carbon double bonds, such as, for example, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, and the like.

The term “heterocycloalkyl” refers to a saturated or partially saturated monocyclic, bicyclic, or polycyclic ring containing at least one heteroatom selected from nitrogen, sulfur, oxygen, preferably from 1 to 3 heteroatoms in at least one ring. Each ring is preferably from 3 to 10 membered, more preferably 4 to 7 membered. Examples of suitable heterocycloalkyl substituents include pyrrolidyl, tetrahydrofuryl, tetrahydrothiofuranyl, piperidyl, piperazyl, tetrahydropyranyl, morpholino, 1,3-diazapane, 1,4-diazapane, 1,4-oxazepane, and 1,4-oxathiapane. The group may be a terminal group or a bridging group.

The term “aryl” refers to an aromatic hydrocarbon group derived from the removal of at least one hydrogen atom from a single carbon atom of a parent aromatic ring system. The radical attachment site can be at a saturated or unsaturated carbon atom of the parent ring system. The aryl group can have from 6 to 30 carbon atoms, for example, about 6-10 carbon atoms. In other embodiments, the aryl group can have 6 to 60 carbons atoms, 6 to 120 carbon atoms, or 6 to 240 carbon atoms. The aryl group can have a single ring (e.g., phenyl) or multiple condensed (fused) rings, wherein at least one ring is aromatic (e.g., naphthyl, dihydrophenanthrenyl, fluorenyl, or anthryl). Typical aryl groups include, but are not limited to, radicals derived from benzene, naphthalene, anthracene, biphenyl, and the like. The aryl can be unsubstituted or optionally substituted.

The term “heteroaryl” refers to a monocyclic, bicyclic, or tricyclic ring system containing one, two, or three aromatic rings and containing at least one nitrogen, oxygen, or sulfur atom in an aromatic ring. The heteroaryl can be unsubstituted or substituted, for example, with one or more, and in particular one to three, substituents, as described in the definition of “substituted”. Typical heteroaryl groups contain 2-20 carbon atoms in the ring skeleton in addition to the one or more heteroatoms. Examples of heteroaryl groups include, but are not limited to, 2H-pyrrolyl, 3H-indolyl, 4H-quinolizinyl, acridinyl, benzo[b]thienyl, benzothiazolyl, β-carbolinyl, carbazolyl, chromenyl, cinnolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrrolyl, quinazolinyl, quinolyl, quinoxalinyl, thiadiazolyl, thianthrenyl, thiazolyl, thienyl, triazolyl, tetrazolyl, and xanthenyl. In one embodiment the term “heteroaryl” denotes a monocyclic aromatic ring containing five or six ring atoms containing carbon and 1, 2, 3, or 4 heteroatoms independently selected from non-peroxide oxygen, sulfur, and N(Z) wherein Z is absent or is H, O, alkyl, aryl, or (C₁-C₆)alkylaryl. In some embodiments, heteroaryl denotes an ortho-fused bicyclic heterocycle of about eight to ten ring atoms derived therefrom, particularly a benz-derivative or one derived by fusing a propylene, trimethylene, or tetramethylene diradical thereto.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof, such as racemic mixtures, which form part of the present invention. Many organic compounds exist in optically active forms, i.e., they have the ability to rotate the plane of plane-polarized light. In describing an optically active compound, the prefixes D and L, or R and S. are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate (defined below), which may occur where there has been no stereoselection or stereospecificity in a chemical reaction or process.

A coding sequence is the part of a gene or cDNA that codes for the amino acid sequence of a protein, or for a functional RNA such as a tRNA or rRNA. Complement or complementary sequence means a sequence of nucleotides which forms a hydrogen-bonded duplex with another sequence of nucleotides according to Watson-Crick base-pairing rules. Downstream refers to a relative position in DNA or RNA and is the region towards the 3′ end of a strand.

Expression refers to the transcription of a gene into structural RNA (rRNA, tRNA) or messenger RNA (mRNA) and subsequent translation of an mRNA into a protein.

An amino acid sequence that is functionally equivalent to a specifically exemplified TCR sequence is an amino acid sequence that has been modified by single or multiple amino acid substitutions, by addition and/or deletion of amino acids, or where one or more amino acids have been chemically modified, but which nevertheless retains the binding specificity and high affinity binding activity of a cell-bound or a soluble TCR protein of the present disclosure. Functionally equivalent nucleotide sequences are those that encode polypeptides having substantially the same biological activity as a specifically exemplified cell-bound or soluble TCR protein. In the context of the present disclosure, a soluble TCR protein lacks the portions of a native cell-bound TCR and is stable in solution (i.e., it does not generally aggregate in solution when handled as described herein and under standard conditions for protein solutions).

Two nucleic acid sequences are heterologous to one another if the sequences are derived from separate organisms, whether or not such organisms are of different species, as long as the sequences do not naturally occur together in the same arrangement in the same organism.

Homology refers to the extent of identity between two nucleotide or amino acid sequences.

Isolated means altered by the hand of man from the natural state. If an “isolated” composition or substance occurs in nature, it has been changed or removed from its original environment, or both. For example, a polynucleotide or a polypeptide naturally present in a living animal is not isolated, but the same polynucleotide or polypeptide separated from the coexisting materials of its natural state is isolated, as the term is employed herein.

A nucleic acid construct is a nucleic acid molecule which is isolated from a naturally occurring gene or which has been modified to contain segments of nucleic acid which are combined and juxtaposed in a manner which would not otherwise exist in nature.

Nucleic acid molecule means a single- or double-stranded linear polynucleotide containing either deoxyribonucleotides or ribonucleotides that are linked by 3′-5′-phosphodiester bonds.

Two DNA sequences are operably linked if the nature of the linkage does not interfere with the ability of the sequences to affect their normal functions relative to each other. For instance, a promoter region would be operably linked to a coding sequence if the promoter were capable of effecting transcription of that coding sequence.

A polypeptide is a linear polymer of amino acids that are linked by peptide bonds.

Promoter means a cis-acting DNA sequence, generally 80-120 base pairs long and located upstream of the initiation site of a gene, to which RNA polymerase may bind and initiate correct transcription. There can be associated additional transcription regulatory sequences which provide on/off regulation of transcription and/or which enhance (increase) expression of the downstream coding sequence.

A recombinant nucleic acid molecule, for instance a recombinant DNA molecule, is a novel nucleic acid sequence formed in vitro through the ligation of two or more nonhomologous DNA molecules (for example a recombinant plasmid containing one or more inserts of foreign DNA cloned into at least one cloning site).

Transformation means the directed modification of the genome of a cell by the external application of purified recombinant DNA from another cell of different genotype, leading to its uptake and integration into the subject cell's genome. In bacteria, the recombinant DNA is not typically integrated into the bacterial chromosome, but instead replicates autonomously as a plasmid.

Upstream means on the 5′ side of any site in DNA or RNA.

A vector is a nucleic acid molecule that is able to replicate autonomously in a host cell and can accept foreign DNA. A vector carries its own origin of replication, one or more unique recognition sites for restriction endonucleases which can be used for the insertion of foreign DNA, and usually selectable markers such as genes coding for antibiotic resistance, and often recognition sequences (e.g. promoter) for the expression of the inserted DNA. Common vectors include plasmid vectors and phage vectors.

The term “fluorescent tag”, “fluorescent label” or “fluorescent probe”, is a molecule that can be bonded covalently to another molecule such as a small molecule modulator of biological signaling, via direct attachment, a tether, or linker to aid in the detection of a biological process. Fluorescein and green fluorescent protein are examples of tags.

### Embodiments of the Invention

This disclosure provides a compound of Formula I:

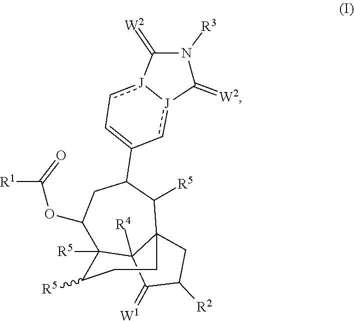

or stereoisomer or salt thereof; wherein

J is CH or N and [symbol] is a single bond, or J is C and [symbol] is a double bond;

R¹ is halo, OH, —(C₁-C₆)alkyl-X, —(C₂-C₆)alkenyl-X, or heteroaryl, wherein the (C₁-C₆)alkyl moiety of —(C₁-C₆)alkyl-X is substituted optionally with one or more (e.g., 1, 2, 3, etc.) halo groups;

X is absent, halo, OH, or —O(C═O)CH₃;

R² and R⁴ are independently H, halo, or OR^{A} wherein R^{A} is H, —(C₁-C₆)alkyl, or —(C═O)CH₃;

R³ is aryl, heteroaryl, heterocycloalkyl, —(C₃-C₆)cycloalkyl, —(C₂-C₆)alkynyl, or a group comprising a fluorescent tag, wherein aryl or heteroaryl is substituted optionally with halo, OH or —(C₁-C₆)alkyl;

each R⁵ is independently H or —(C₁-C₆)alkyl;

W¹ is absent, O or S; and each W² is independently absent, O or S.

When X is absent (i.e., H of the parent alkyl), R¹ is halo, OH, —(C₁-C₆)alkyl, —(C₂-C₆)alkenyl, or heteroaryl, wherein —(C₁-C₆)alkyl is optionally substituted with halo. When W¹ or W² is absent then the carbon atom directly attached to W¹ or W² is CH₂. In some embodiments, the (C₂-C₆)alkenyl moiety of —(C₂-C₆)alkyl-X is substituted optionally with halo.

In embodiments, J is N and [symbol] is a single bond. In other embodiments, J is CH and [symbol] is a single bond. In some other embodiments, R¹ is —(C₁-C₆)alkyl-X. In yet other embodiments, R¹ is —CH₃, —CH₂F, —CH₂Cl, —CH₂I, —CH₂O(C═O)CH₃, —CHCl₂, vinyl, allyl, ethynyl, propynyl, or 2-furanyl. In additional embodiments, R² and R⁴ are OR^{A}. In further embodiments, R³ is aryl or —(C₂-C₆)alkynyl. In some other embodiments, R³ is phenyl or propynyl. In yet other embodiments, W¹ and W² are O. In some embodiments, R⁴ is OH and R⁵ is CH₃. In additional embodiments, R³ is phenyl, J is N, and [symbol] is a single bond.

Also, this disclosure provides a compound of Formula I that is a compound of Formula II, III, or IV:

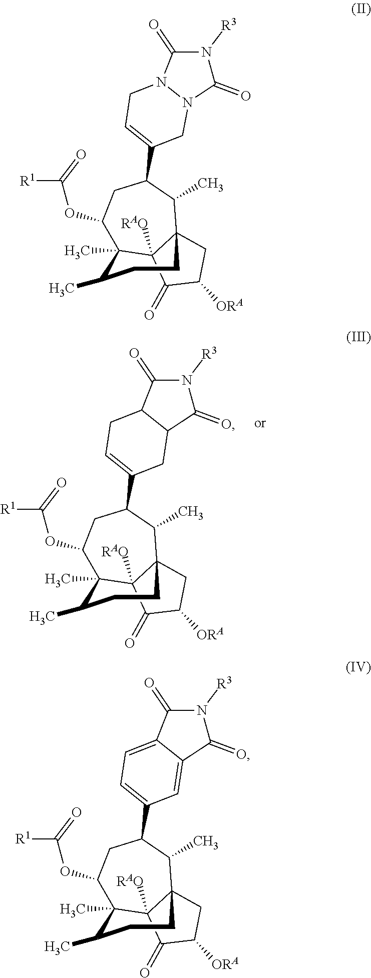

wherein

R¹ is —CH₃, —CH₂F, —CH₂Cl, —CH₂I, —CH₂O(C═O)CH₃, —CHCl₂, vinyl, allyl, ethynyl, propynyl, or 2-furanyl; and each R^{A} is independently H, —(C₁-C₆)alkyl, or —(C═O)CH₃.

In some embodiments, R³ is phenyl, propynyl, or a group comprising a fluorescent tag. In other embodiments, the compound of Formula I is:

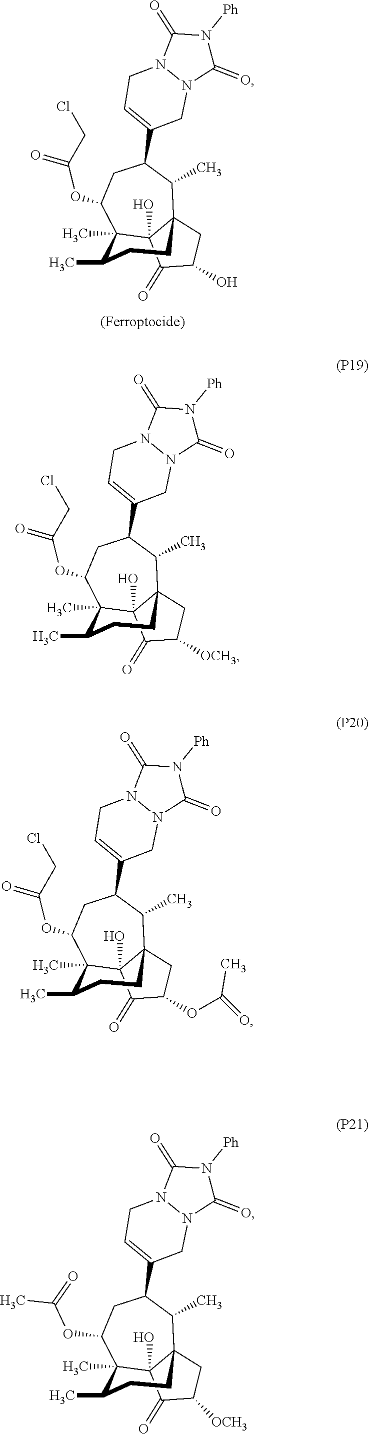

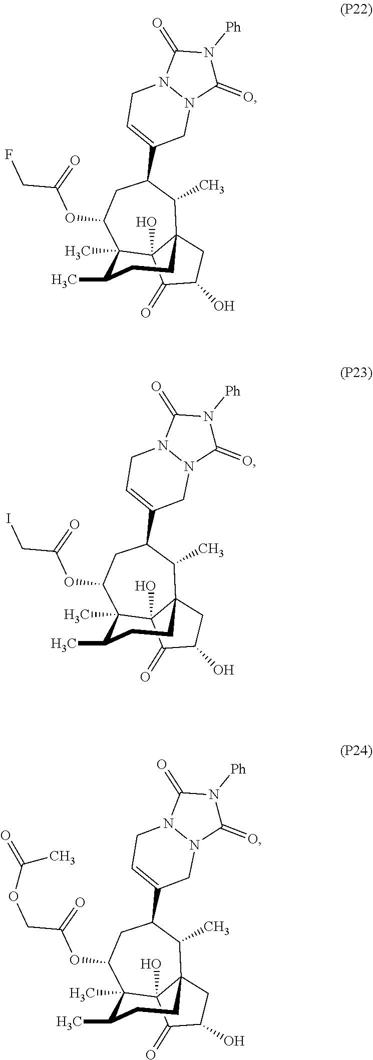

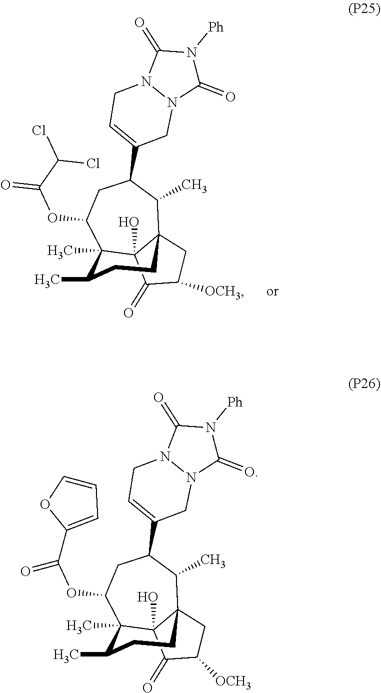

In yet other embodiments, the compound of Formula I is:

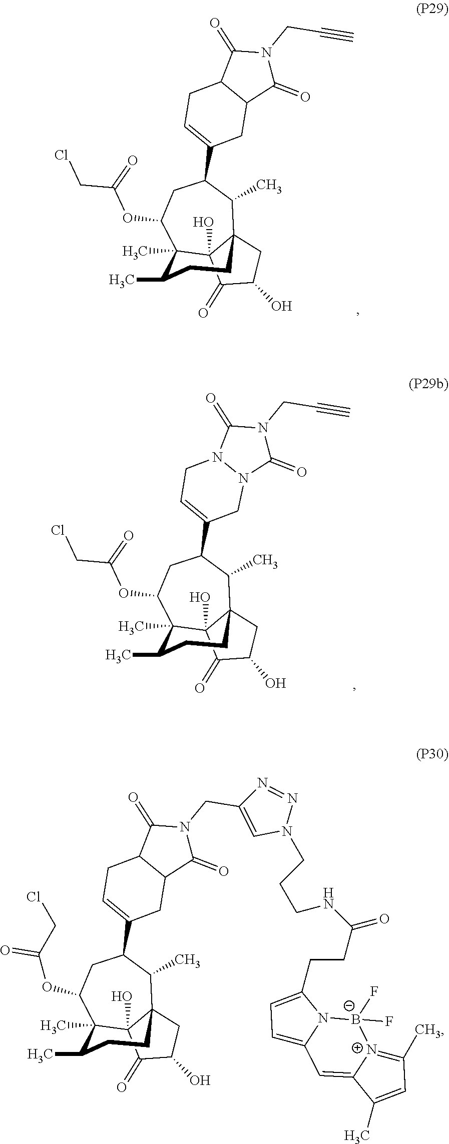

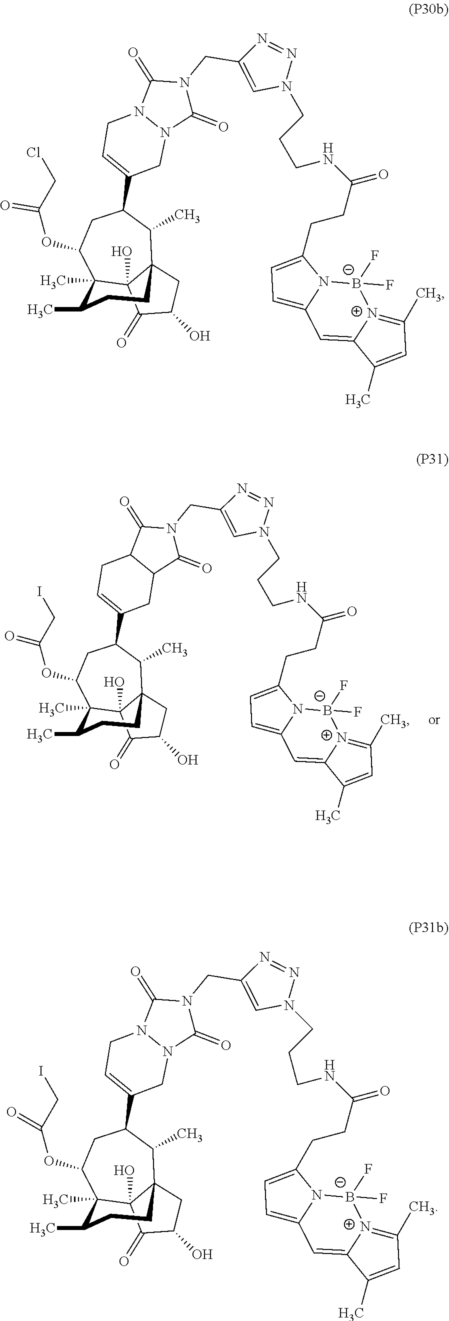

In various embodiments, the compound is an inhibitor of thioredoxin and thioredoxin is covalently modified by the compound.

Additionally, this disclosure provides a composition comprising a compound disclosed above and a pharmaceutically acceptable buffer, carrier, diluent, or excipient.

This disclosure also provides a method for inducing ferroptosis in cancer cells comprising contacting the cancer cell with an effective amount of a compound disclosed above, thereby inducing ferroptosis. In various embodiments, the IC₅₀ of the compound inducing ferroptosis in cancer cells is about 1 nanomolar to about 50 micromolar. In other embodiments, the IC₅₀ of the compound is about 1 nanomolar to about 0.1 micromolar, about 0.1 micromolar to about 1 micromolar, about 1 micromolar to about 5 micromolar, about 5 micromolar to about 10 micromolar, about 10 micromolar to about 20 micromolar, about 20 micromolar to about 30 micromolar, about 30 micromolar to about 40 micromolar, about 40 micromolar to about 50 micromolar, about 50 micromolar to about 75 micromolar, or about 75 micromolar to about 100 micromolar.

In various other embodiments, the compound includes a group comprising a fluorescent tag and the cancer cell thereby is fluorescently labeled.

Additionally, this disclosure provides a method for treating cancer in a cancer subject comprising administering an effective amount of a compound disclosed above to the cancer subject in need of cancer treatment wherein the cancer is thereby treated. In additional embodiments, the cancer is blood cancer, brain cancer, breast cancer, colorectal cancer, liver cancer, lung cancer, ovarian cancer, pancreatic cancer, prostate cancer, or skin cancer.

Also, this disclosure provides a method for modulating the immune system in a subject comprising administering an effective amount of a compound disclosed above to the subject in need of immunostimulation wherein the immune system of the subject is thereby modulated. In various embodiments, the compound is Ferroptocide.

### Results and Discussion

Diversifying Pleuromutilin. The diterpene natural product P is found in several species of fungi and is a potent inhibitor of the bacterial 50S ribosome. P is composed of 5-, 6-, and 8-membered rings and contains eight contiguous stereogenic centers. Several semisynthetic derivatives of P are used to treat Gram-positive pathogens in humans (retapamulin) and in veterinary medicine (tiamulin, valnemulin), and recently epi-mutilin derivatives have been developed as antibiotics with activity against some Gram-negative bacteria. Investigation of the antibacterial activity of P and its derivatives has inspired several total synthesis efforts that, combined with previous work on structure elucidation and structure-activity relationship studies, provide a wealth of synthetic information about the chemical reactivity of the pleuromutilin ring system. Structural transformations of the P ring system identified through these efforts afford several good starting points for novel diversification reactions. With the objective of harnessing the advantages of P as a starting material to construct a small set of highly complex and structurally diverse compounds, we set out to alter the ring systems of pleuromutilin through a series of ring contraction, expansion, cleavage, and fusion reactions (see Scheme A above).

Treatment of pleuromutilin with phosphorus pentachloride results in activation of the secondary alcohol, carbocation rearrangement, and ring contraction to form known diene P1 (Scheme Aa) as a single diastereomer. Compound P1 is an outstanding starting point for the construction of novel compounds with unusual oxidation patterns and ring systems. For example, silylation of P1 results in formation of the P2 kinetic silyl enol ether. Subsequent Rubottom oxidation induces an alcohol-directed epoxidation on the less hindered face of the five-membered ring, yielding epoxide P3 as a single diastereomer (Scheme Aa). Desilylation of P3 to P4 (Scheme Aa) provides an α-hydroxy ketone for further manipulation, and exposure of the α-hydroxy ketone of P4 to lead tetraacetate produces a novel rearrangement yielding P5. This proceeds through oxidative cleavage of the less hindered C—C bond of the hydroxy ketone, resulting in an intermediate containing an aldehyde and ester. Hemiacetal formation occurs between the tertiary alcohol and aldehyde, followed by subsequent lactonization, thus efficiently installing two new stereocenters via diastereoselective oxidation and resulting in a ring rearrangement to form P5.

In addition to the direct formation of P1 from pleuromutilin, the use of carbocation rearrangements is a useful strategy for inducing dramatic changes to the overlapping rings found in pleuromutilin. To construct a subset of compounds in which the 6-membered ring was expanded, we identified alcohol P6, a common intermediate in the synthesis of pleuromutilin-derived antibiotics, as a useful substrate (Scheme Ab). P6 was generated via acid catalyzed isomerization and subsequent 1,5-hydride shift of pleuromutilin. Treatment of P6 with PCl₅ resulted in a novel expansion of the 6-membered ring and elimination to form a new scaffold, P7, as a single isomer. Further modification of P7 was accomplished by diastereoselective epoxidation of the disubstituted olefin to afford novel scaffold P8. Elimination and epoxide opening of P8 forms new allylic alcohol P9 (Scheme Ab).

Cleavage of the 8-membered ring of P has been reported to occur via a retro-Michael reaction. Indeed, oxidation of the secondary alcohol of pleuromutilin affords a 1,5-diketone that after retro-Michael ring cleavage with potassium hydroxide, and oxidation of the resulting ketal with pyridinium chlorochromate provides known lactone P10 (Scheme Ac). Compound P10 was then used to construct novel lactam P12, through ring expansion of the cyclopentanone ring of P10 induced by oxime formation (P11) and Beckmann rearrangement initiated by cyanuric chloride (Scheme Ac).

Finally, ring fusion to the 8-membered ring of P was achieved by intramolecular C—H insertion of a primary carbamate (Scheme Ad). Inspired by previous work on C—H amidation of the epi-mutilin scaffold, ring fusion precursor P13 was synthesized by saponification of pleuromutilin followed by acylation and carbamylation. Intramolecular C—H nitrene insertion was accomplished using modified silver-catalyzed methodology to provide the novel ring system found in carbamate P14. Exposure of this product to alkaline autoxidation conditions results in a formal ring expansion of the 5-membered ring via enolate formation, oxidative cleavage, hydride transfer, and lactonization to provide novel lactone P15 (Scheme Ad).

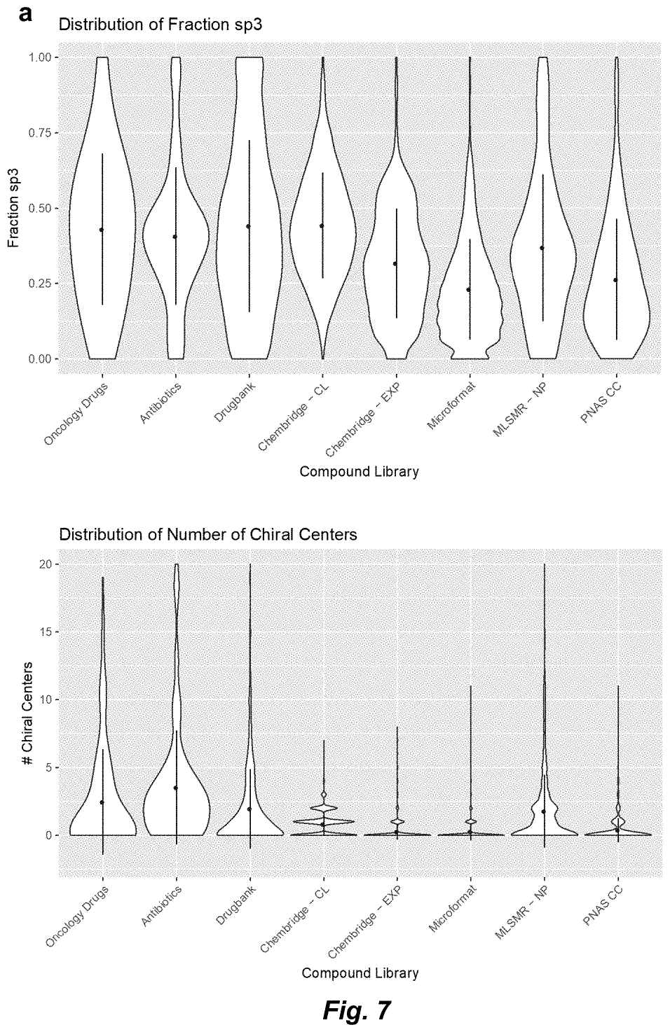

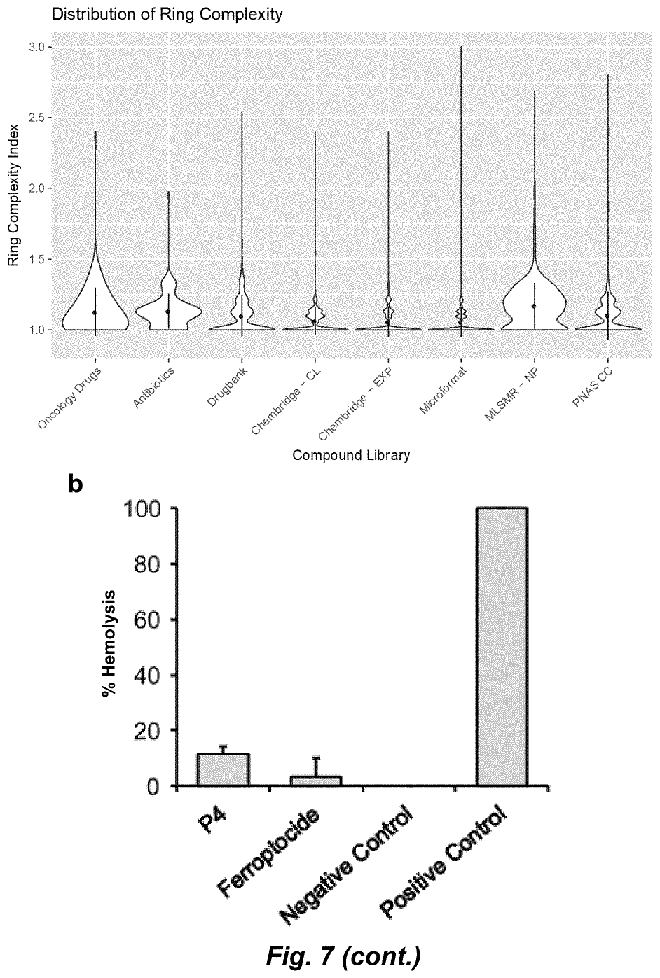

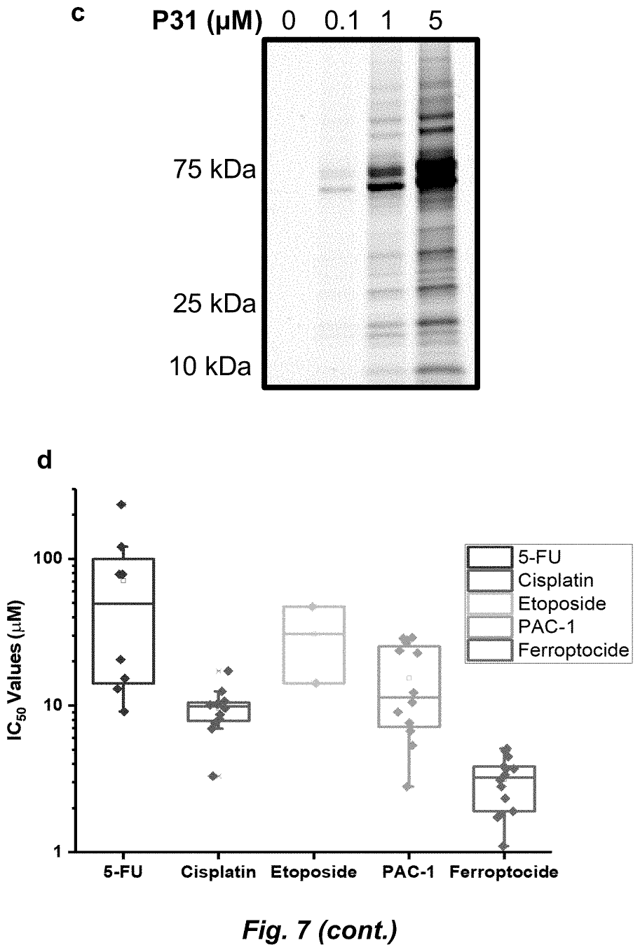

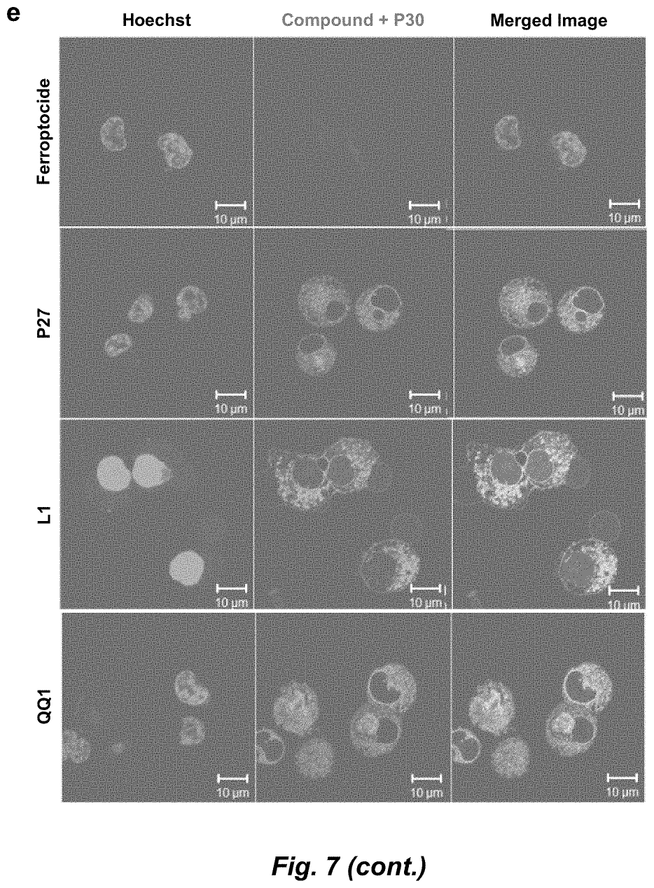

Through the efforts reported herein, 12 structurally complex compounds with novel ring systems were constructed from P, in addition to 6 compounds that had been previously reported; the majority of these were synthesized on ≥25 mg scale. In addition, the compound collection derived from P that was screened (vide infra) also included 11 compounds synthesized in our previously reported transformation of pleuromutilin to P16 and carbocation rearrangement to afford bridged oxafenestranes such as P17 (Scheme Ae). The fraction of sp³-hybridized carbons (Fsp3), the number of stereogenic centers, and ring complexity index were used as surrogates of complexity for the compounds synthesized from pleuromutilin, and these values compare favorably to compounds in screening collections as shown in the violin plots in FIG. 7a.

Anticancer phenotypic screening and compound optimization. Compounds from P were evaluated in whole-cell assays for their ability to rapidly kill cancer cell lines in culture, starting with the ES-2 (ovarian cancer) cell line. All compounds were assessed at 12 μM, with cell viability determined using the Alamar Blue viability assay. Compounds that elicited at least 50% cell death were considered hit compounds and were then evaluated through full dose-response curves. From these assessments, compound P4 was identified as having promising activity, with rapid induction of cell death, an IC₅₀=6.7 μM in ES-2 cells (Scheme B), and counter-screening revealed that this small molecule displayed no signs of hemolytic activity (FIG. 7b).

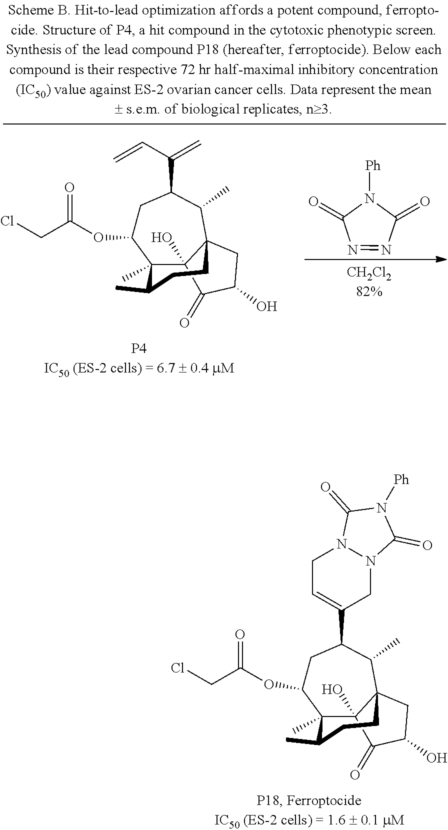

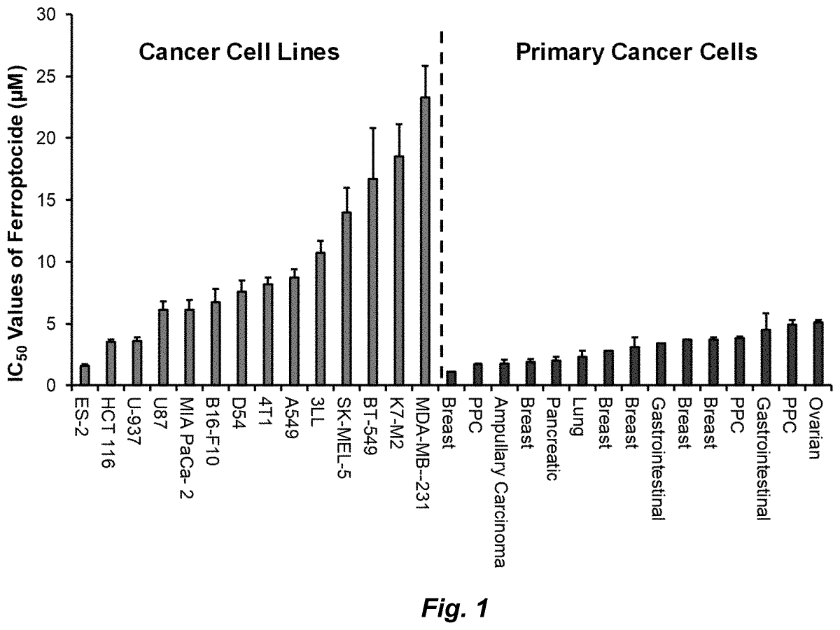

Modification of P4 through a [4+2] cycloaddition provided compound P18, hereafter referred to as ferroptocide (Scheme B); this compound is more potent than P4, with an IC₅₀=1.6 μM against ES-2 cells. Further modification of the secondary alcohol and the α-chloro ester of ferroptocide unveiled structural features important for activity. While methylation and acetylation of the secondary alcohol of ferroptocide (P19 and P20 respectively) did not change the activity (Table 1), replacement of the α-chloro ester with acetate (P21) eliminated activity. To investigate other electrophilic groups the fluoro- (P22) and iodo- (P23) compounds were synthesized. While P22 had greatly diminished anticancer activity, iodo analogues (such as P23) showed greater potency in cells at the expense of biological selectivity (Scheme Ac), and because of this promiscuity such compounds were not pursued further. Additional compounds with poorer leaving groups such as α-acetate ester (P24), α,α dichloro ester (P25), and furoic ester (P26) exhibited no anticancer activity (Table 1). Lead compound ferroptocide displays no antibacterial activity (MIC>64 μg/mL) in gram positive (S. aureus) or gram-negative (E. coli) bacteria but has robust anticancer activity in a panel of cancer cell lines and, notably, primary cancer cells freshly isolated from tumor tissues of 15 different patients with diverse metastatic cancers (FIG. 1). Ferroptocide kills these cancer cells better than approved and experimental chemotherapeutics such as cisplatin, 5-FU, etoposide, and PAC-1 (FIG. 7d).

TABLE 1
Structure-activity relationship studies of P18 analogues, bioactivity
is expressed as a 72 hr IC₅₀ value against ES-2 cell line as measured
by Alamar Blue fluorescence. Data represent the mean ± s.e.m.,
n ≥ 3.

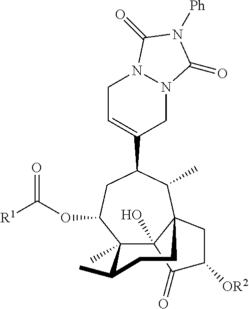

| | R¹ | R² | IC₅₀ (μM) |
|---|---|---|---|
| P18 | 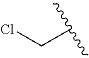 [SMILES: *CCl] | H | 1.6 ± 0.1 |
| P19 | 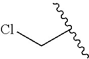 [SMILES: *CCl] | Me | 1.9 ± 0.1 |
| P20 | 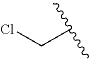 [SMILES: *CCl] | Ac | 1.4 ± 0.5 |
| P21 | 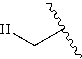 [SMILES: *C] | Me | >100 |
| P22 | 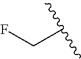 [SMILES: *CF] | H | 34.0 ± 5.7 |
| P23 | 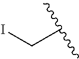 [SMILES: *CI] | H | 0.24 ± 0.002 |
| P24 | 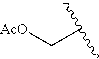 [SMILES: *COC(C)=O] | H | >100 |
| P25 | 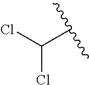 [SMILES: *C(Cl)Cl] | Me | >100 |
| P26 | 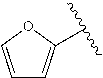 [SMILES: *c1ccco1] | Me | >100 |

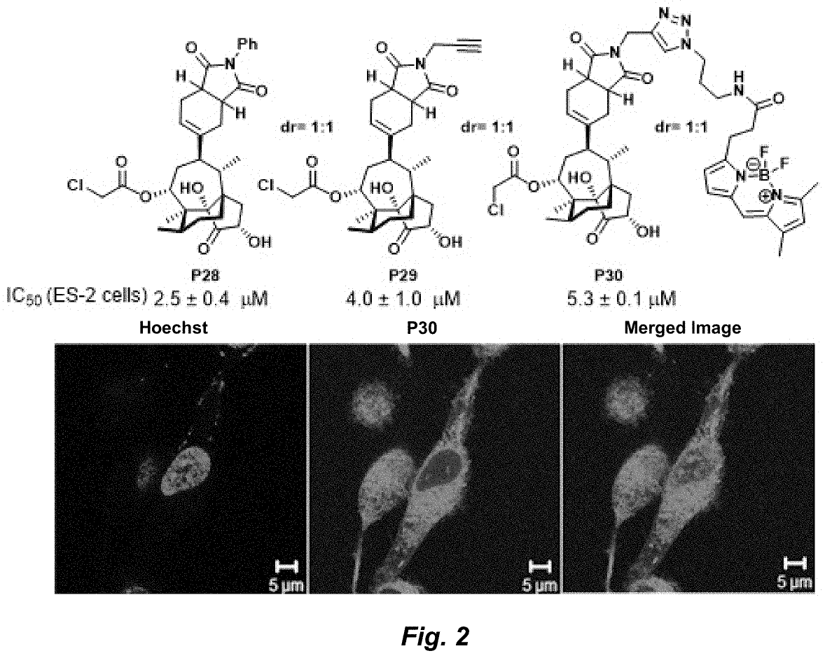

Additional synthesis and evaluation revealed that the N—N moiety in ferroptocide could be changed to C—C (P28), with minimal loss in activity (FIG. 2). This discovery allowed for the construction of alkyne tool compound P29 (FIG. 2), which was subjected to a 1,3-dipolar cycloaddition resulting in fluorescent compound P30. Both P29 and P30 retained anticancer activity similar to ferroptocide (FIG. 2), and P30 was used to report on subcellular localization. As shown by confocal microscopy, P30 localizes to the cytoplasm in ES-2 cells (FIG. 2), and this staining is competed away by pretreatment with ferroptocide (FIG. 7e). Importantly, installation of an α-chloro ester on pleuromutilin itself (P27), and other scaffolds such as lovastatin (L1), and quinine (QQ1), resulted in non-competing compounds in this localization experiment (FIG. 7e), demonstrating that the anticancer activity of ferroptocide is not attributed solely to the presence of the electrophilic functional group.

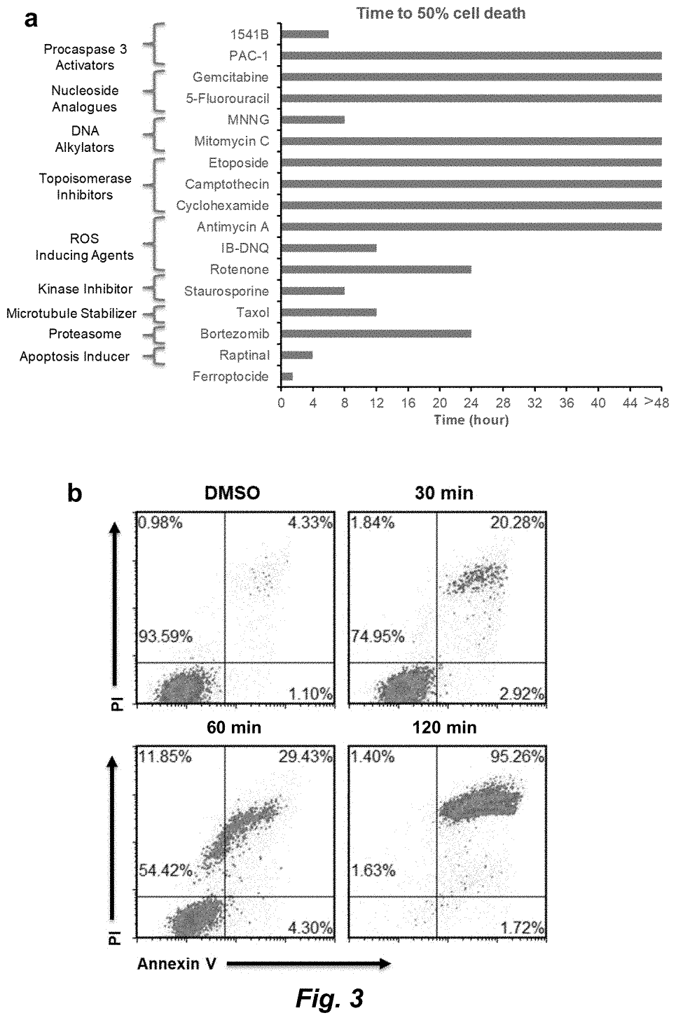

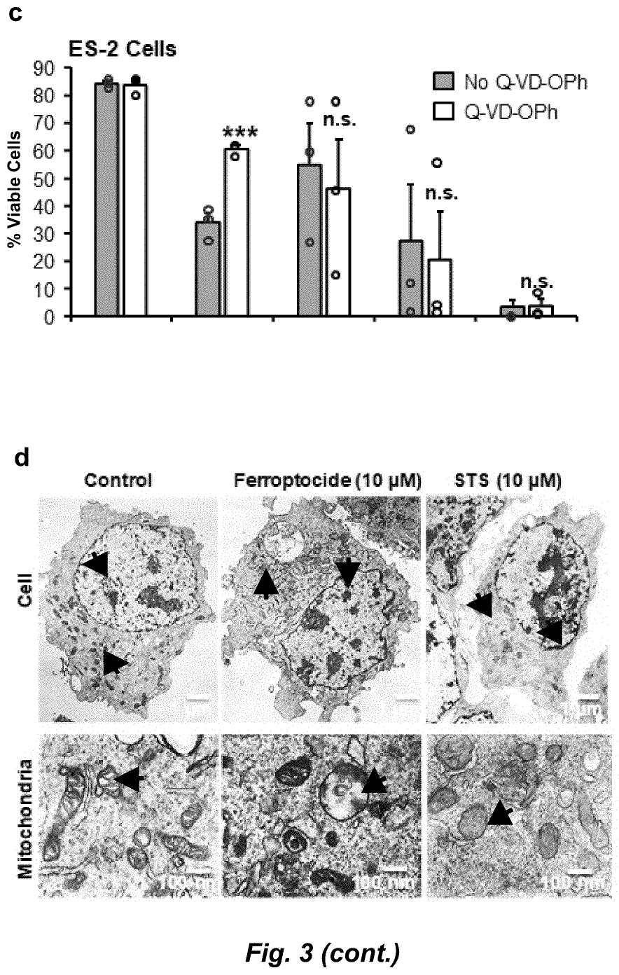

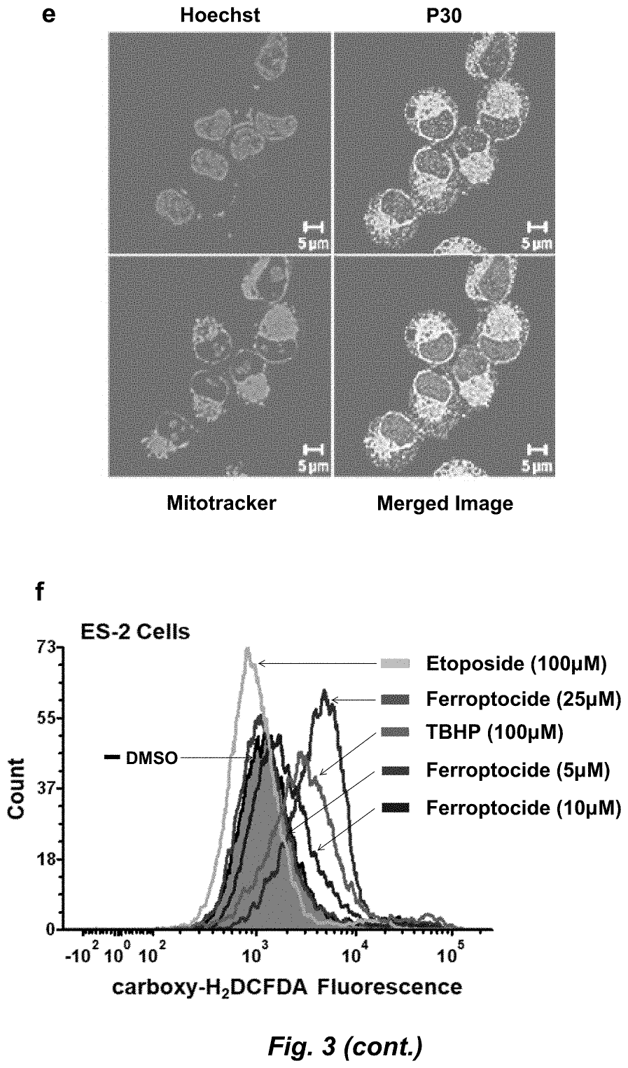

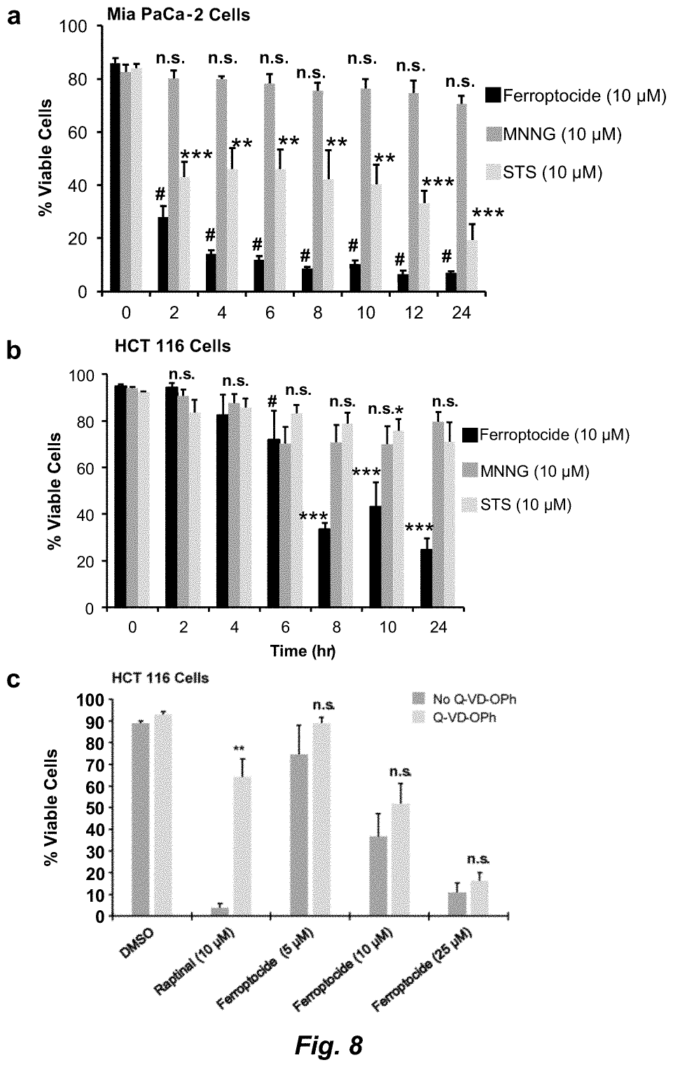

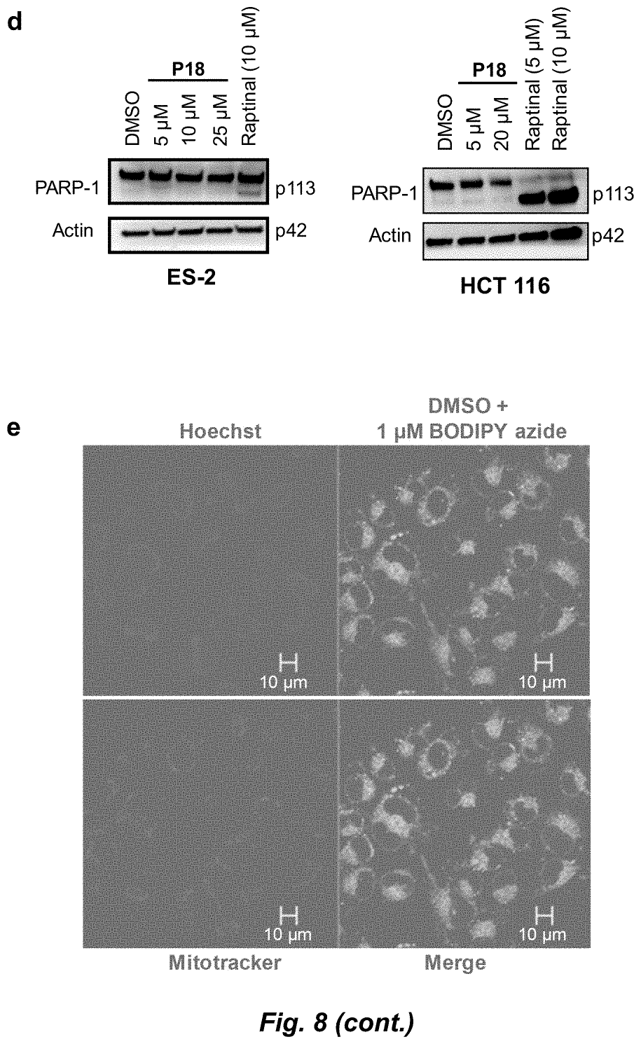

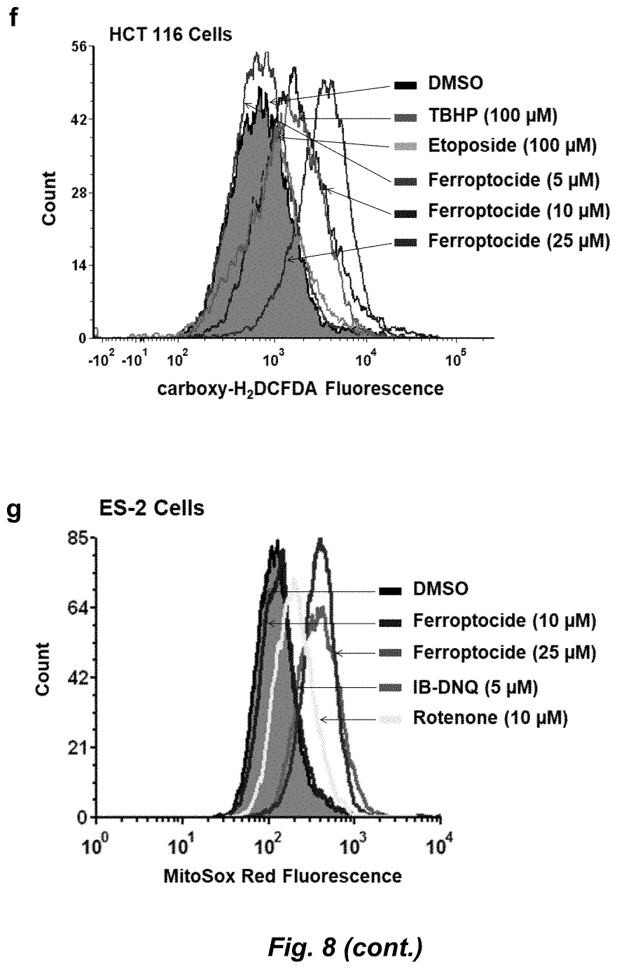

Ferroptocide induces non-apoptotic cell death. To gain insights into the mode of action, the speed of cell death of ferroptocide was compared to other approved chemotherapeutics and tool compounds with well-defined mechanisms including: procaspase-3 activators (PAC-1, 1541B), nucleoside analogues (gemcitabine, 5-FU), DNA alkylators (MNNG, mitomycin C), topoisomerase inhibitors (etoposide, camptothecin, cycloheximide), ROS inducing agents (anitimycin A, IB-DNQ, rotenone), broad-spectrum kinase inhibitor (staurosporine), microtubule stabilizer (taxol), proteasome inhibitor (bortezomib), and a rapid apoptosis-inducing agent (Raptinal). The cell death induced by ferroptocide was rapid in multiple cell lines of diverse cancer types, with a time to 50% cell death of 1 hour in ES-2 (FIG. 3a), 1.5 hr in Mia PaCa-2 (FIG. 8a), and 7 hr in HCT 116 (FIG. 8b) cells. As the speed of cell death induced by ferroptocide was faster than the most rapid proapoptotic agent known (Raptinal), it was suspected to induce non-apoptotic cell death.

Time course analysis of cells treated with ferroptocide followed by Annexin V/PI staining indeed suggested a non-apoptotic mode of cell death (FIG. 3b), as did experiments showing that the pan-caspase inhibitor Q-VD-OPh does not protect against ferroptocide-induced cell death in ES-2 (FIG. 3c) and HCT 116 cells (FIG. 8c). Cleavage of PARP-1 in ferroptocide-treated ES-2 or HCT 116 cells was not observed (FIG. 8d). As a further confirmation, cell morphological changes induced by ferroptocide were examined using transmission electron microscopy (TEM). Cells treated with ferroptocide exhibit none of the apoptotic characteristics such as membrane blebbing and chromatin condensation (FIG. 3d, see staurosporine control). Together, these data indicate that ferroptocide induces rapid, non-apoptotic cell death. As compounds with such a mode of cell death can have unique properties and advantages in vivo, further elucidation of the mechanism of cell death of ferroptocide was of interest.

Further analysis of the TEM images revealed mitochondrial swelling as early as 30 minutes after ferroptocide treatment. Subsequent confocal microscopy studies supported such findings as the fluorescent analogue P30 was found to co-localize with the Mitotracker dye in cells (FIG. 3e) while the BODIPY azide dye alone did not (FIG. 8e). These data suggest a mitochondria-based activity of ferroptocide. Given the importance of reactive oxygen species (ROS) generation in mitochondria, ROS levels were monitored upon compound treatment using a ROS probe, carboxy-H₂DCFDA. Dose-dependent ROS production was observed in ES-2 (FIG. 3f) and HCT 116 cells (FIG. 8f) treated with ferroptocide, similar to the positive control, tert-butyl hydroperoxide (TBHP). Furthermore, treatment of ES-2 cells with ferroptocide results in an increase of mitochondrial ROS similarly to treatment with positive controls IB-DNQ and rotenone (FIG. 8g). Collectively, these data support a disruptive role of ferroptocide on mitochondrial activity.

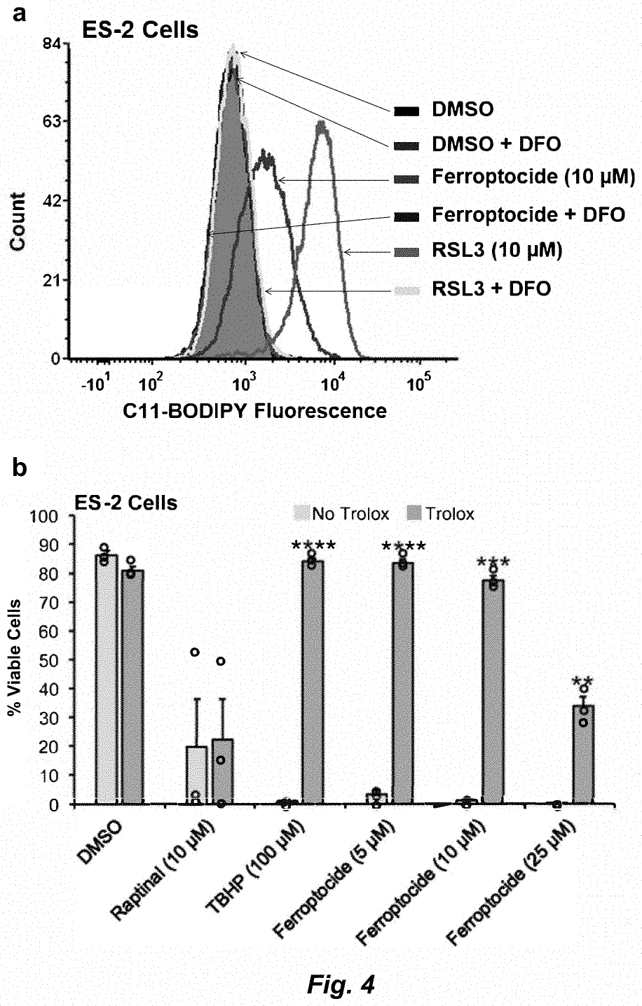

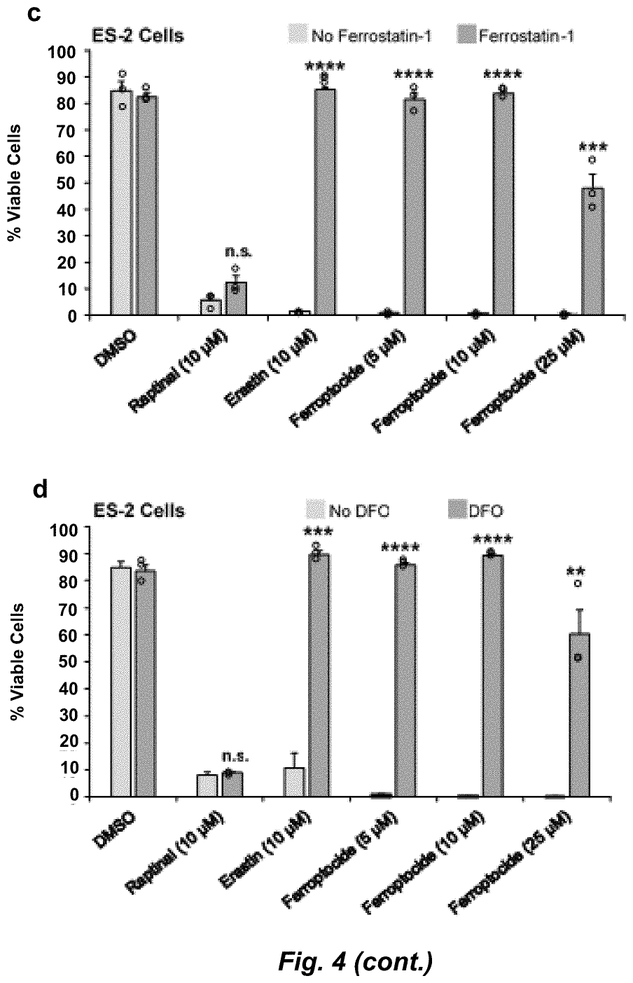

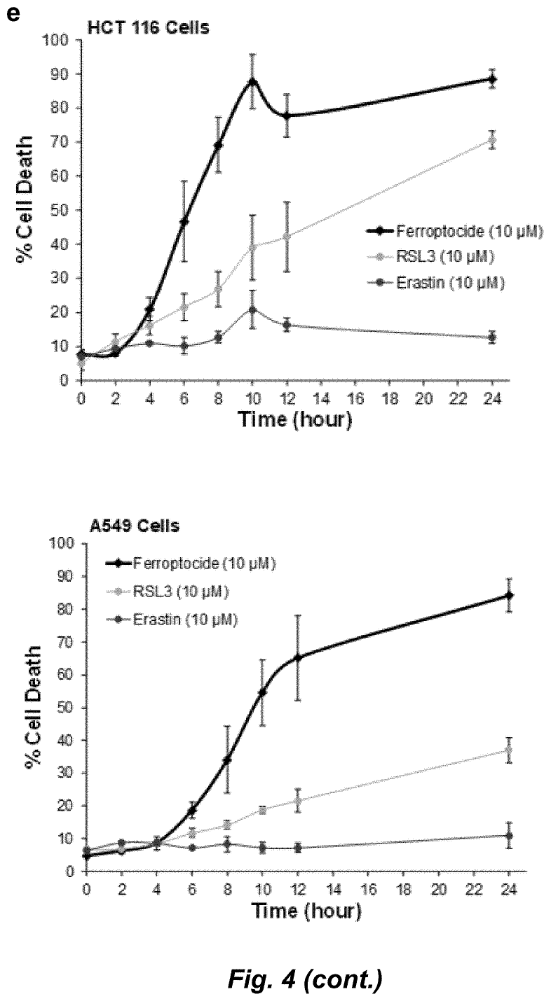

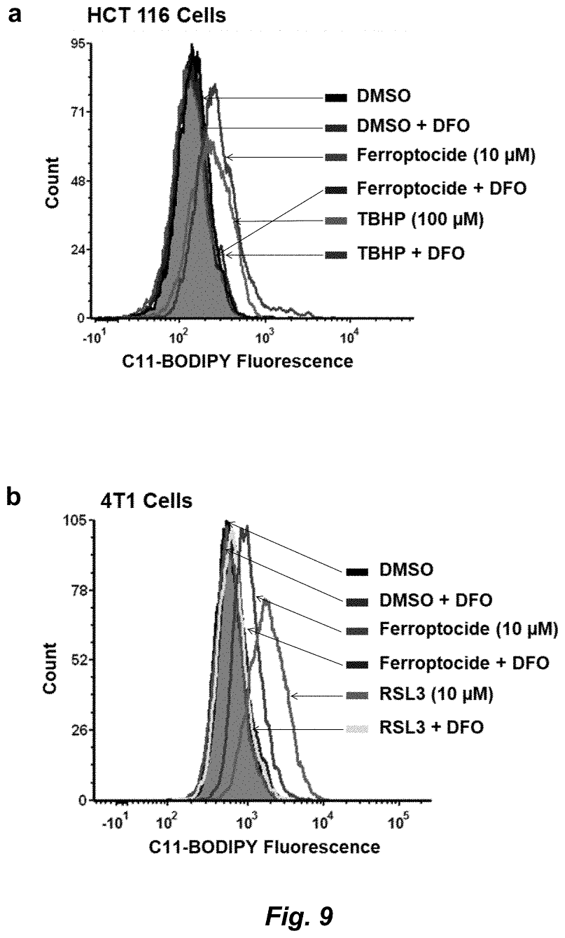

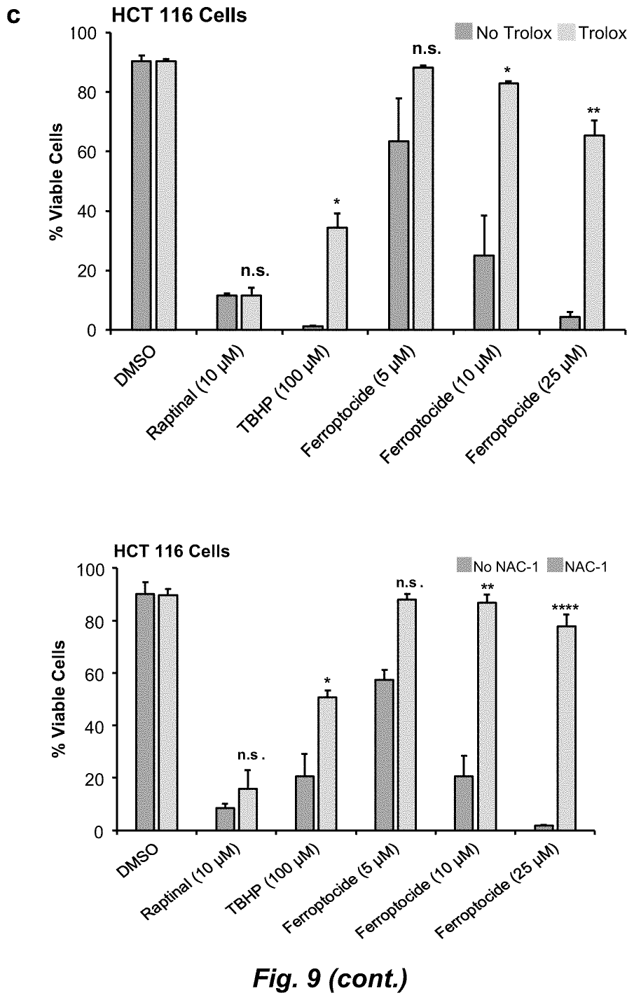

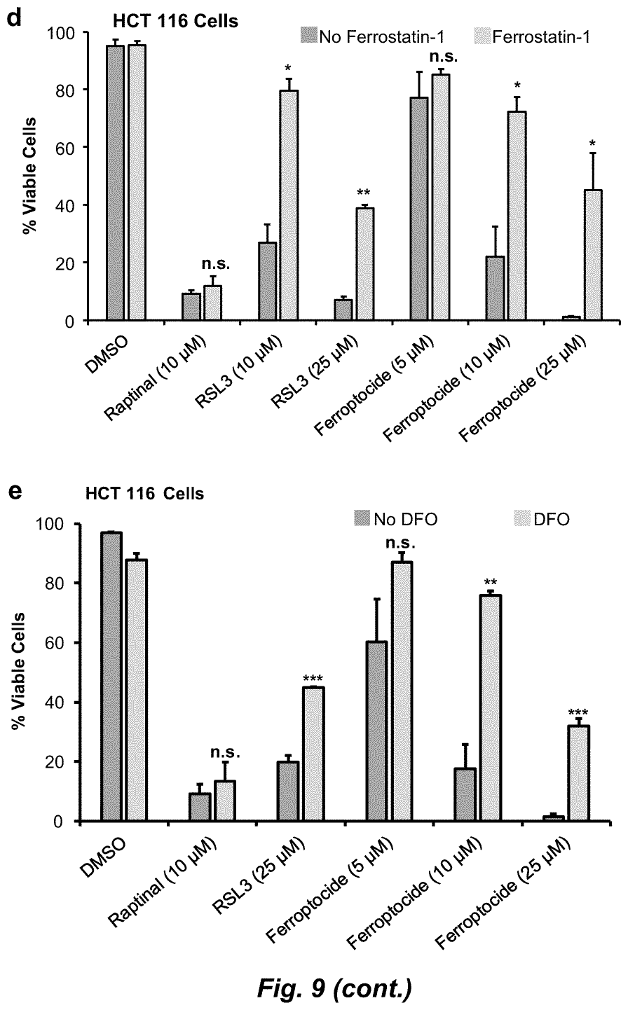

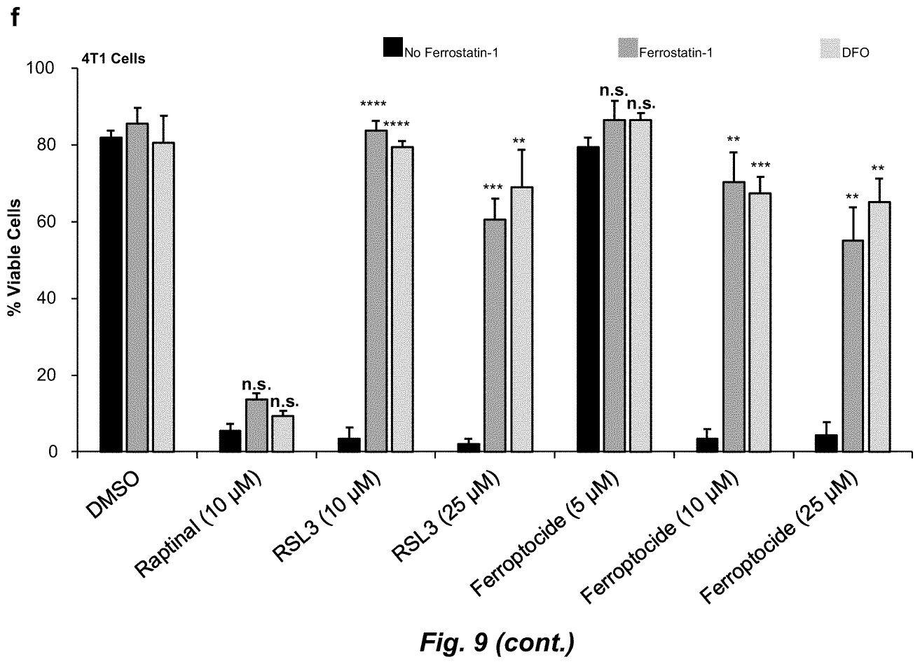

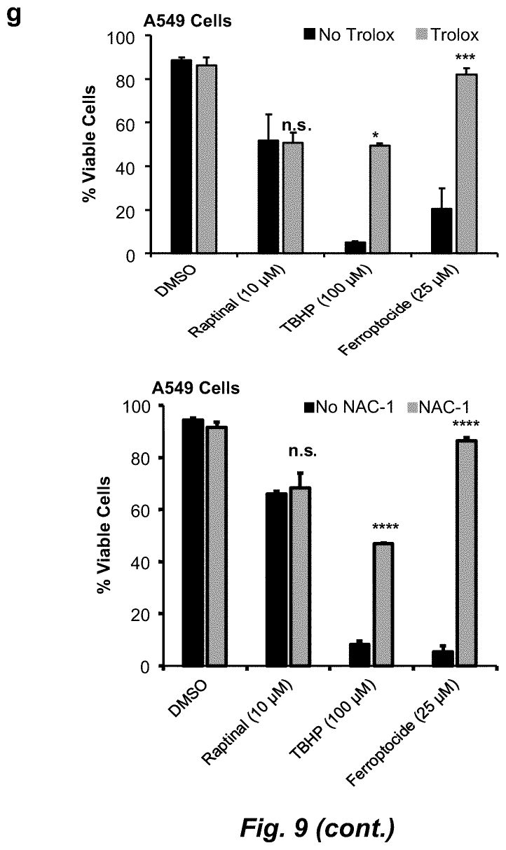

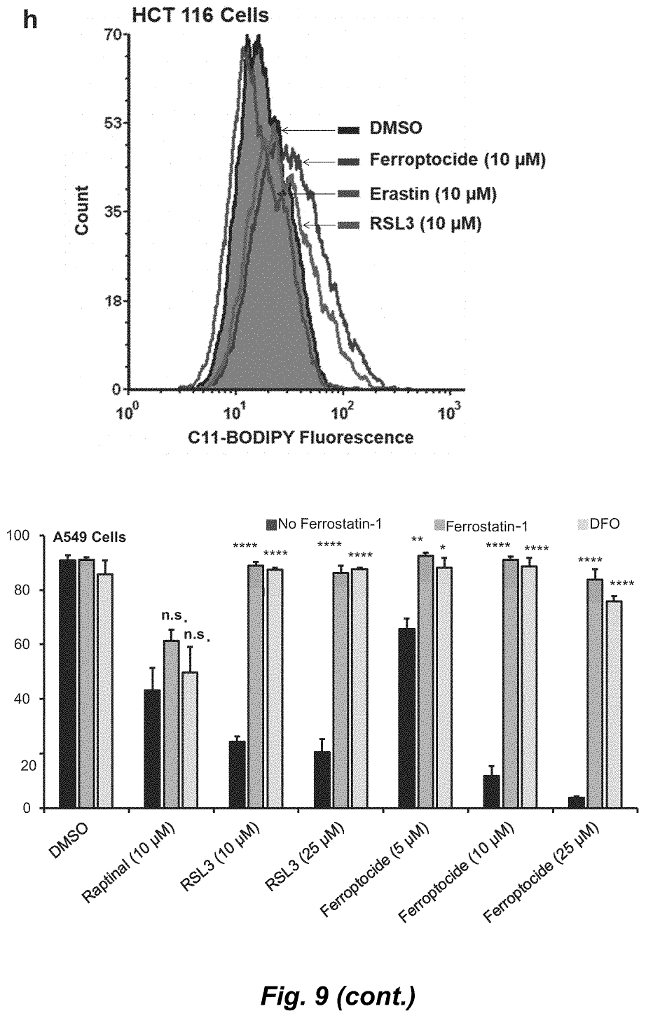

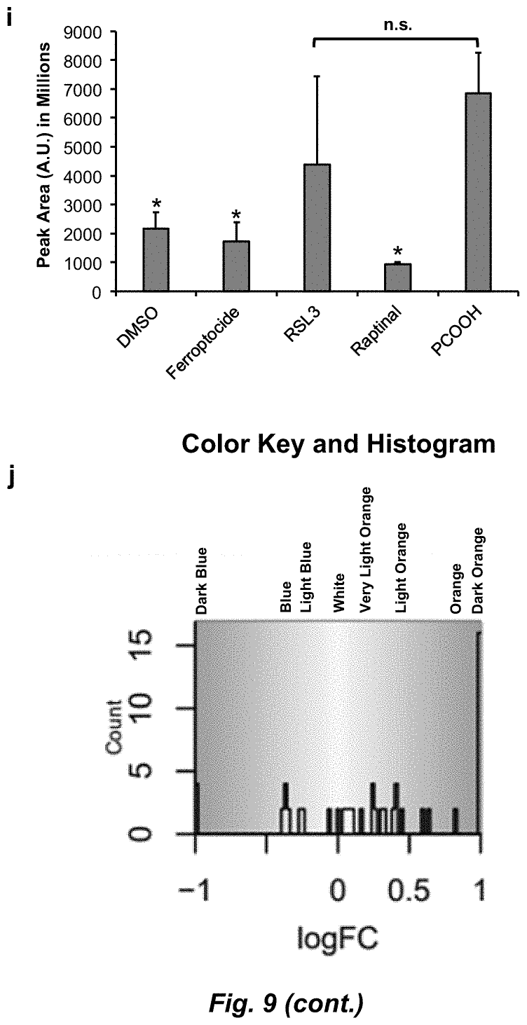

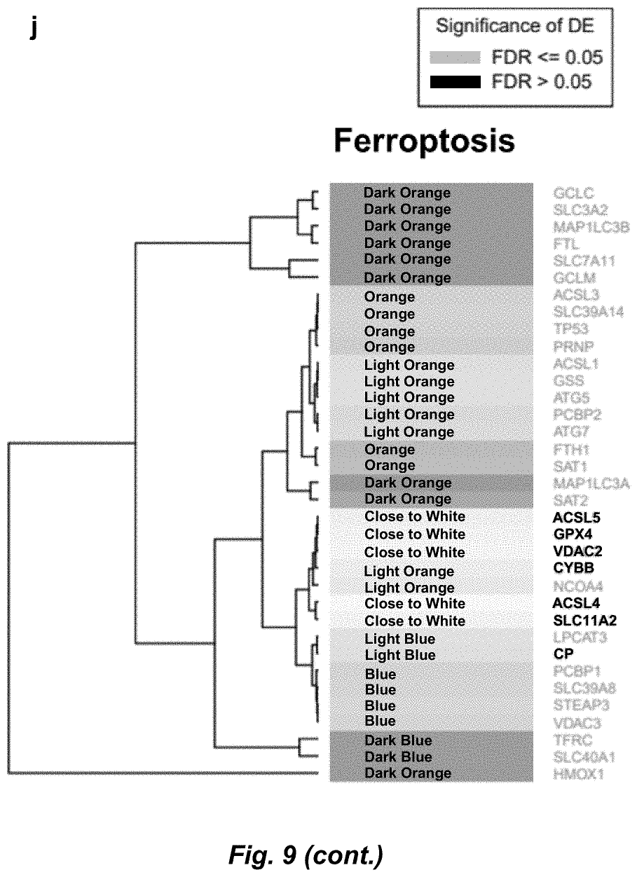

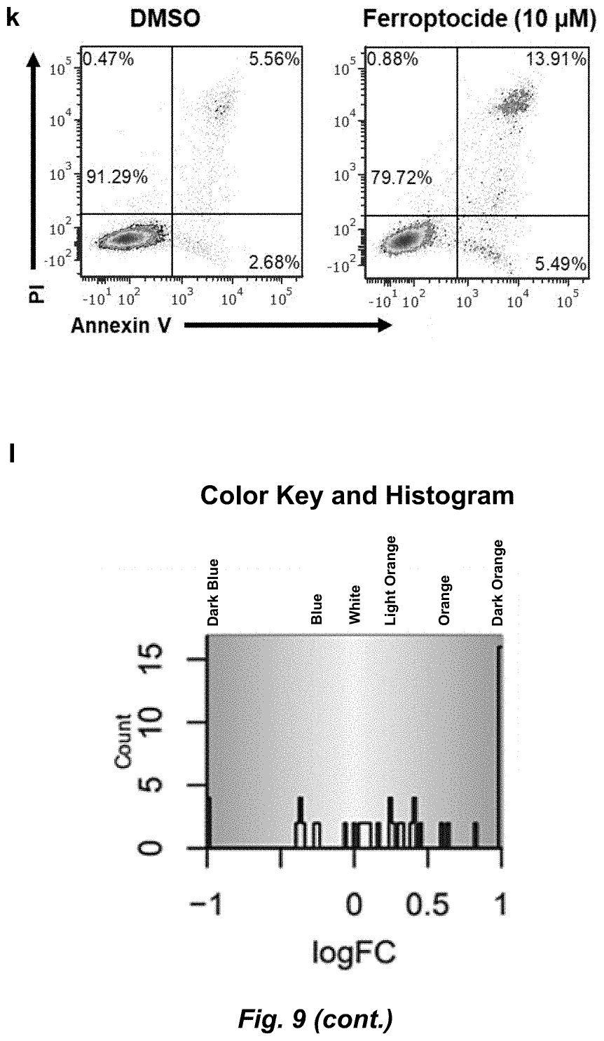

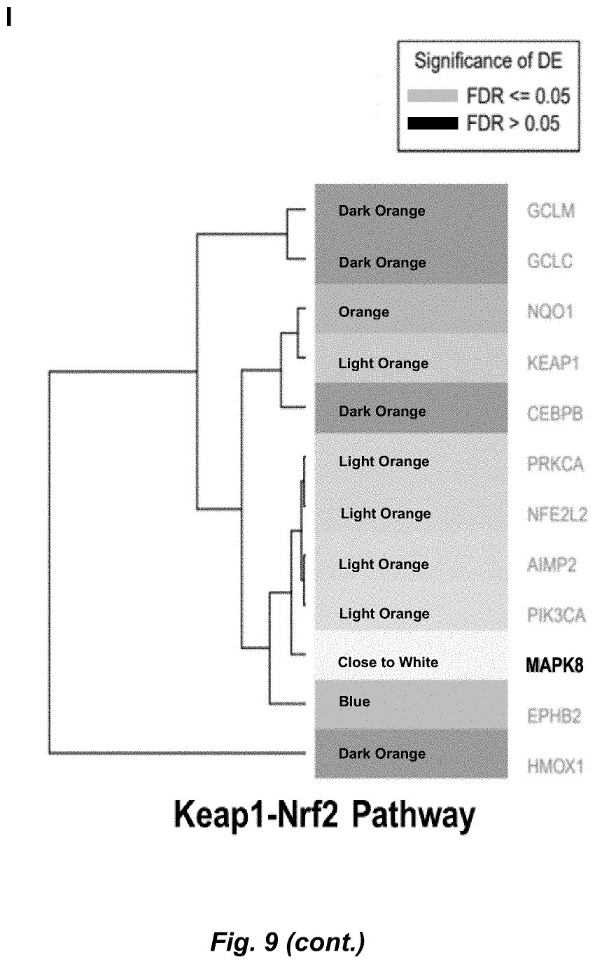

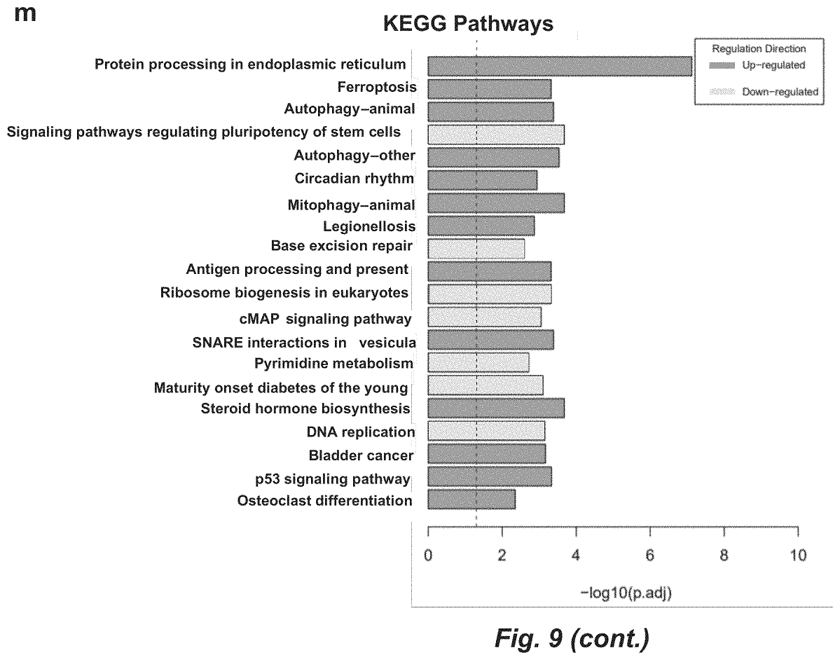

Ferroptocide is a pro-ferroptotic agent. One non-apoptotic mode of cell death that depends on production of lethal levels of iron-dependent lipid ROS is ferroptosis, a regulated process with distinct morphological, biochemical, and genetic characteristics that shares similar features with another non-apoptotic form of cell death, oxytosis. The hallmarks of ferroptosis include generation of lipid hydroperoxides and cytoprotection by lipophilic antioxidants (trolox, butylated hydroxyltoluene [BHT]), ferroptosis inhibitors (ferrostatin-1, liproxstatin), and iron chelators (deferoxamine [DFO], ciclopirox olamine [CPX]). Cellular effects of ferroptocide-induced ROS were investigated using a C11-BODIPY probe that responds to lipid peroxidation. Ferroptocide induces lipid ROS in ES-2 (FIG. 4a), HCT 116, and 4T1 cells (FIG. 9a, b) similar to the known ferroptosis inducer, (1S,3R)-RSL3 (hereafter RSL3) and/or TBHP; DFO pre-treatment of ES-2, HCT 116, and 4T1 cells protected them from lipid ROS induced by ferroptocide, TBHP, and RSL3. Given that generation of continuous lipid ROS is a functional requirement of ferroptosis, additional experiments were conducted to elucidate if ferroptosis was triggered by ferroptocide.

Protection studies were conducted with trolox, ferrostatin-1, and DFO, and all these inhibitors significantly protected against ferroptocide-induced cell death in ES-2 (FIG. 4b, c, d), HCT 116, 4T1, and A549 cancer cells (FIG. 9c-g). Additionally, these inhibitors rescued cells from TBHP-treatment (FIG. 4b and FIG. 9c, g) and the known ferroptosis inducers erastin (FIG. 4c, d) and RSL3 (FIG. 9d-g) respectively, while they showed no protection against Raptinal, an apoptosis-inducing agent. Treatment of HCT 116 and A549 cells with the antioxidant N-acteyl cysteine (NAC-1) resulted in protection from ferroptocide- and TBHP-induced cell death but not Raptinal (FIG. 9c, g) respectively.

Together, these studies indicate that iron-dependent accumulation of lipid peroxidation (ferroptosis) upon ferroptocide-treatment is the cause of cell death.

Erastin and RSL3 were originally discovered as small molecules with RAS-selective lethality. Monitoring of speed of cell death of ferroptocide versus erastin and RSL3 in HCT 116 and A549 cells (which contain mutant oncogenic K-RAS), demonstrates that ferroptocide is a fast-acting, robust pro-ferroptotic agent inducing more quantitative cell death than the other tool compounds (FIG. 4e). Additionally, treatment of HCT 116 cells with the same concentration of these compounds results in generation of similar levels of lipid ROS upon ferroptocide and RSL3 treatment and a larger quantity compared to erastin-treatment, suggesting a rapid onset of lipid peroxidation for ferroptocide and RSL3 (FIG. 9h). Given that RSL3 is a covalent inhibitor of a central regulator of ferroptosis, GPX4, we monitored if ferroptocide modulates the activity of GPX4 in cells. As shown in FIG. 9i, treatment of ES-2 cells with ferroptocide did not result in GPX4 inhibition (in contrast to the positive control RSL3), suggesting a different target for ferroptocide.

Further experiments were conducted to monitor the effect of ferroptocide at the transcript level. RNA-seq data of ferroptocide-treated cells revealed that 35/40 genes involved in ferroptosis are modulated with false discovery rate (FDR) scores ≤0.05 upon 6 hr treatment (FIG. 9j). This time point was selected to capture the primary mechanisms of the compound of interest on viable cells (FIG. 9k). Specific genes such as GCLC (3.5 fold), GCLM (4.9 fold), SLC7A11 (8.1 fold), CHAC1 (9.8 fold) known to be upregulated in ferroptosis and endoplasmic reticulum stress (ATF3 11.5 fold, DDIT3 22.5 fold, DDIT4 11.3 fold), were significantly upregulated after ferroptocide-treatment, similar to RNA-seq reports for erastin in HT-1080 cells. Pathways affected by oxidative stress such as Keap1-Nrf2 (p=7.8 10⁻¹⁰), unfolded protein response (p=3.6 10⁻⁸), protein processing in endoplasmic reticulum (p=2.4 10⁻¹⁰), and others were also modified upon compound treatment (FIG. 9l, m). These transcription profiles provide further support that ferroptocide induces oxidative stress and ferroptosis.

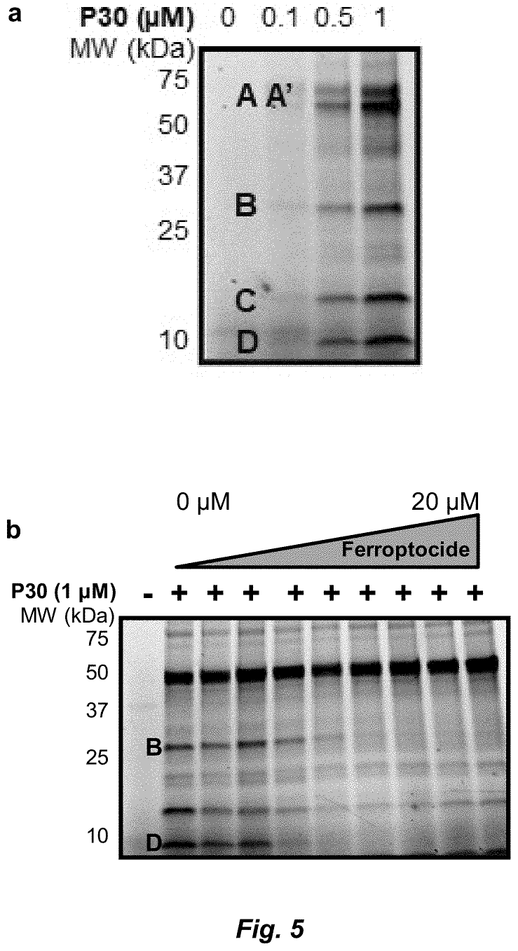

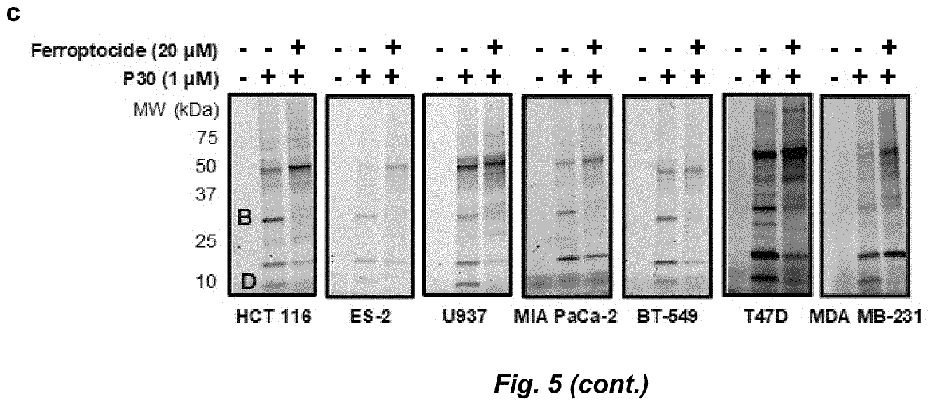

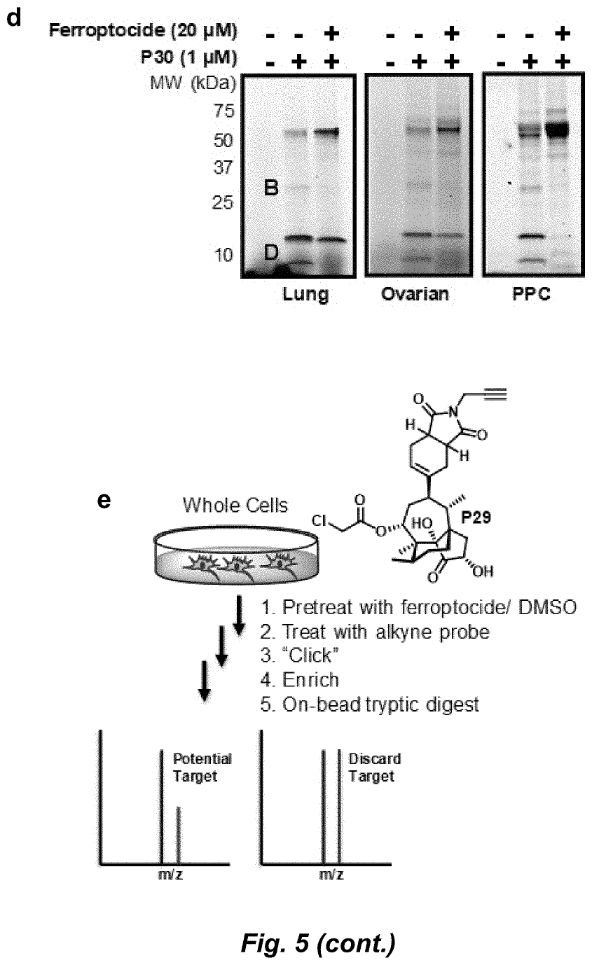

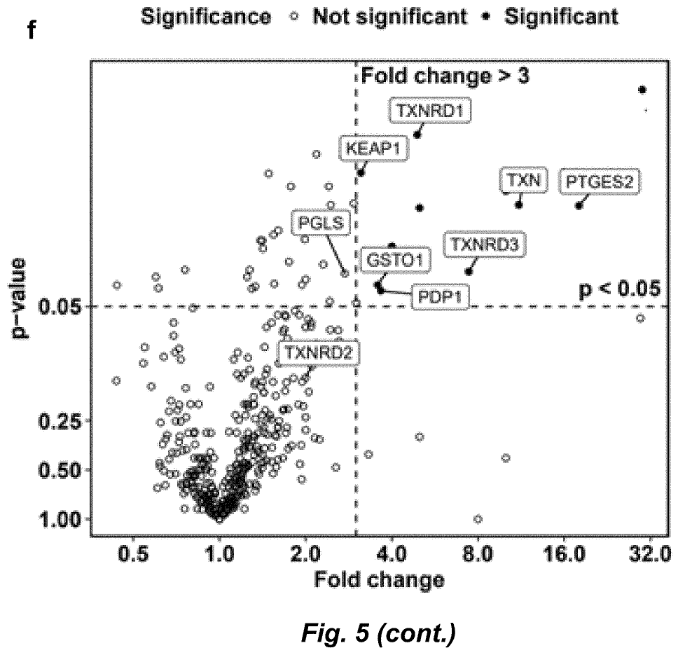

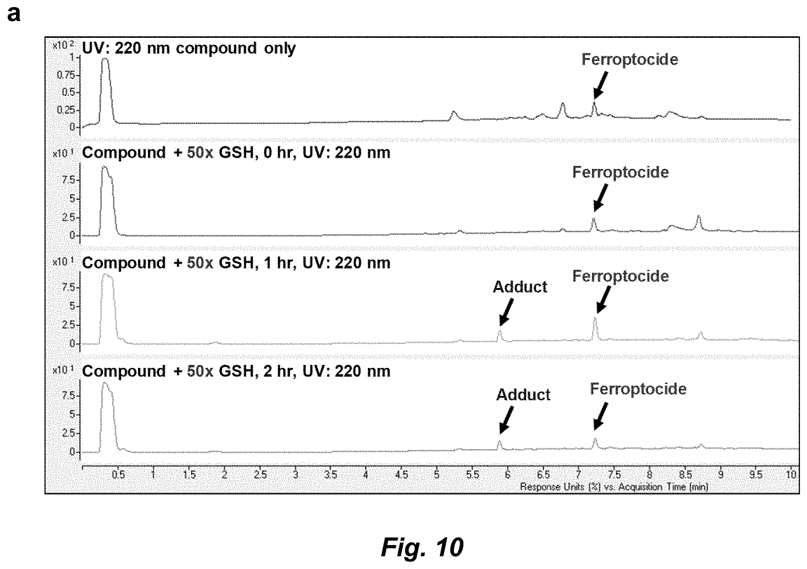

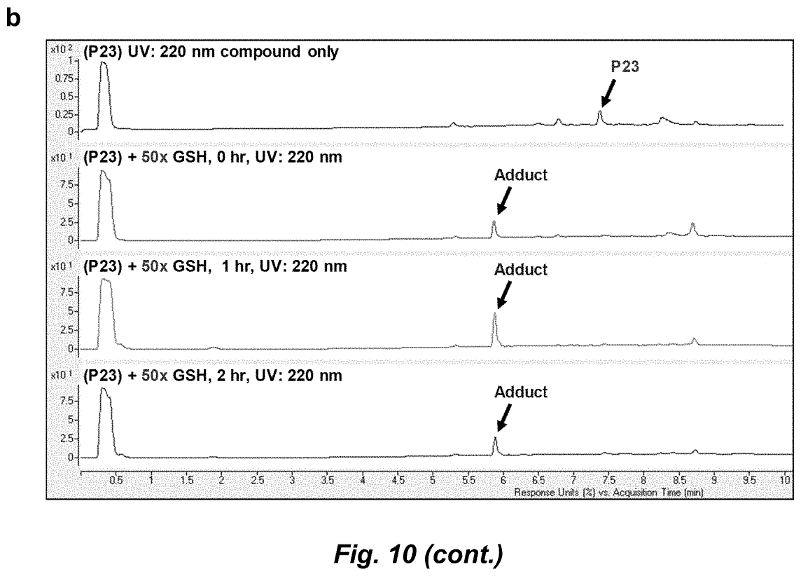

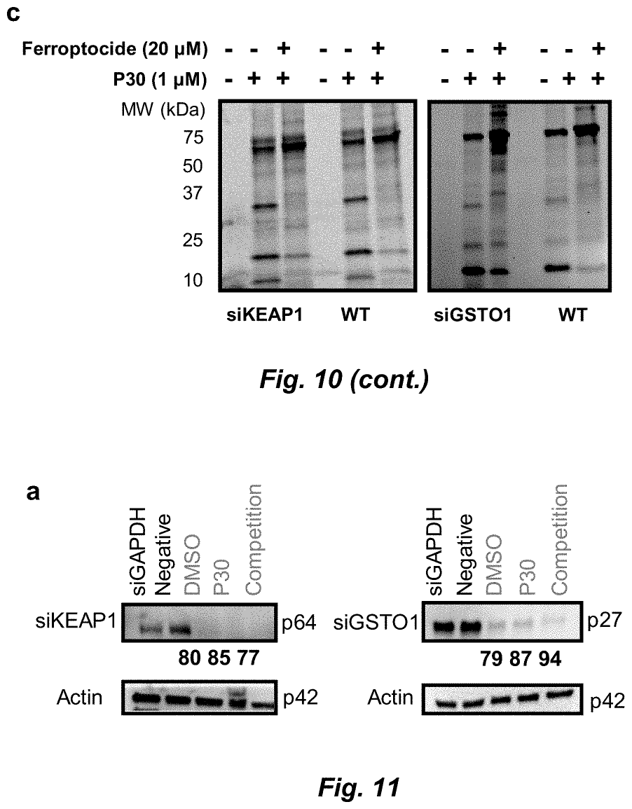

Ferroptocide covalently modifies thioredoxin. SAR trends reveal that ferroptocide-bioactivity depends on the presence of the electrophilic α-chloroester (Table 1), suggesting covalent modification of its target. In vitro studies indicate that ferroptocide reacts slowly with excess glutathione (67% compound remaining after 2 hr) compared to the rapid reaction of the promiscuous iodo analogue, P23 (FIG. 10a-b and Scheme C). To assess covalent modification in cells, in-gel fluorescence studies were performed in conjunction with competition studies. Treatment of HCT 116 cells with increasing concentrations of fluorescent analogue P30 resulted in labeling of five main bands (FIG. 5a). Pretreatment of cells with various concentrations of ferroptocide, followed by treatment with P30 resulted in dose-dependent competition, primarily of two bands, in the in-gel fluorescence assay (bands B and D in FIG. 5b). A similar labeling and competition pattern was observed in multiple cancer cell lines, including HCT 116, ES-2, U937, MIA PaCa-2, BT-549, T47D, MDA-MB-231 (FIG. 5c), and primary cancer cells from patients (FIG. 5d), suggesting modulation of the same targets in immortalized and in primary cancer cells.

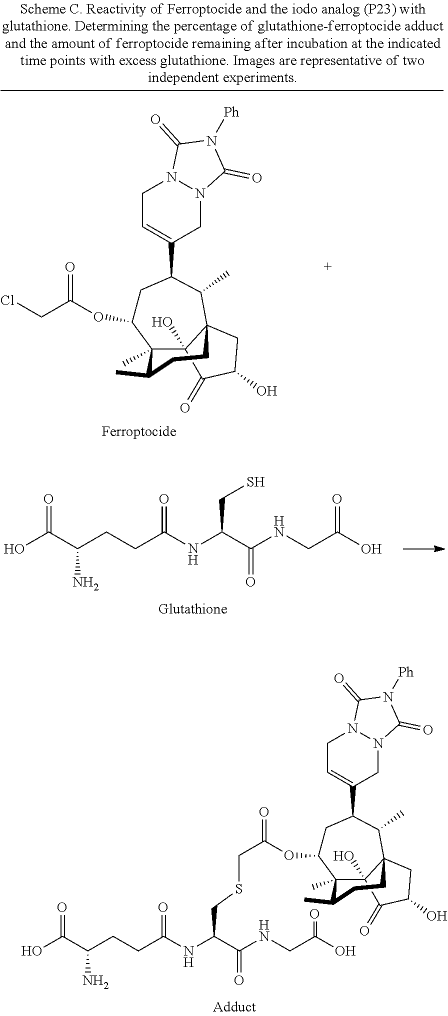

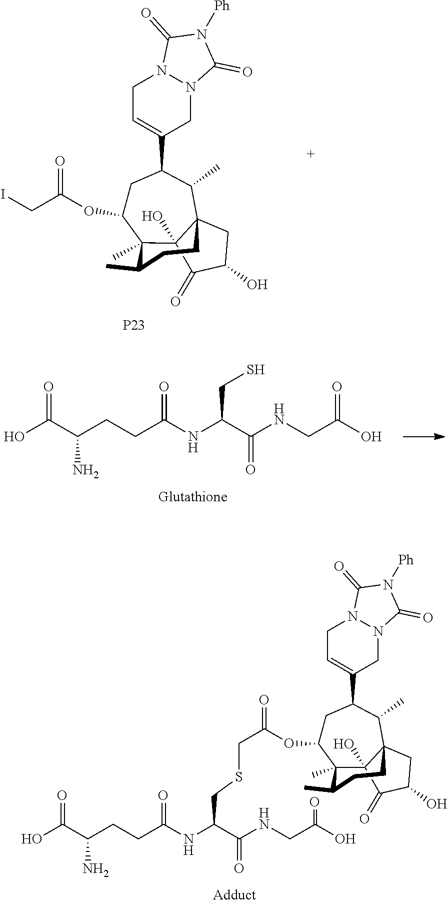

| Time (hr) | GSH-Ferroptocide adduct (%) | Compound remaining (%) |
|---|---|---|
| 0.5 | 4.9 | 95.1 |
| 1 | 18.2 | 81.8 |
| 2 | 33.2 | 66.8 |
| 3 | 38.1 | 61.9 |
| 24 | 72.5 | 27.5 |

In an effort to identify the labeled protein(s), a biotin-streptavidin pulldown (Schematic in FIG. 5e) was performed with the bioactive alkyne compound P29. Briefly, HCT 116 cells were pre-treated with ferroptocide or DMSO, followed by treatment with P29. Upon incorporation of a biotin group using copper-catalyzed azide-alkyne cycloaddition (CuAAC) chemistry, P29-labeled proteins were enriched on streptavidin beads, subjected to an on-bead trypsin digestion and subsequent LC/LC-MS/MS analysis. Protein identities were determined by database searches using the SEQUEST algorithm. Relative quantitation of proteins enriched in ferroptocide and DMSO pre-treated samples was achieved by spectral counting. The DMSO/ferroptocide spectral count ratio provides a relative measure of enriched proteins in the DMSO versus ferroptocide pre-treated sample (FIG. 5f). High-affinity targets from the HCT 116 cell line were then compared to targets identified in ES-2 cells. Based on dual shared enrichment, as well as molecular weights matching the gel bands, nine proteins were selected for follow up characterization (Table 2). Importantly, GPX4 protein was not identified as a target of interest for ferroptocide, with low spectral counts below the cutoff for significance.

TABLE 2
Proteins identified for follow-up characterization based on
shared enrichment in both HCT 116 and ES-2 cell lines, as well
as molecular weights matching the bands observed by gel.

| Protein | MW (kDa) | Fold Change |
|---|---|---|
| TXNRD3 | 76.4 | 7.4 |
| TXNRD1 | 70.8 | 4.9 |
| KEAP1 | 69.7 | 3.1 |
| PDP1 | 61.1 | 3.7 |
| TXNRD2 | 56.4 | 2 |
| PTGES2 | 41.9 | 18 |
| PGLS | 27.5 | 2.7 |
| GSTO1 | 27.5 | 3.6 |
| TXN | 11.7 | 11.1 |

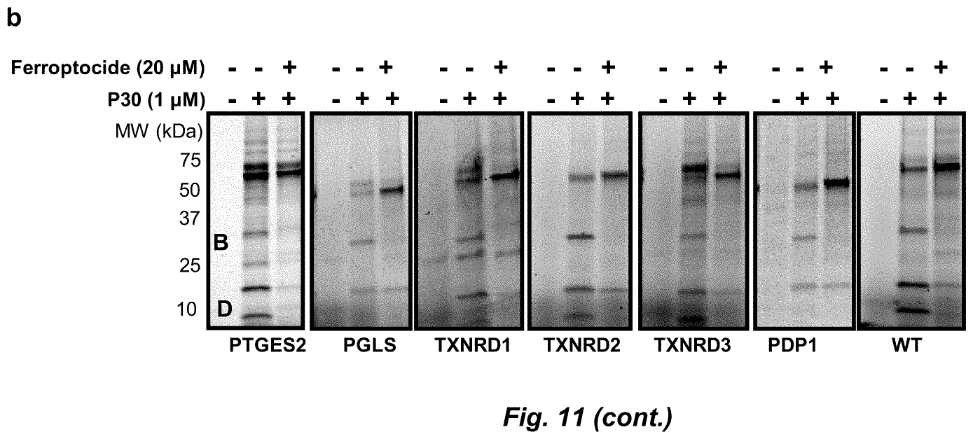

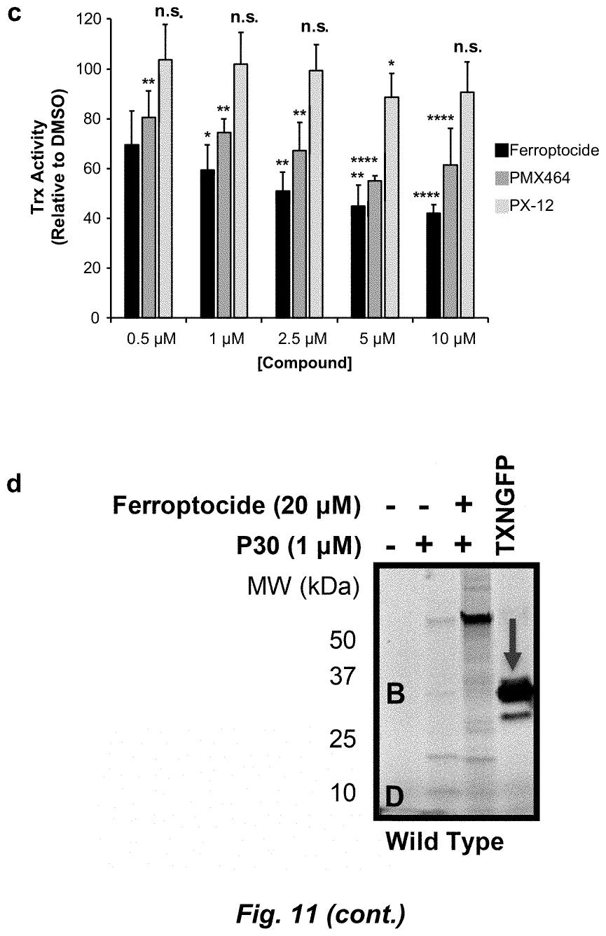

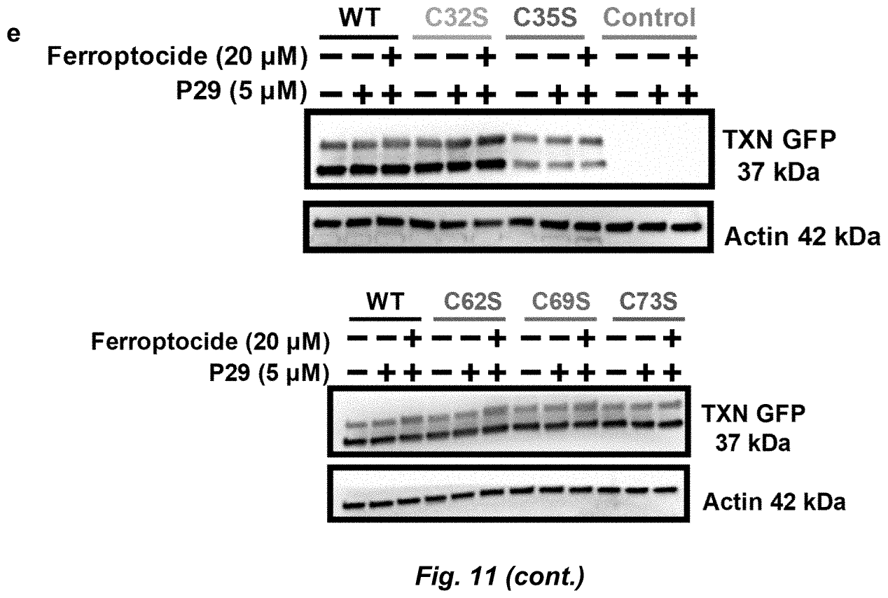

In order to discriminate between on- and off-cytotoxicity-related targets, siRNA and CRISPR Cas9 strategies were employed. KEAP1 and GSTO1 proteins were targeted first due to their molecular weights similar to bands A and B respectively. Upon successful siRNA knockdown of these proteins, an assessment was made of how changes in protein expression affected band labeling in the in-gel fluorescence experiment. Comparison of cells with knockdown targets and wild type cells indicated no change in in-gel fluorescence (FIG. 11a), suggesting that KEAP1 and GSTO1 are off-pathway targets of ferroptocide. CRISPR Cas9 technology was then used to rapidly investigate the remaining targets. We were able to successfully generate isogenic cell line pairs for seven knockout targets, with one target being lethal. Knockout of six targets did not diminish labeling of any of the fluorescent bands indicating that such proteins (PTGES2, PGLS, TXNRD1, TXNRD2, TXNRD3, and PDP1) were not the targets of interest (FIG. 11b), while the lethal target corresponded to that of thioredoxin protein.

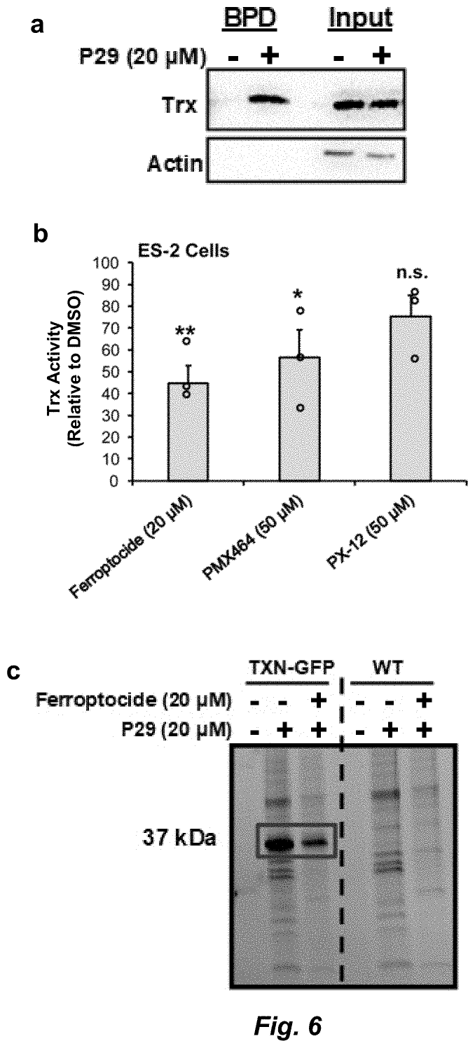

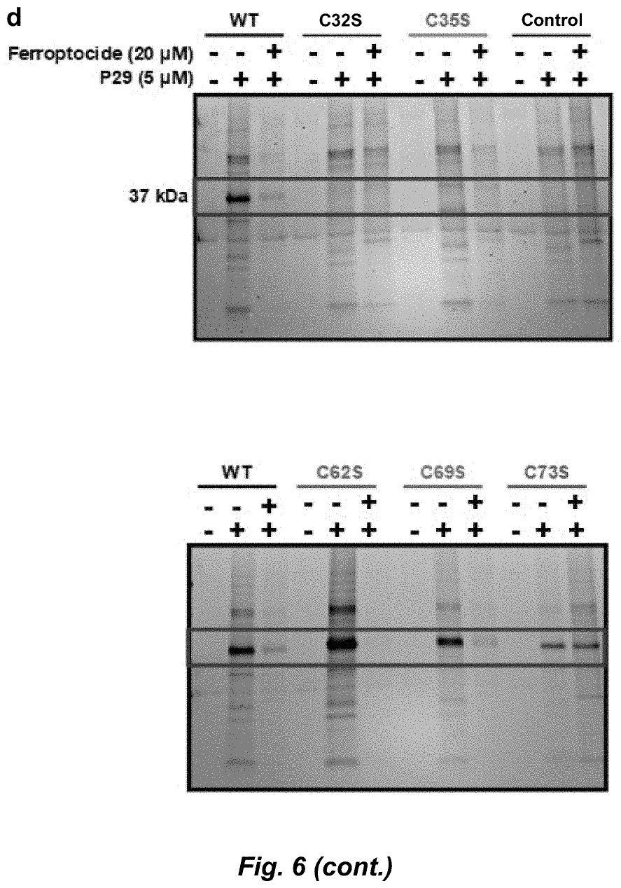

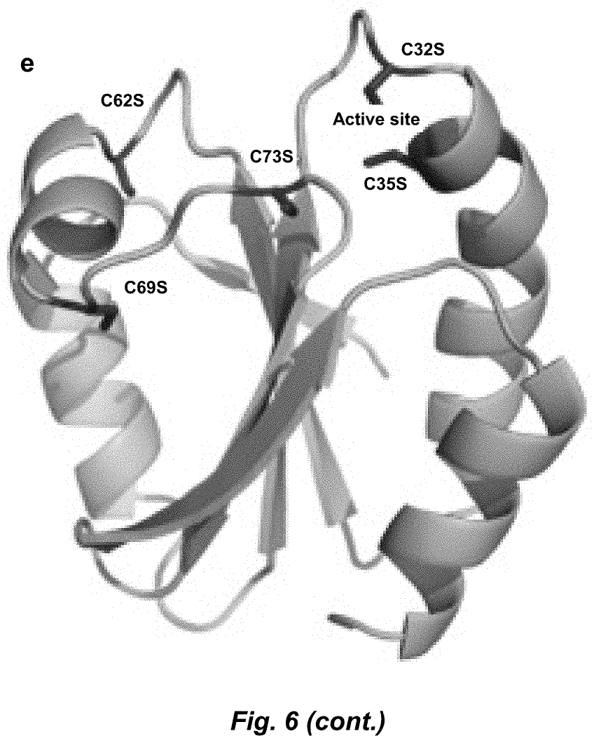

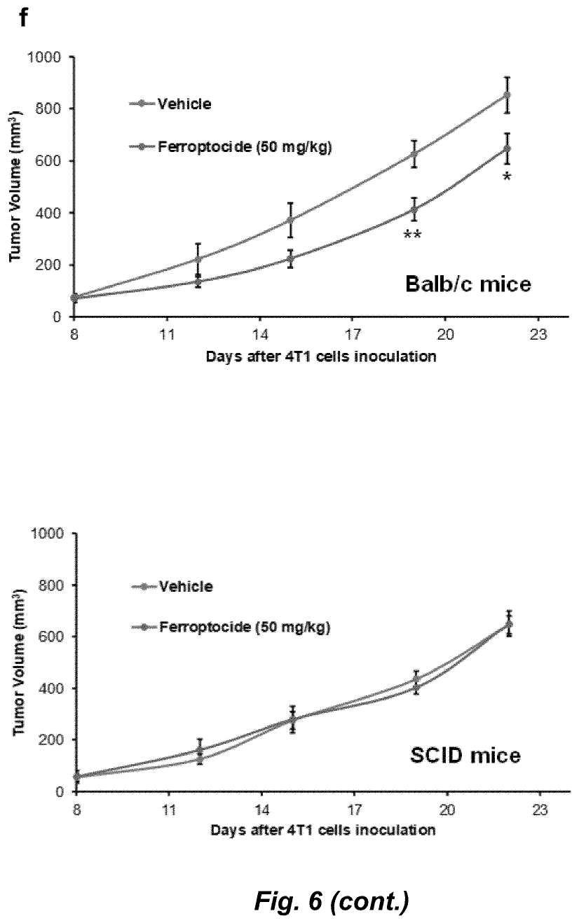

Thioredoxin (TXN) is a 12 kDa ubiquitous oxidoreductase that plays a key role in the thioredoxin antioxidant system comprised of thioredoxin, NADPH, and thioredoxin reductase. Thioredoxin contains 5 cysteines and uses active site cysteines (C32 and C35) to reduce the disulfide bonds of many protein partners such as transcription factors (NF-κB, AP-1, Ref-1), ribonucleotide reductases, peroxiredoxins, and glutathione peroxidases, as well as scavenging of ROS. Treatment of HCT 116 cells with P29, coupled to biotin-streptavidin enrichment followed by immunoblotting yielded a band present only in compound treated sample (FIG. 6a), suggesting that ferroptocide covalently modifies thioredoxin. A thioredoxin activity assay was then employed to assess the ability of ferroptocide to inhibit thioredoxin activity in cell lysate, and this compound significantly reduced the activity of thioredoxin within 30 min of treatment in HCT 116 cells to a greater extent than the two known inhibitors of thioredoxin (PMX464 and PX-12) (FIG. 6b). Dose-response analysis confirmed that ferroptocide is also a more potent thioredoxin inhibitor than PMX464 and PX-12 in a biochemical (in vitro) assay (FIG. 11c).

To further assess the effect of ferroptocide on thioredoxin, thioredoxin fused to GFP (TXN-GFP, 37 kDa) was overexpressed in HCT 116 cells (FIG. 11d). Treatment of these cells with P29, followed by bioconjugation of the orthogonal fluorophore of Cy3, afforded a new band at 37 kDa corresponding to TXN-GFP (FIG. 6c); this new band was competed away upon pretreatment with ferroptocide. To identify the sites of modification of thioredoxin by ferroptocide, site-directed mutagenesis introduced serine mutants of each of the five cysteines of TXN-GFP. The ability of ferroptocide to covalently modify these mutant proteins was assessed after transfection of mutant clones (C32S, C35S, C62S, C69S, and C73S, FIG. 11e) into HCT 116 cells, pretreatment of these cells with ferroptocide followed by alkyne treatment and Cy3 bioconjugation to evaluate fluorescent band labeling. As shown in FIG. 6d, the new band at 37 kDa is not present in the C32S and C35S mutants and has reduced labeling in the C73S mutant, suggesting that ferroptocide is modifying the active site cysteines and the adjacent cysteine 73 of thioredoxin as shown in the crystal structure (FIG. 6e).

Taken together, these studies demonstrate that treatment with ferroptocide modifies critical residues needed for interaction of thioredoxin with its binding partners, and thus inhibiting its activity in cells. This inhibition presumably causes the observed phenotype of rapid ferroptotic cell death. Given that thioredoxin is a key component of a major antioxidant system, it is possible that its modulation renders cells susceptible to oxidative stress that causes lipid peroxidation and other imbalances in cellular processes which eventually lead to ferroptotic cell death; other thioredoxin inhibitors have not been reported to induce ferroptosis.

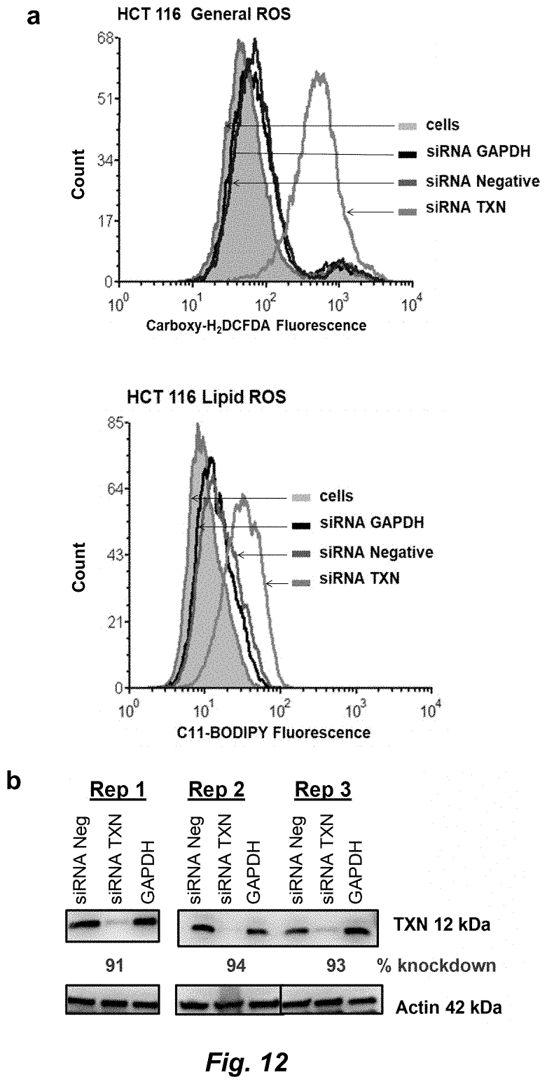

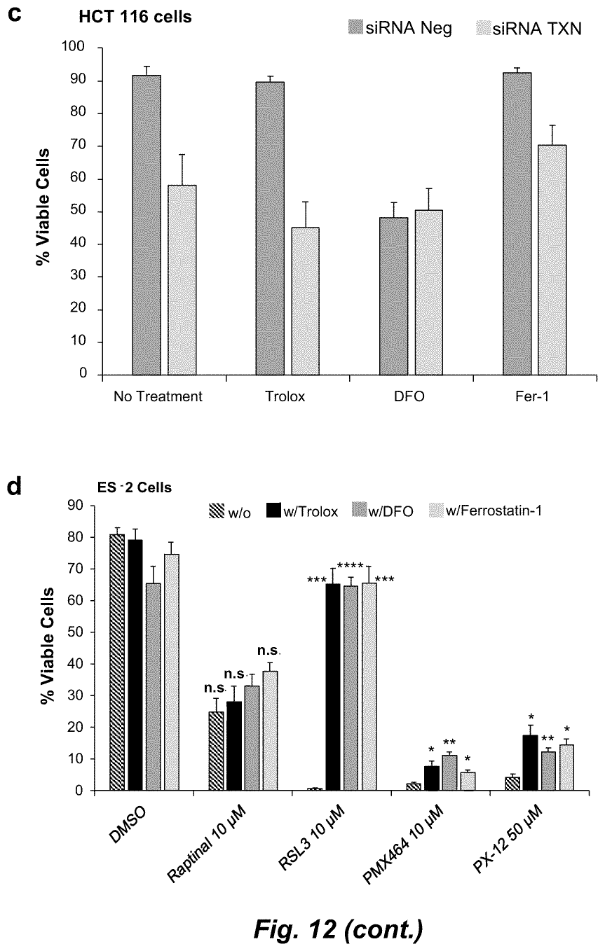

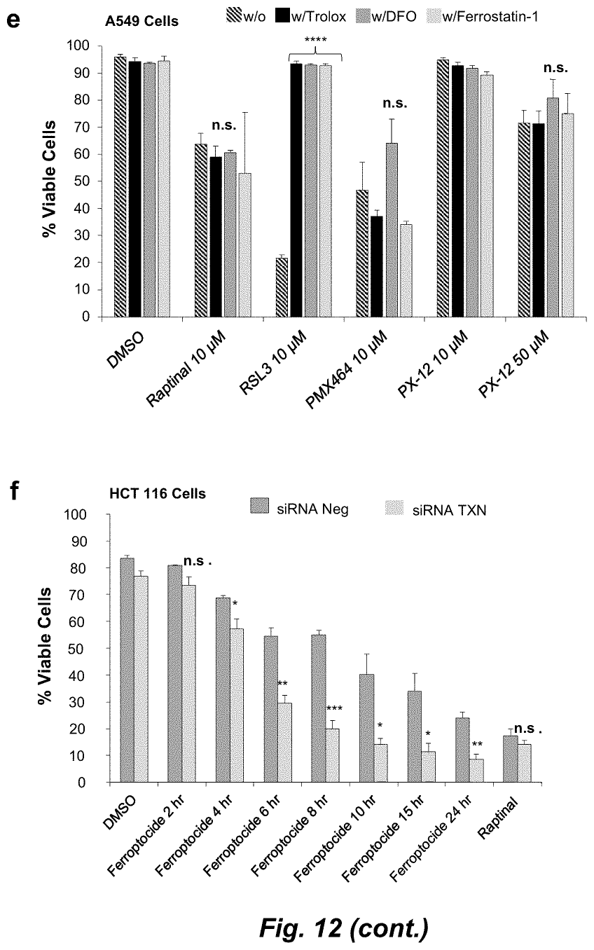

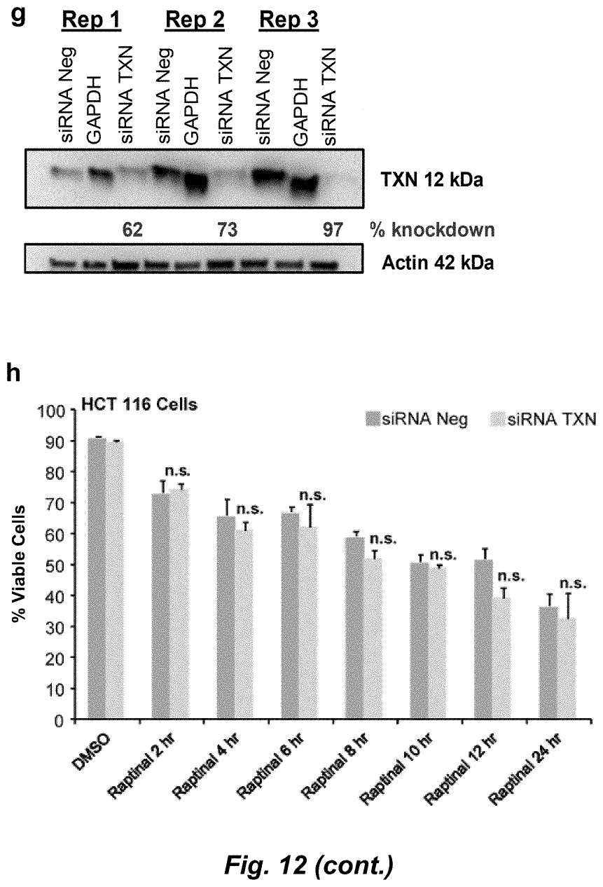

General and lipid ROS levels were monitored upon genetic knockdown of thioredoxin in HCT 116 cells. Knockdown of thioredoxin (siTXN) resulted in massive generation of general ROS and lipid ROS within 72 hr (FIG. 12a, b) consistent with induction of ferroptosis. Pretreatment of HCT 116 cells with ferroptosis inhibitors of trolox and ferrostatin-1 did not protect against siTXN, however, likely due to high ROS levels and long incubation times required for sufficient knockdown of thioredoxin; pretreatment with DFO impaired cell viability even in the control HCT 116 cells transfected with scrambled RNA (siNeg, FIG. 12c). In order to determine if thioredoxin inhibitors cause ferroptotic cell death, protection studies with ferroptosis inhibitors were conducted in two cell lines; minimal protection was observed in ES-2 cells while A549 displayed no change in cell death compared to untreated cells (FIG. 12d, e). These results are unsurprising given that PMX464 and PX-12 are imperfect tool compounds, suspected to engage multiple molecular targets in cells. The siRNA knockdown of thioredoxin sensitizes HCT116 cells to ferroptocide but not Raptinal treatment in a time course study (FIG. 12f), further suggestive of the importance of thioredoxin in ferroptocide-induced cell death. Together, these data support a role of thioredoxin in ferroptocide-induced cell death and ferroptosis.

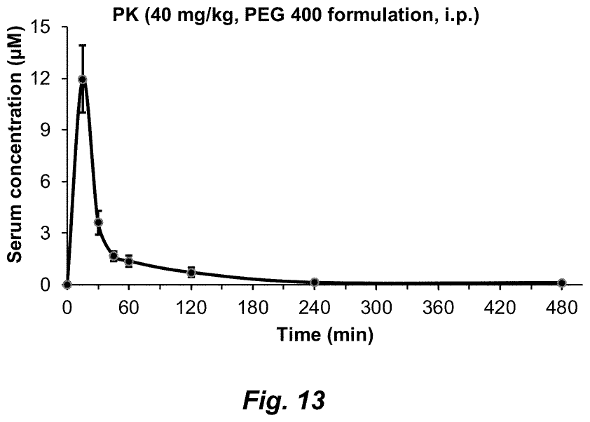

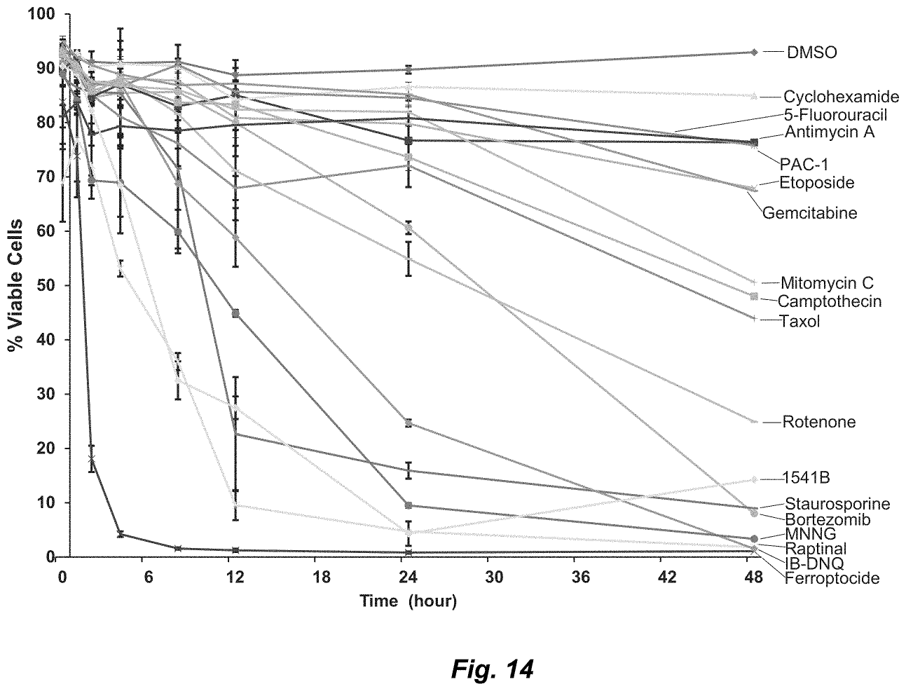

Ferroptocide is an immunostimulatory compound. The non-apoptotic nature of ferroptocide inspired preliminary exploration of its ability to modulate the immune system. Non-apoptotic compounds are attractive anticancer agents, as they can potentially elicit an immune response. Ferroptocide displays some activity in non-cancerous breast (MCF10A) and human skin fibroblast (HFF-1) cells (IC₅₀ of 3.1 and 4.1 μM respectively) but no hemolytic activity, so it is a favorable tool compound to assess in vivo. We investigated the role of the immune system by assessing the efficacy of ferroptocide in a subcutaneous murine model of 4T1 triple negative breast cancer cells in immunocompetent (Balb/c) compared to immunocompromised (SCID) mice. Upon tumor establishment, mice were dosed with 50 mg/kg (twice a week) for five doses before being sacrificed (FIG. 6f). Measurements of tumor volume indicated a 40% tumor growth retardation in compound-treated Balb/c compared to vehicle-treated mice. As shown in FIG. 6f, there was no effect of ferroptocide in immunocompromised mice suggesting that T and B cells play a role in the activity of ferroptocide in vivo. The potency of ferroptocide in this in vivo model is likely limited by its poor pharmacokinetics in mice (FIG. 13 and Table 3).

TABLE 3
Pharmacokinetic (PK) parameters of ferroptocide (40 mg/kg)
in C57BL/6 mice calculated using GraphPad Prism V5.0

| Parameter | Estimate | |
|---|---|---|
| AUC | 422108.3 | min*ng/mL |
| Half-life | 9.96 | min |

Small molecules are powerful tools to investigate protein function and cell death mechanisms. Staurosporine and more recently Raptinal are commonly used to predictably and rapidly induce apoptotic cell death and enable the study of its mechanisms and protein regulators. Selective inhibitors of cell death processes are also extremely valuable, with z-VAD-fmk and Q-VD-OPh widely used to inhibit apoptosis, and necrostatin-1 and ferrostatin-1 used to inhibit necrosis and ferroptosis, respectively.

In contrast to the variety of tool compounds available to induce apoptosis, there are comparatively fewer that can be used to induce ferroptosis, another regulated form of cell death. Erastin and RSL3 are the first reported inducers of ferroptosis, followed by more recent reports of salinomycin, sorafenib, FIN56, and FINO₂. These compounds have been instrumental in the discovery of ferroptosis and elucidation of key ferroptotic regulators (system x_{c}: and glutathione peroxidase 4) and related pathways. However, these compounds typically do not induce quantitative cell death, and lack potent lethality in RAS-mutated cell lines, revealing a need for additional pro-ferroptotic agents. Furthermore, the discovery of other inducers of ferroptosis can uncover additional proteins critical to this cell death process.

Further mechanistic details of the thioredoxin-ferroptosis link remain to be understood, and the possibility that there are alternative targets important for the pro-ferroptotic action of ferroptocide cannot be ruled out.

Head-to-head comparisons of ferroptocide to erastin and RSL3 suggest that ferroptocide may be advantageous, especially for applications requiring induction of rapid and/or quantitative ferroptotic cell death. Furthermore, as ferroptocide-induces a regulated, non-apoptotic mode of cell death, this compound (and possibly other pro-ferroptotic agents) has the potential to synergize with the immune system for the treatment of cancer. Ferroptocide represents a distinct class of ferroptosis inducers and will be an important tool compound for further studies of ferroptosis.

### Pharmaceutical Formulations

The compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, α-ketoglutarate, and β-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The compounds described herein may be systemically administered in combination with a pharmaceutically acceptable vehicle, such as an inert diluent or an assimilable edible carrier. For oral administration, compounds can be enclosed in hard- or soft-shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze-drying techniques, which yield a powder of the active ingredient plus any additional desired ingredient present in the solution.

For topical administration, compounds may be applied in pure form, e.g., when they are liquids. However, it will generally be desirable to administer the active agent to the skin as a composition or formulation, for example, in combination with a dermatologically acceptable carrier, which may be a solid, a liquid, a gel, or the like.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of dermatological compositions for delivering active agents to the skin are known to the art; for example, see U.S. Pat. No. 4,992,478 (Geria), 4,820,508 (Wortzman), 4,608,392 (Jacquet et al.), and 4,559,157 (Smith et al.). Such dermatological compositions can be used in combinations with the compounds described herein where an ingredient of such compositions can optionally be replaced by a compound described herein, or a compound described herein can be added to the composition.

Useful dosages of the compounds described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day.

The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations, such as multiple inhalations from an insufflator or by application of a plurality of drops into the eye.

The non-apoptotic compounds described herein can be effective anti-tumor agents and have higher potency and/or reduced toxicity as compared to proapoptotic compounds. Preferably, compounds of the invention are more potent and less toxic than, for example, Raptinal, and/or avoid a potential site of catabolic metabolism encountered with Raptinal, i.e., have a different metabolic profile than Raptinal.

The invention provides therapeutic methods of treating cancer in a mammal, which involve administering to a mammal having cancer an effective amount of a compound or composition described herein. A mammal includes a primate, human, rodent, canine, feline, bovine, ovine, equine, swine, caprine, bovine and the like. Cancer refers to any various type of malignant neoplasm, for example, colon cancer, breast cancer, melanoma and leukemia, and in general is characterized by an undesirable cellular proliferation, e.g., unregulated growth, lack of differentiation, local tissue invasion, and metastasis.

The ability of a compound of the invention to treat cancer may be determined by using assays well known to the art. For example, the design of treatment protocols, toxicity evaluation, data analysis, quantification of tumor cell kill, and the biological significance of the use of transplantable tumor screens are known. In addition, ability of a compound to treat cancer may be determined using the Tests known to persons of ordinary skill in the art.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Cell Culture Studies

Cells were grown at 37° C. under a humidified 5% CO₂ atmosphere, in a culture medium consisting of high-glucose (Life Technology) DMEM media for Mia PaCa-2, D54, U87, K7-M2, SK-MEL-5, and 3LL cells or RPMI 1640 for ES-2, HCT 116, MDA-MB-231, A549, T47D, B16-F10, and BT-549 cells or McCoy's 5A media for HT29 cells. All media were supplemented with 10% FBS (Gemini), penicillin (50 IU/ml), streptomycin (50 μg/ml) and glutamine (2 mM) (Cellgro). Primary cells were isolated from pleural effusions of metastatic patients at Carle Foundation Hospital (IRB #15149) following a protocol as described previously (Med. Sci. 2, 70-81, (2014)).

### Example 2. Anticancer Screen

40 μL of media was added to each well of a 384-well tissue culture-treated plate. 3×100 nL of compound in DMSO was then pin-transferred from compound storage plates (2 mM stocks) into media-containing wells using the Platemate Plus at the UIUC High Throughput Screening Facility. A 100,000 cells/mL suspension of ES-2 cells was prepared, and 10 μL was added to each well for a final concentration of 1000 cells/well. Doxorubicin (100 μM final) was used as a positive control. Plates were sealed with gas-permeable seals and incubated at 37° C. for 72 h. After incubation, 5 μL of Alamar blue (440 μM resazurin in sterile PBS) was added and allowed to incubate for 3-4 h, until visible color change occurred. Fluorescence was measured in a Molecular Devices SpectraMax 3 (excitation=555 nm, emission=585 nm, emission cutoff=570 nm).

### Example 3. Dose Response (IC₅₀) Curves

To a 384-well plate, 40 μL of 1.25× compound dilution or 1.25% DMSO-containing media was added (final volume of 1% DMSO). Concentrations of compounds tested were 100 μM to 100 nM. On each plate at least 3 technical replicates per compound were performed. Next, 10 μL of a 100,000 cells/mL suspension was added to each well, yielding a final concentration of 1,000 cells/well. To three wells in column 2 was added 1 μL of 10 mM doxorubicin (final concentration of 200 μM) as positive control of cell death. Plates were sealed with gas-permeable seals and incubated at 37° C. for 72 h. At that time, 5 μL of Alamar blue (440 μM resazurin in sterile PBS) was added and plates were incubated for 3-4 hours. Fluorescence was read on a Molecular Devices SpectraMax 3 (excitation=555 nm, emission=585 nm, emission cutoff=570 nm). Wells were normalized to the average of untreated wells (0% cell death). The data were plotted as compound concentration versus percent dead cells and fitted to a logistic-dose response curve using OriginPro 2015 (OriginLab, Northampton, Mass.). The data were generated in triplicate, and IC₅₀ values are reported as the average of three separate experiments along with standard error of the mean.

### Example 4. The Hemolysis Assay

Whole human blood in citrate phosphate dextrose was obtained from Bioreclamation LLC, stored at 4° C. and used before expiration date. 100 μL of whole blood was combined with 500 μL saline (0.9% NaCl) and centrifuged for 5 min at 300×g. The supernatant was carefully removed from the erythrocyte pellet and the liquid was discarded. Washed pellet 3× in 500 μL saline. The erythrocyte pellet was resuspended in 800 μL of Red Blood Cell Buffer (10 mM Na₂HPO₄, 150 mM NaCl, 1 mM MgCl₂, pH 7.4). To a 0.5 mL eppendorf tube or a PCR plate was added 1.0 μL of 30× compound in DMSO and 19 μL RBC Buffer. For negative controls, 1.0 μL DMSO was combined with 19 μL RBC buffer. For positive controls, either 20 μL MilliQ H₂O or 1.0 μL 30% Triton X-100 were combined with 19 μL RBC Buffer. Tubes or plates were briefly centrifuged. Next, 10 μL of washed erythrocyte suspension was added to each tube, then sealed. After incubation at 37° C. for 2 h, samples were centrifuged for 5 min at 300×g, and 20 μL of supernatant was carefully removed and transferred to wells of a clear flat-bottomed 384-well plate. Absorbance was measured at 540 nm. The data were plotted as compound concentration versus percent hemolysis, and fitted to OriginPro (OriginLab, Northampton, Mass.).

### Example 5. Western Botting

ES-2 and HCT 116 cells were treated with compound for the appropriate amount of time. Cells were harvested by centrifugation (3 min, 500×g), washed with PBS and resuspended in RIPA lysis buffer (50 mM Tris, 150 mM NaCl, 1% Triton X-100, 0.5% Na-deoxycholate, 0.1% SDS, pH=7.4) containing 1× protease inhibitor cocktail, and 1 mM PMSF on ice. Whole cell lysates were normalized after determining their protein concentration using a Bradford assay. Samples were resolved in a 4-20% gradient SDS-PAGE gel (Bio-Rad) at 120 V for 1 h, and then transferred to an activated PVDF membrane in Towbin transfer buffer (192 mM glycine, 25 mM Tris-HCl, 20% methanol, pH=8.3) for 2 h at 45 V. Membranes were blocked overnight at 4° C. in 5% milk or bovine serum albumin [BSA] in TBST (as per primary antibody manufacturer's instruction). Membranes were blotted for molecules of interest with primary antibody (1:1000 in 5% BSA in TBST) overnight at 4° C. The bound primary antibodies were detected after using the appropriate secondary HRP conjugated antibodies (1:5000 in TBST) for 1 hour at room temperature. The immunoblots were incubated for 3 min in SuperSignal West Pico Chemiluminescent Substrate (ThermoFisher) mixture before visualization in a ChemiDoc™ Touch Imaging System (Bio-Rad) and processed using ImageLab software (Bio-Rad). Antibodies used: Thioredoxin (Cell Signaling #2429), KEAP-1 (Cell Signaling #8047), GSTO1 (Abcam #129106), Beta-Actin (Cell Signaling #5125), Anti-rabbit IgG HRP linked (Cell Signaling #7074) GAPDH (Cell Signaling #2118), PARP-1 (Cell Signaling #9532)

### Example 6. Cell Viability Via Flow Cytometry

ES-2, HCT 116, Mia PaCa-2, A549, and 4T1 cells (1×10⁵ cells/mL) were plated overnight in 12 well plates, prior to addition of compounds. For protection studies, samples were pre-treated with each protecting agent such as 25 μM Q-VD-OPh, 250 μM trolox, 2 μM ferrostatin-1, 5 mM NAC-1 (neutralized pH), or 100 μM deferoxamine (DFO) for two hours or 1 hr (NAC-1) before compound addition. Cells were incubated for the appropriate times and harvested for flow cytometry analysis. Cell pellets were resuspended in binding buffer (10 mM HEPES, pH 7.4, 140 mM NaCl, 2.5 mM CaCl₂) containing 2 μg/mL propidium iodide and 5 μL/mL Annexin V-FITC conjugate antibody and analyzed for cell viability after gating for forward and side scattering. Ten thousand events were collected per sample in BD LSR II Flow Cytometer (BD Bioscience) and FCS express V6, De Novo software was used to perform experimental analysis.

### Example 7. Confocal Microscopy

ES-2 cells (3×10⁵ cell) were attached overnight in 1.5 mm petri dish plates containing 2 ml of RPMI 1640 media. Prior to imaging, cells were stained for 30 min with Mitotracker Red CMXRoss at 100 nM final concentration. Upon media replacement, samples were treated with 10 μM P30 or 1 μM BODIPY azide for 30 min followed by a PBS wash. PBS or phenol free media was added to each dish and cells were stained with Hoechst 33342 (1 μg/mL). Samples were visualized and analyzed using Carl Zeiss LSM 700.

### Example 8. Confocal Microscopy Competition Studies

ES-2 cells (3×10⁵ cell) were attached overnight in 1.5 mm petri dish plates containing 2 ml of RPMI media. Prior to imaging, cells were stained for 30 min with Mitotracker Red CMXRoss at 100 nM final concentration. Upon media replacement, samples were pre-treated with 3× IC₅₀ of ferroptocide, P27, L1, QQ1 for 30 min followed by treatment with 5 μM P30 for an additional 30 min and a PBS wash. PBS or phenol free media was added to each dish and cells were stained with Hoechst 33342 (1 μg/mL).

Samples were visualized and analyzed using Carl Zeiss LSM 700.

### Example 9. Transmission Electron Microscopy (TEM)

ES-2 cells (2×10⁵ cells/mL) were plated overnight in a 6-well plate. Compounds stocks were loaded in DMSO (0.05% final volume), and cells were incubated for 30 min. Following incubation, cells were pelleted at 500×g, for 3 min, and washed with Hank's buffered salt solution (HBSS). Karnovsky's fixative (0.5 mL) was added to cell pellets upon gently mixing and span at 500×g, 3 min. Samples were stored at 4° C. till analysis. Preparation and imaging of samples was performed by the Center for Microanalysis of Materials of the Frederick Seitz Materials Research Laboratory Central Facilities, University of Illinois. Images of several cells in each sample were taken; displayed images are representative images.

### Example 10. Measurement of Cellular ROS Production

ES-2 and HCT 116 cells (3×10⁵ cells/mL) were plated in 6-well plates. Cells were treated with DMSO, ferroptocide at the indicated concentrations, Etoposide (100 μM), TBHP (100 μM) for 1 hr and 1.5 hr respectively. Cells were washed with HBSS and incubated in the dark for 25 min with 25 μM carboxy-H₂DCFDA probe. Cells were then washed 3× with HBSS and harvested at 1000×g, 3 min. After resuspension in 500 μl HBSS buffer, samples were subjected to flow cytometry to record ten thousand events per sample in FL1 channel in BD LSR II Flow Cytometer (BD Bioscience). FCS express V6 De Novo software was used to generate the histograms.

### Example 11. Measurement of Cellular Lipid ROS Production

ES-2, HCT 116, and 4T1 cells (1×10⁵ cells/mL) were plated in 12-well plates. Cells were pre-treated for 2 hr with 100 μM deferoxamine followed by treatment with DMSO, 10 μM ferroptocide, 10 μM RSL3, or 100 μM TBHP for 1 hr, 1.5 hr and 2 hr respectively. Cells were washed with HBSS and incubated in the dark for 20 min with 5 μM C11-BODIPY probe. Cells were then washed 2× with HBSS and harvested at 1000×g, 3 min. After resuspension in 500 μl HBSS buffer, samples were analyzed with flow cytometry to record ten thousand events per sample in FL1 channel in BD LSR II Flow Cytometer (BD Bioscience). FCS express V6 De Novo software was used to generate histograms.

### Example 12. Measurement of Mitochondrial ROS Production

ES-2 cells (1×10⁵ cells/mL) were plated in 12-well plates and allowed to attach overnight. Cells were treated for 1 hr with DMSO, 10 and 25 μM ferroptocide, 5 μM IB-DNQ, and 10 μM rotenone. Cells were washed with HBSS and incubated in the dark for 10 min with 5 μM MitoSOX Red probe. Cells were then washed 2× with HBSS and harvested at 1000×g, 3 min. After resuspension in 500 μl HBSS buffer, samples were analyzed with flow cytometry to record ten thousand events per sample in the PE channel in BD LSR II Flow Cytometer (BD Bioscience). FCS express V6 De Novo software was used to generate the histogram.

### Example 13. GPX4 LC-MS Based Activity Assay

1 million ES-2 cells/well, 6 well plate were allowed to attach overnight and then treated with DMSO, 10 μM of each compound: ferroptocide, raptinal, and RSL3 for 1 hr. GPX4 enzymatic activity assay was performed with a GPX4 specific substrate, phosphatidylcholine hydroperoxide (PC—OOH) as described previously with minor modifications (Cell 156, 317-331, (2014)). PCOOH was prepared as reported by Roveri and coworkers (Enzymol. Vol. 233 202-212 (Academic Press, 1994)) but using soybean lipoxidase type I-B (L7395, Milipore Sigma). Cells were washed with PBS and lysed by liquid nitrogen freeze-thaw method in the assay buffer (137 mM NaCl, 2.7 mM KCl, 8 mM Na₂HPO₄, 2 mM KH₂PO₄, 1 mM EDTA, 0.1 mM DFO; pH 7.4). Cell lysates were cleared by centrifugation at 14,000 rpm for 10 min at 4° C. Bradford assay was used to determine protein concentration; 200 μg of protein or assay buffer (PCOOH control sample) was mixed with 10 μL PC—OOH in methanol, 5 mM reduced glutathione, and assay buffer to a final reaction volume of 500 μL. The reaction mixture was incubated at 37° C. for 15 mins and extracted with 250 μL chloroform:methanol (2:1) solution. The lipid extract was evaporated under nitrogen and re-dissolved in 100% methanol before injecting into the LC-MS/MS instrument for PCOOH detection.

### Example 14. In Vitro Glutathione Assay

To an eppendorf tube added, 245 μL of 1×PBS, 5 μL of 10 mM DMSO stock of ferroptocide or P23 and 250 μL of reduced GSH (5 mM final concentration). Reactions were vortexed briefly and incubated at 37° C. Adduct formation was monitored at the indicated time points (0, 0.5, 1, 2, 3, 24 hr) by mixing an aliquot of 50 μL from the reaction mixture with 50 μL of MeOH before LC/MS analysis.

### Example 15. In-Gel Fluorescence

HCT 116 cells (3×10⁵ cells/mL) were treated with DMSO or 20 μM ferroptocide for 30 min followed by 30 min treatment with 1 μM P30 in 6-well plates (final DMSO concentration of 0.5%). Cells were harvested by centrifugation (500×g, 3 min), washed with PBS, and resuspended in RIPA lysis buffer (50 mM Tris, 150 mM NaCl, 1% Triton X-100, 0.5% sodium deoxycholate, 0.1% SDS, pH=7.4) containing 1× protease inhibitor cocktail on ice. Protein concentration was determined by the Bradford assay and lysates were stored at −20° C. until further use. Samples were resolved in a 4-20% SDS-PAGE gel (Bio-Rad) at 120 V for 70 min. After PBS washes, each gel was scanned for fluorescence signal using a Molecular Dynamics Typhoon 9400 Multilaser Scanner at the Proteomics Center at UIUC (excitation at 526 nm, green laser, high sensitivity, 530 pmt). Gels were then treated with Coomassie Blue stain (Imperial Stain, ThermoFisher) via shaking for 45 min to stain for the total protein content. Upon proteome labeling, gels were destained for 15 min-overnight on MilliQ H₂O followed by imaging in a Gel Dox XR+ from Bio-Rad (ex. 470 nm, UV High filter).

### Example 16. Biotin-Streptavidin Pulldown for Target ID

ES-2 and HCT 116 (20×10⁶ cells/flask) were plated in T175 flasks and treated with DMSO or 20 M ferroptocide for 1 hr followed by treatment with 20 μM P29 for 1 hr. Cells were lysed via sonication (6500 g, 4 min) in DPBS and the soluble proteome was isolated after ultrasonication (45000 g, 45 min). Bradford assay was used to determine protein concentration. The soluble protein lysates in DPBS (pH 7.4) (500 μL, 2 mg/mL) were treated with biotin-azide (100 μM, 50× stock in DMSO), TCEP (1 mM, 50× fresh stock in water), TBTA ligand (100 μM, 17× stock in DMSO:t-butanol=1:4), and copper(II) sulfate (1 mM, 50× stock in water) followed by incubation at r.t. for 1 hr. Samples were centrifuged (6500 g, 4 min, 4° C.) and the supernatant was discarded. The pellets were resuspended in cold methanol by sonication (2×) and then were solubilized in DPBS containing 1.2% SDS via sonication and heating (90° C., 5 min). A final SDS concentration of 0.2% was achieved after addition of 5 mL of DPBS to the SDS-solubilized proteome samples. The solution was incubated overnight at 4° C. with 100 μL of streptavidin-agarose beads (ThermoFisher, washed 3× with DPBS to remove storage buffer). Samples were rotated at 22° C. for 2 hr before being washed by 5 mL 0.2% SDS/DPBS, 3×5 mL DPBS, and 3×5 mL water. The beads were pelleted by centrifugation (1400×g, 3 min) between washes.

### Example 17. On Bead Trypsin Digestion

The washed beads were suspended in 500 μL of 6 M urea/DPBS and 10 mM DTT (from 20× stock in water) and heated for 20 min on a 65° C. heat block. Upon addition of iodoacetamide (20 mM from 50× stock in water), samples were allowed to react at 37° C. for 30 min while shaking. Following reduction and alkylation, the beads were pelleted by centrifugation and resuspended in 200 μL of 2 M urea/DPBS, 1 mM CaCl₂) (100× stock in water), and sequencing-grade trypsin (2 μg). The bead digestion occurred overnight at 37° C. while shaking. Next day, the beads were pelleted by centrifugation and washed with 2×50 μL water. The washes were combined with the supernatant from the trypsin digestion step, and after addition of formic acid (15 μL) to each sample, they were stored at −20° C. until mass spectrometry analysis.

### Example 18. Liquid Chromatography—Mass Spectrometry Analysis

LC/LC-MS/MS analysis was performed on an LTQ-Orbitrap Discovery mass spectrometer (ThermoFisher) coupled to an Agilent 1200 series HPLC. Peptide digests were pressure loaded onto a 250 m fused silica desalting column packed with 4 cm of Aqua C18 reverse phase resin (Phenomenex). The peptides were eluted onto a biphasic column (100 m fused silica with a 5, tip, packed with 10 cm C18 and 4 cm Partisphere strong cation exchange resin (SCX, Whatman) using a gradient 5-100% Buffer B in Buffer A (Buffer A: 95% water, 5% acetonitrile, 0.1% formic acid; Buffer B: 20% water, 80% acetonitrile, 0.1% formic acid). The peptides were then eluted from the SCX onto the C18 resin and into the mass spectrometer using 4 salt steps previously described (Nat. Protoc. 2, 1414-1425, (2007)). The flow rate through the column was set to ˜0.25 μL/min and the spray voltage was set to 2.75 kV. One full MS scan (FTMS) (400-1800 MW) was followed by 8 data dependent scans (ITMS) of the n^{th} most intense ions. The tandem MS data were searched using the SEQUEST algorithm using a concatenated target/decoy variant of the human UniProt database. A static modification of +57.02146 on cysteine was specified to account for alkylation by iodoacetamide. MS2 spectra matches were assembled into protein identifications and filtered using DTASelect2.0 to generate a list of protein hits with a peptide false-discovery rate of 5%. The mass spectrometry proteomics data have been deposited to the ProteomeXchange Consortium via the PRIDE [1] partner repository with the dataset identifier PXD012805.

### Example 19. Generation of CRISPR-Mediated Knockout HCT 116 Cell Lines

TXNRD3, TXNRD2, TXNRD1, PTGES2, PGLS, PDP1 KO cell lines were generated using CRISPR Cas9 nature protocol. In brief, sgRNAs targeting TXNRD3, TXNRD2, TXNRD1, PTGES2, PGLS, PDP1, and TXN (described in Table 4A below) were designed, amplified, and cloned into P2-gRNA (from Perez lab) in a one-pot reaction as described previously (Methods and Protocols 235-250 (Springer New York, 2017)). Plasmid DNA was isolated using the QIAminiprep kit (QIAGEN cat #27104) according to manufacturer's recommendation. HCT 116 cells (3×10⁵ cells/mL) were transfected for 48 hr with specific plasmids (gene of interest [GOI] sgRNA, empty vector GFP, TV puro, TV hygro, Cas9, pAB059) using lipofectamine transfection agent following manufactures protocol. After 7-10 days of puromycin and hygromycin double selection, clonal cells were isolated, expanded and analyzed for KO efficiency of GOI using a three-way PCR as described previously (ACS Synth. Biol. 5, 582-588, (2016)) using GOI fwd primer, GOI rev primer, and GFP rev primer ww443. Desired clonal cells were used for downstream analysis as described in the in-gel fluorescence experiment above.

TABLE 4A
Sequence listing

| | | SEQ ID |
|---|---|---|
| sgRNA | Sequence | NO: |
| TXNRD3 Fwd | CACCGACACTCAATCGTGCCCTCAC | 1 |
| TXNRD3 Rev | AAACGTGAGGGCACGATTGAGTGTC | 2 |
| TXND2 Fwd | CACCGATTAGGAGGGCGCTTCCGG | 3 |
| TXNRD2 Rev | AAACCCGGAAGCGCCCTCCTAATC | 4 |
| TXNRD1 Fwd | CACCGAGAACGGCGATGGCCGCCGT | 5 |
| TXNRD1 Rev | AAACACGGCGGCCATCGCCGTTCTC | 6 |
| PTGES2 Fwd | CACCGCGCCGTGTGGTACAGCCCC | 7 |
| PTGES2 Rev | AAACGGGGCTGTACCACACGGCGC | 8 |
| PGLS Fwd | CACCGCGTGCTCTCGGCGTGATCGA | 9 |
| PGLS Rev | AAACTCGATCACGCCGAGAGCACGC | 10 |
| PDP1 Fwd | CACCGAACGATTCTTCCCGACGAGG | 11 |
| PDP1 Rev | AACCCTCGTCGGGAAGAATCGTTC | 12 |
| TXN Fwd | CACCGTACTTCAAGGAATATCACGT | 13 |
| TXN Rev | AAACACGTGATATTCCTTGAAGTAC | 14 |
| | | SEQ ID |
| Primer | Sequence | NO: |
| TXNRD3 Fwd | TCA GAT TGC AGC GGG ATG TT | 15 |
| TXNRD3 Rev | GCT GTT AAA AAC CGG CCT CC | 16 |
| TXNRD2 Fwd | CCT ATC CCA GTG TTC CAC CC | 17 |
| TXNRD2 Rev | GAG ACC ACA GGT GCA GTC AG | 18 |
| TXNRD1 Fwd | TGG CCT GTG GGA CTT AAA TGG | 19 |
| TXNRD1 Rev | CGA GTA GCT GCG ACT ACA GG | 20 |
| PTGES2 Fwd | CTG CAG CTC GTA AGG GGA G | 21 |
| PTGES2 Rev | GGG GTG AGC CTA TAG TCC CA | 22 |
| PGLS Fwd | TTC TCG AGT TCC CAG GAG CT | 23 |
| PGLS Rev | TTG GCA TGA TAC CCA GTG GC | 24 |
| PDP1 exon 3 | CTC CCC TCC CAC TCG TCA | 25 |
| Fwd | | |
| PDP1 exon 3 | CCT CCC TGG AGC TCA CTC T | 26 |
| Rev | | |
| TXN Fwd | CCC ACA TTG AAA CAT GGG CC | 27 |
| TXN Rev | TGG TGA CTC CAT CAA GCC TAT | 28 |
| | G | |
| ww443 GFP | TGCCCTTGTCTTGTAGTTTCC | 29 |
| rev | | |
| Ab299 | CCTGACTGTGGGCTTGTAT | 30 |
| PuroR1 | | |
| Ab297 | GCGGTGAGTTCAGGCTTTTT | 31 |
| HygroR1 | | |
| T7 Primer | TAATACGACTCACTATAGGG | 32 |

### Example 20. siRNA Transfection

HCT 116 cells (1×10⁵ cells/mL) were transfected for 72 hr with 5 nM of GSTO1 (silencer select #s18089, ThermoFisher), KEAP1 (silencer select #s18981 ThermoFisher), GAPDH as positive control (silencer select #4390849, ThermoFisher) or negative control siRNA (Qiagen, #1027280) following the Interferin polyplus transfection protocol. Cells were then pre-treated for 30 min with DMSO or 20 μM ferroptocide followed by 30 min treatment with 1 μM P30. Cells were harvested, washed and subjected to in gel-fluorescence studies as described above. siRNA transfection efficiency was assessed via western blot analysis.

### Example 21. Studies with siRNA Transfection of Thioredoxin

HCT 116 cells (1×10⁵ cells/mL) were transfected for 72 hr or 48 hr with 5 nM of TXN (silencer select #s1 4390824, ThermoFisher), GAPDH as positive control (silencer select #4390849, ThermoFisher) or negative control siRNA (Qiagen, #1027280) following the Interferin polyplus transfection protocol. Cells were then used to monitor general and lipid ROS accumulation or time course cell viability studies respectively using flow cytometry as described in this manuscript. In the time course studies, cells were treated with DMSO, 10 μM ferroptocide, 10 and 2.5 μM Raptinal at the indicated time points, followed by AV/PI analysis.

In the case of protection studies, same transfection protocol was followed (72 hr, 5 nM siTXN, siGAPDH, siNegative) after pre-treatment of HCT 116 cells with 250 μM trolox, 100 μM deferoxamine (DFO), or 2 μM ferrostatin-1 for two hours. siRNA transfection efficiency for all experiments was assessed via western blot analysis.

### Example 22. Thioredoxin Validation Via Pulldown

HCT 116 cells (1×10⁶ cells/flask) in T25 flasks were treated with DMSO or 20 μM ferroptocide for 30 min followed by a 60 min treatment with 20 μM P29. Cells were lysed via sonication in PBS and the soluble proteome (100 μg) was subjected to click reactions with 400 μM biotin-azide, 1 mM TCEP freshly made, 100 μM THPTA, 1 mM CuSO₄ at r.t. for 60 min. Samples were quenched with 70% cold ethanol, centrifuged at 6500×g, 4 min and supernatant was discarded. The pellets were solubilized in 1.2% SDS/PBS solution by heating (90° C., 5 min). Pierce streptavidin magnetic beads (50 μL) were activated per manufacturer's recommendation and added to each sample in addition to 500 μL of PBS to achieve a final 0.2% SDS concentration. After rotating for 12 hr at 4° C., proteins of interest were eluted with 2× SDS laemmli dye. Proteins were resolved in 4-20% gradient SDS gel (120V, 60 min), transferred in activated immunoblot membranes, blocked in 5% BSA TBS-T, and incubated overnight with thioredoxin antibody (1:1000). Membranes labeled with the primary antibody thioredoxin (Cell Signaling #229) and Beta-actin (Cell Signaling #5125) were then incubated with anti-rabbit HRP-conjugated antibody (Cell signaling #7074) diluted 1:3,000 for 60 min and washed with TBS-T for 2×10 min. Membranes were visualized using the Pico Plus Chemiluminescence Kit (#3477 ThermoFisher); images were captured using a ChemiDoc™ Touch Imaging System (Bio-Rad) and processed using ImageLab software (Bio-Rad).

### Example 23. TXNGFP and TXNGFP Mutants In-Gel Fluorescence

HCT 116 cells (3×10⁵ cells/mL) at 80% confluency in 6 well plates, were transfected with 2.5 μg TXNGFP, empty vector GFP, or cysteine to serine mutant plasmid DNA for 24 hr (jetPRIME, Polyplus). Cells were then pre-treated with DMSO or 20 μM ferroptocide followed by treatment with 20 μM P29 for 1 hr. 50 μg of cell lysate was subjected to click conditions (freshly made 1 mM TCEP, 100 μM THPTA, 1 mM CuSO₄) using 20 μM Cy3 azide fluorophore at r.t. for 1 hr. Reaction was stopped with 50 μL 2× SDS and proteins were resolved at 120 V for 1 hr. After a PBS wash, gels were scanned for a fluorescence signal using a Molecular Dynamics Typhoon 9400 Multi-laser Scanner (excitation at 526 nm, green laser, high sensitivity, 530 pmt).

### Example 24. Site-Directed Mutagenesis

New England Biolabs (NEB) Q5 Site-directed mutagenesis kit protocol (#E0554) was used to generate cysteine to serine mutants for each of the five cysteines of thioredoxin in NEB highly efficient chemically competent cells by employing the primers shown below (Table 4B). Plasmid DNA was isolated using the QIAminiprep kit (QIAGEN cat #27104), submitted for sequencing at Roy J. Carver Biotechnology center, and found to contain only the desired mutant. HCT 116 cells (3×10⁵ cells/mL) were platted in 6 well plates. Upon reaching 80% confluency, cells were transfected for 24 hr with 1 μg plasmid DNA of each mutant or empty GFP vector. Next day, cells were treated with DMSO or 20 μM ferroptocide for 30 min, followed by 60 min treatment with 20 μM P29. Cells were harvested, washed 2× with PBS and lysed in RIPA buffer. Samples (50 μg) were then subjected to click chemistry conditions with Cy3-azide and in-gel fluorescence studies as described above.

TABLE 4B
Sequence listing

| | | SEQ ID |
|---|---|---|
| Primer | Sequence | NO: |
| C32Fwd | AGCCACGTGGTCTGGGCCTTGCA | 33 |
| C32Rev | GAGAAGTCAACTACTACAAGTTTATCACCTGC | 34 |
| C35Fwd | GTGTGGGCCTTCCAAAATGATCAAG | 35 |
| C35Rev | CACGTGGCTGAGAAGTCA | 36 |
| C62Fwd | TGTGGATGACTCTCAGGATGTTG | 37 |
| C62Rev | TCTACTTCAAGGAATATCAC | 38 |
| C69Fwd | TGCTTCAGAGTCTGAAGTCAAATG | 39 |
| C69Rev | ACATCCTGACAGTCATCC | 40 |
| C73Fwd | TGAAGTCAAATCCATGCCAACATTC | 41 |
| C73Rev | CACTCTGAAGCAACATCC | 42 |

### Example 25. Thioredoxin Activity Assay

HCT 116 cells (1×10⁶ cells/flask) were treated with DMSO, 10 μM ferroptocide, 50 μM PMX464, or 50 μM PX-12 for 30 min. Cells were harvested by centrifugation, resuspended in the assay buffer and lysed via sonication. For each condition, 20 μg of cell lysate was used to measure the activity thioredoxin activity following manufacturer' protocol (Cayman Chemical, Fluorescent Thioredoxin Activity Assay kit #20039) in a 96-well black-walled plate. For in vitro studies, human thioredoxin (10 μL of 0.2 μM solution) was used in addition to thioredoxin reductase (10 μL of 1.0 μM solution), 1 μl of DMSO, ferroptocide, PMX44, or PX-12 at indicated concentrations, 5 μL of NADPH (diluted according to manufacturer's instructions), and assay buffer to a final volume of 75 μL. The plate was incubated at 37° C. for 30 min followed by immediate addition of 20 μL fluorescent substrate per each well (diluted as instructed in the assay kit). Fluorescence was monitored over 1 hr at 520 nm after excitation at 480 nm in a SpectraMax M3 (Molecular Devices) instrument at 37° C.

### Example 26. RNA Sequencing

Total RNA was extracted by RNeasy Kit (QIAGEN) and digested with DNase (QIAGEN) from n=2 samples per condition (DMSO, 10 μM ferroptocide cells treated for 6 hr). RNA quality was assessed with a 2100 Agilent Bioanalyzer prior to library preparation. The RNAseq libraries were prepared using the TruSeq Stranded mRNAseq Sample Prep kit (version 1) following manufacturer's instruction (Illumina). Libraries were then quantified, pooled, and sequenced by single-end 150 base pairs using the Illumina HiSeq 4000 platform at the Roy J. Carver Biotechnology center. FASTQ files were generated and demultiplexed with the bcl2FASTQ v2.17.1.14 Conversion Software (Illumina). Libraries were sequenced at an average depth of 40-50 million reads per sample. Trimmomatic (v0.36) was utilized to remove sequencing adapters, low-quality bases (PHRED score <28), and reads less than 30 bases in length. Pseudo-alignment and transcript-level counting to NCBI's GRCh38.p11 transcriptome was then performed with Salmon (v 0.8.2) in quasi-mapping mode while correcting for sequence-specific and GC biases, and generating 30 bootstraps. Counts summarized to the gene level (NCBI annotation release 108) using tximport (v 1.6.0) and the “lengthScaledTPM” method. The counts were normalized using the TMM method from edgeR (v 3.20.5) and then transformed to log 2 counts per million (log CPM) with prior.count=3. Genes without at least log CPM>log 2(0.5) in at least 3 samples were filtered out, leaving 16,743 genes for differential expression testing. TMM normalization was re-done after filtering and then limma's (v 3.34.5) voom method was used to find differentially expressed genes for the pairwise comparisons of treatment vs. control in A549, and treatment vs. control in HT29 (batch 2 and 3, 8 samples total, 6 hr), and the interaction between treatment and cell line. P values were adjusted for multiple hypothesis testing with the Benjamini-Hochberg method to generate false discovery rates. Gene set enrichment analysis was done using ESGEA (v 1.6.1) for the Gene Ontology BP, CC and MF gene sets, KEGG pathway gene sets, and Pathway gene sets. The RNA sequencing data have been deposited to the GEO repository with the accession number GSE126868.

### Example 27. Animal Studies. MTD of Ferroptocide

The protocol was approved by the IACUC at the University of Illinois at Urbana-Champaign (Protocol Number: 14173). These studies used 10- to 12-week-old female C57BL/6 mice, that were purchased from Charles River. Ferroptocide was formulated in 100% PEG400 and given by i.p. All mice were monitored over the course of the study for signs of toxicity and weight loss.

### Example 28. Pharmacokinetic Assessment of Ferroptocide

The protocol was approved by the IACUC at the University of Illinois at Urbana-Champaign (Protocol Number: 14173). In these studies, 10- to 12-week-old female C57BL/6 mice (purchased from Charles River) were used. Ferroptocide was formulated in 100% PEG400. Mice were treated with ferroptocide (40 mg/kg) via i.p. with three mice per time point (15, 30, 45, 60, 120, 240, 480, and 1440 min). At specific time points, mice were sacrificed, and blood was collected, centrifuged; the serum was frozen at −80° C. until analysis. The proteins in a 50 μL aliquot of serum were precipitated by the addition of 50 μL acetonitrile and the sample was centrifuged to remove the proteins. Serum concentrations of ferroptocide were determined by reverse phase HPLC (Shimadzu Corporation, Japan). PK parameters were determined using GraphPad Prism Version 5.00 for Windows.

### Example 29. 4T1 Syngeneic Model

The protocol was approved by the IACUC at the University of Illinois at Urbana-Champaign (Protocol Number: 17192). 9-week old female Balb/C or SCID mice (Charles River) were lightly sedated with i.p. xylazine/ketamine/saline solution. Following sedation, 4T1 murine breast cancer cells suspended in chilled HBSS (100 μL of 4×10⁶ cells/mL) were injected subcutaneously into the right flank of shaved and sedated mice using an insulin syringe. On day 8 after inoculation, mice were randomized with 7 mice per group for vehicle or ferroptocide treatment. Vehicle (PEG400) or ferroptocide (50 mg/kg) was administered intraperitoneally as a PEG400 solution twice a week for 5 times. Tumor measurements were performed every 3 or 4 days using a caliper and tumor volume was calculated using the equation (0.5×l×w²). On day 23 after the 4T1 cells inoculation, mice were sacrificed. Tumors were then surgically removed, and their mass was measured.

### Example 30. Statistical Analysis. All Statistical Analysis was Performed Using an Unpaired, Two-Tailed Student's t Test where p<0.05 Values were Considered Statistically Significant

### Example 31. Compounds Produced by Ring Distortion of Pleuromutilin (P1-P18, P32-P34)

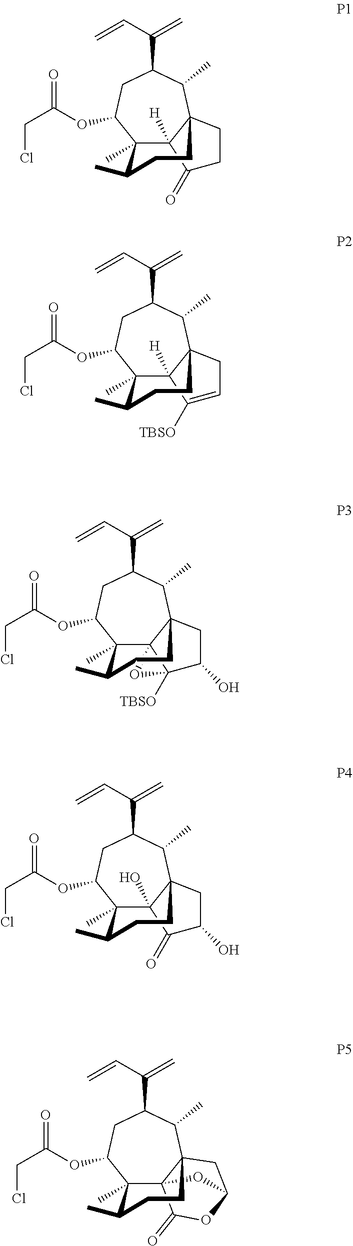

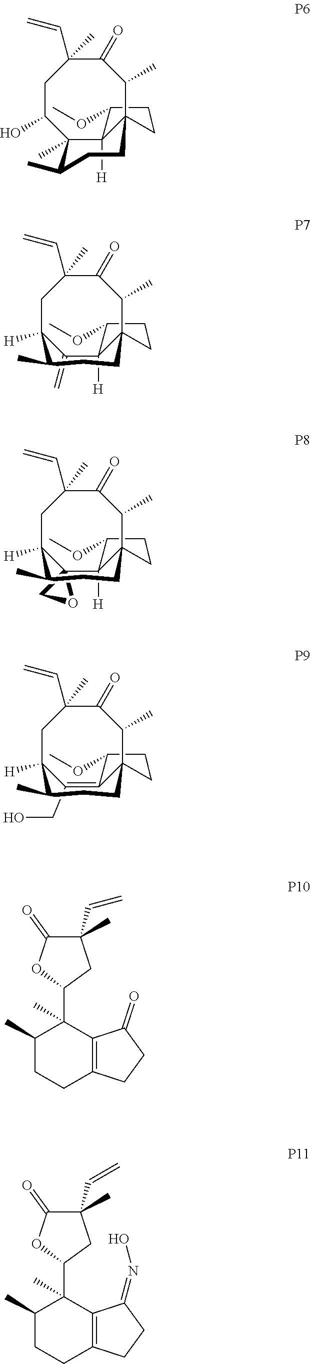

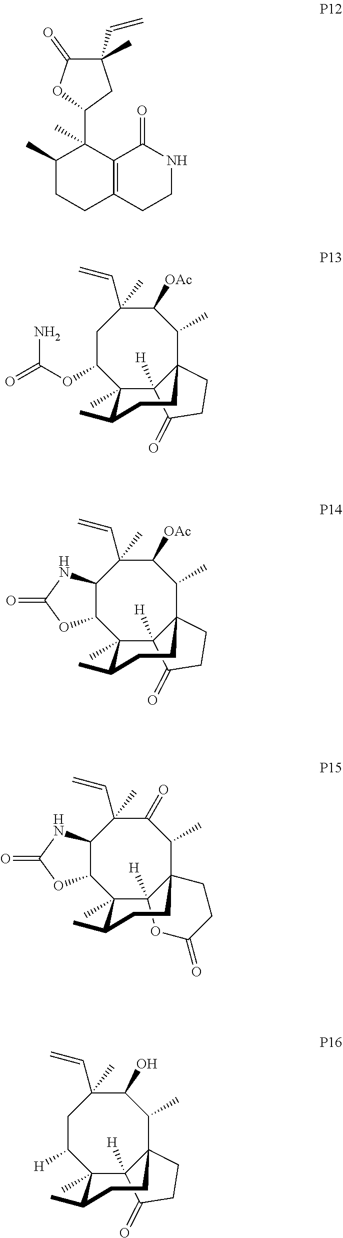

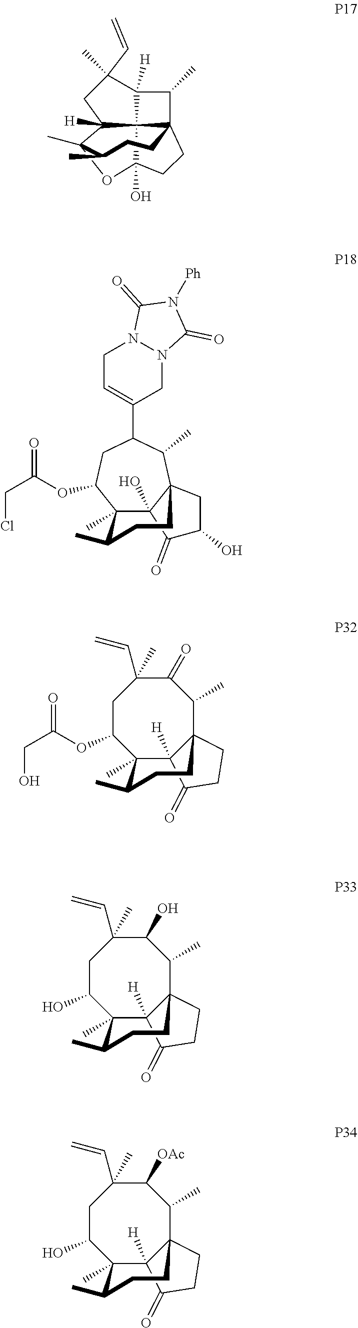

### Example 32. Overview of Synthetic Procedures

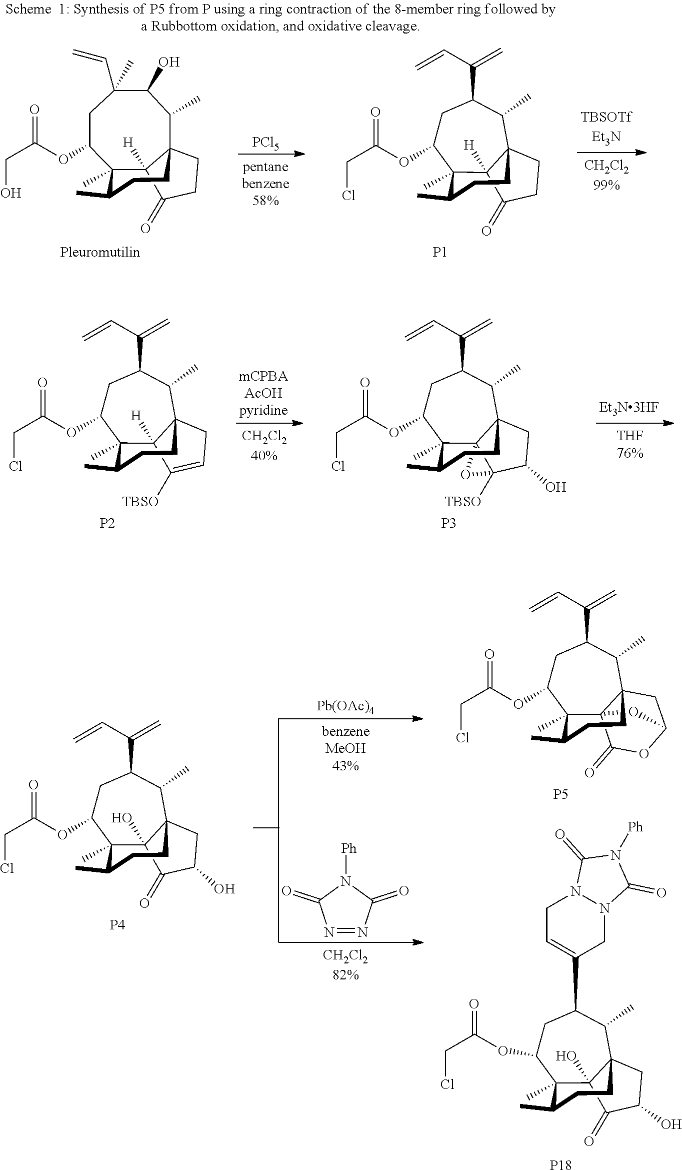

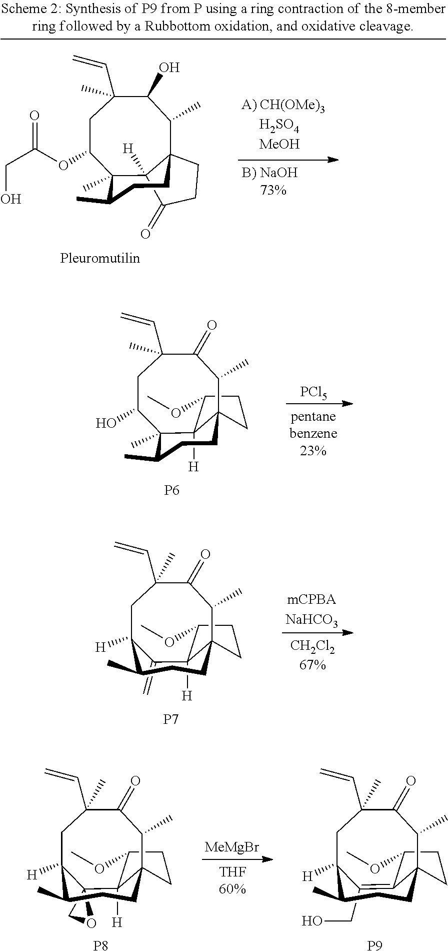

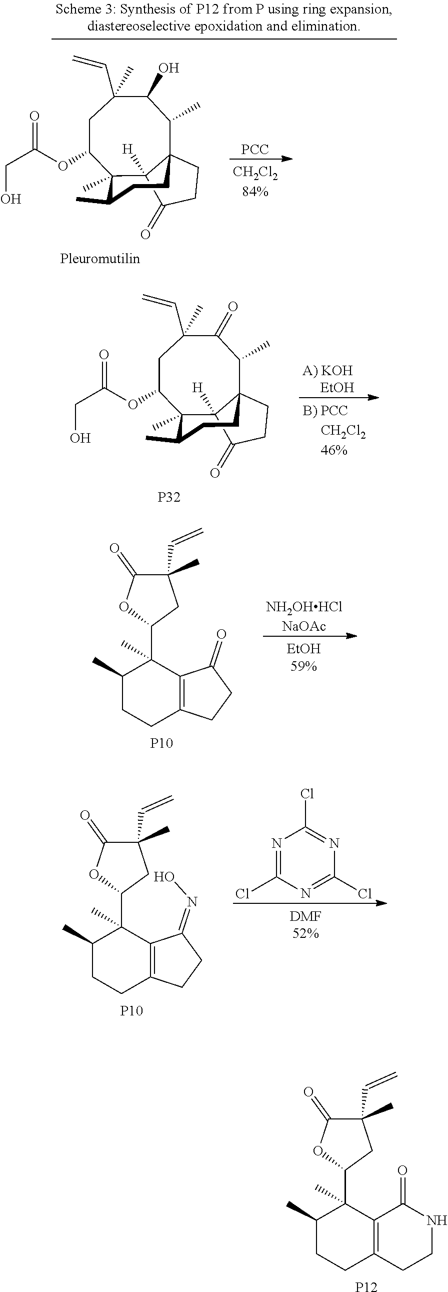

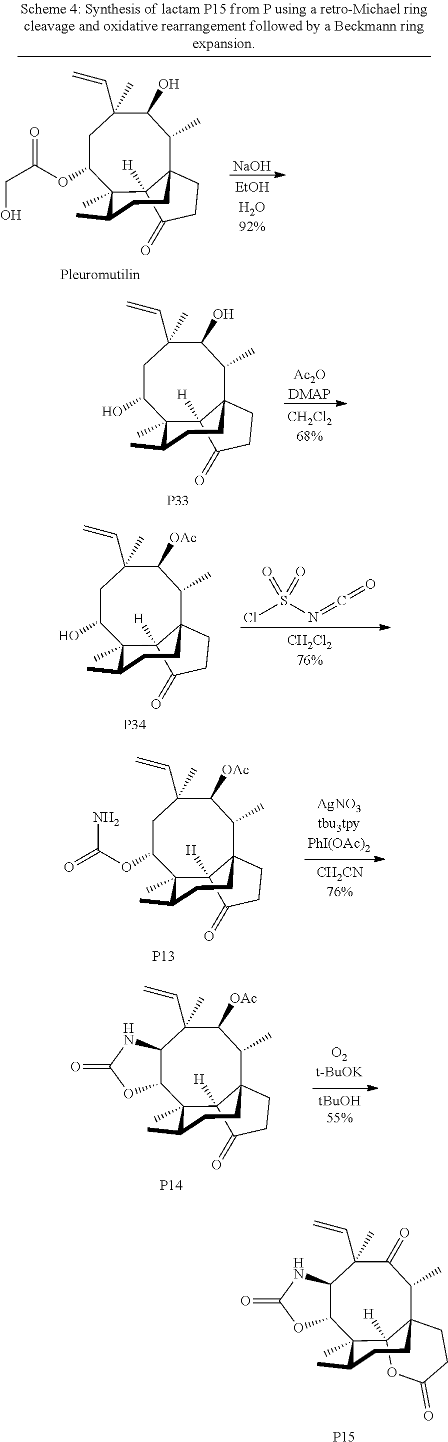

### Example 33. Overview of SAR Derivatives

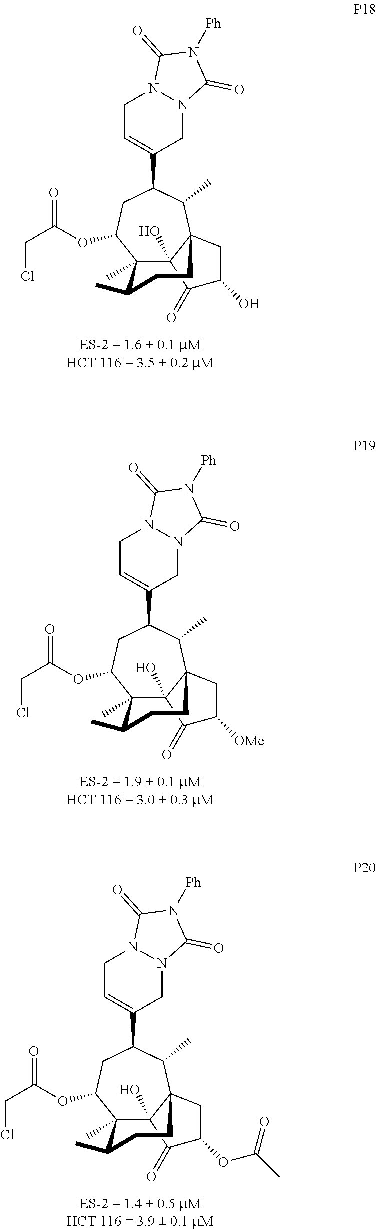

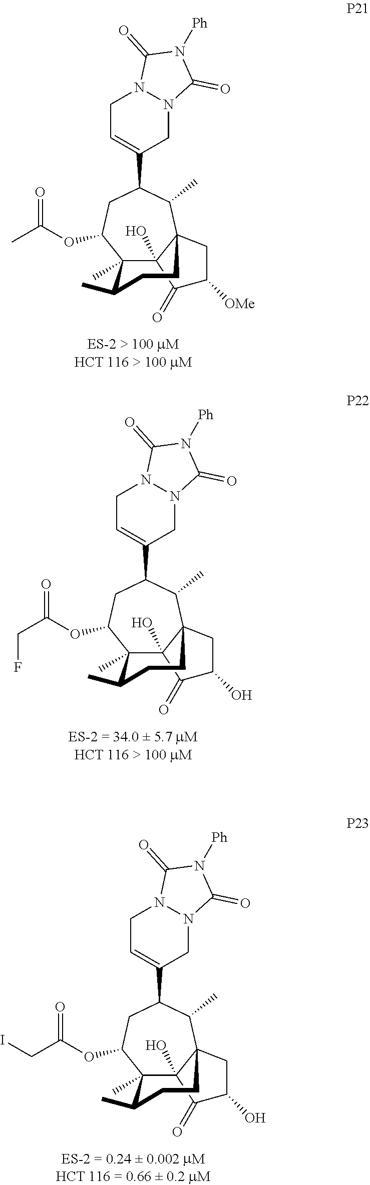

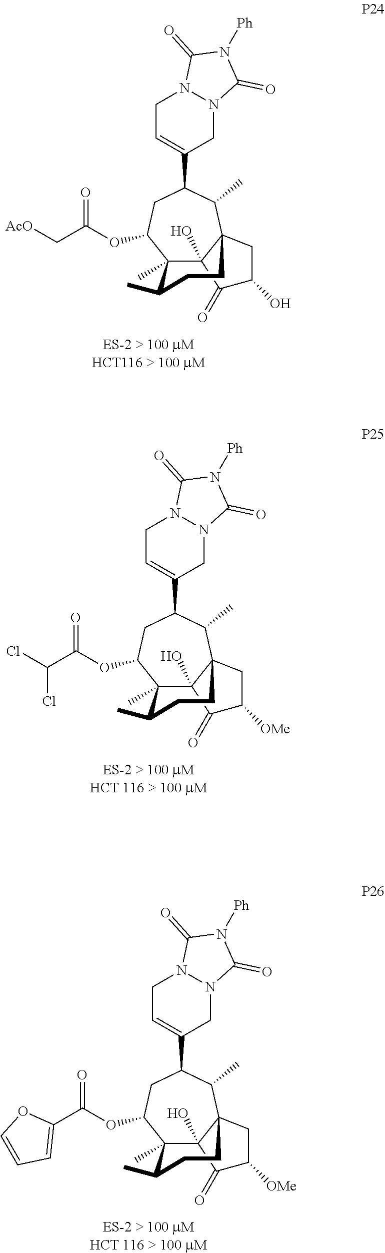

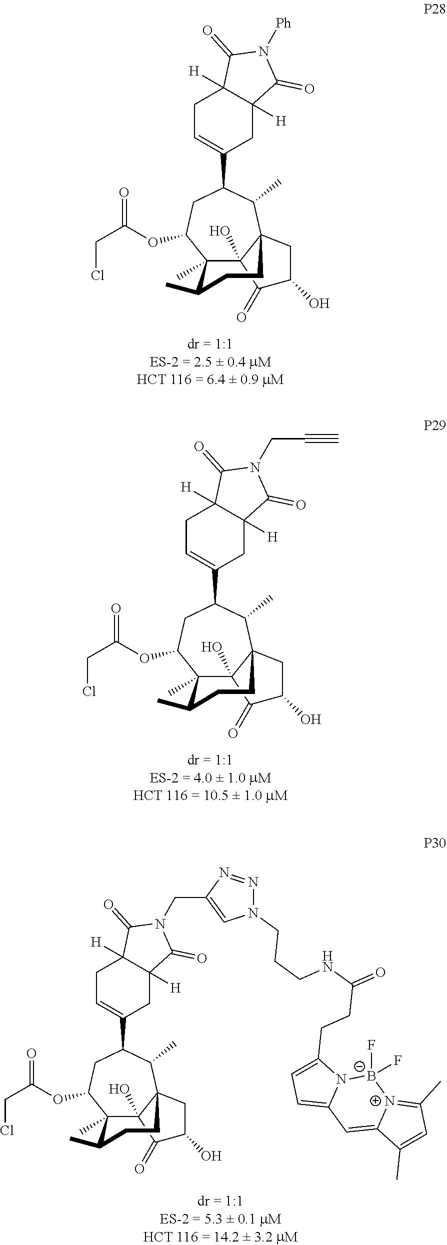

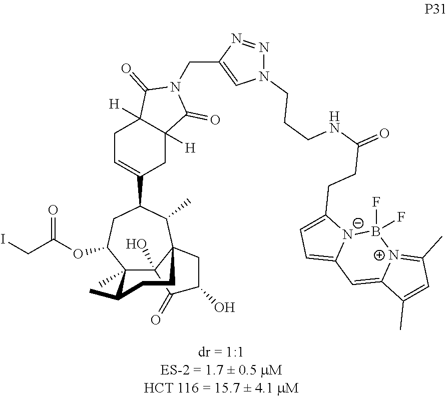

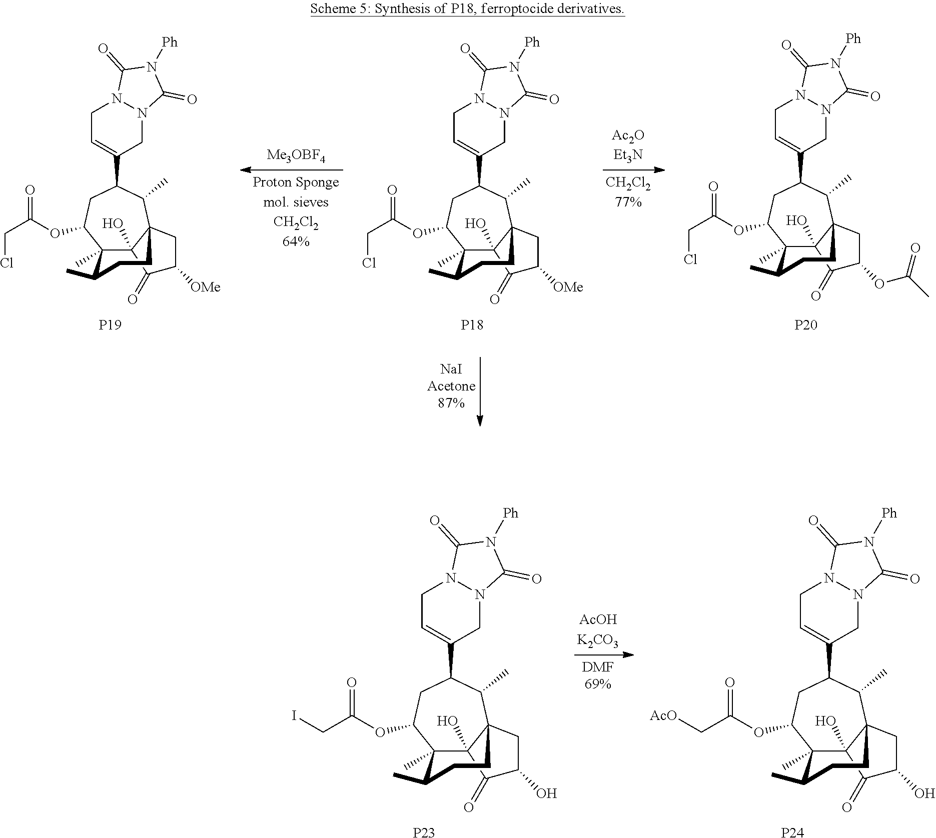

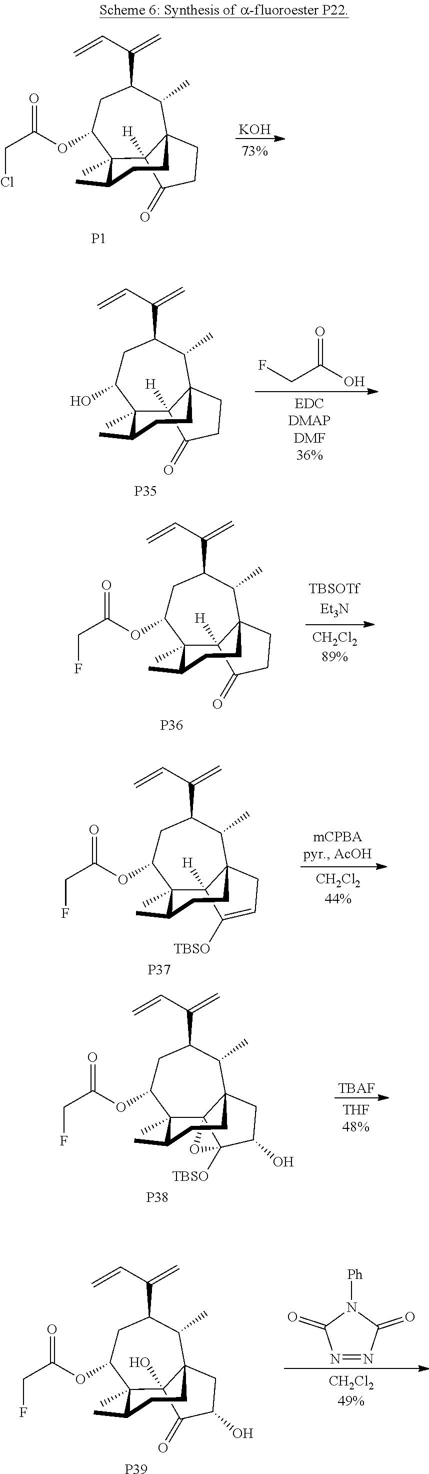

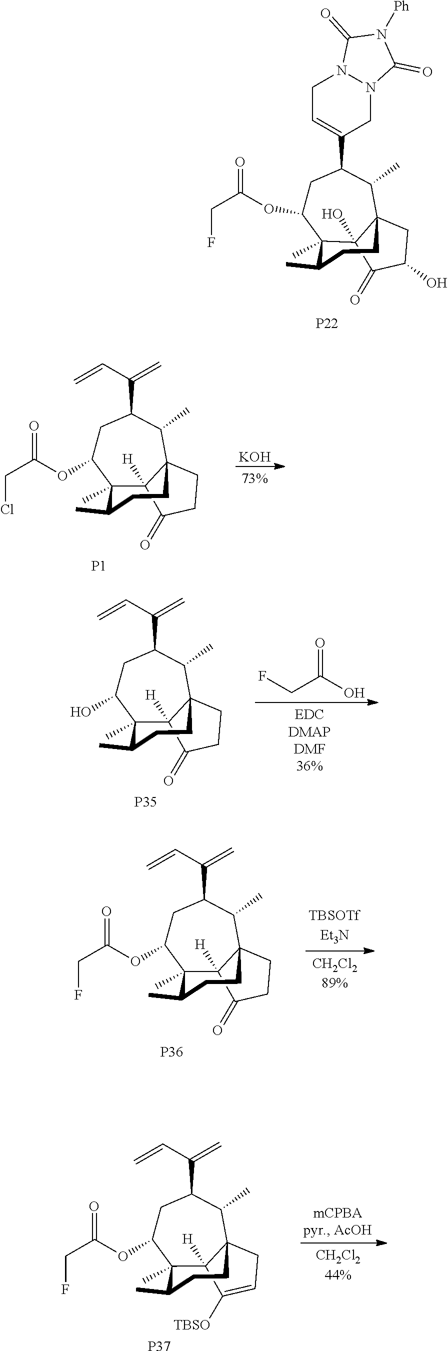

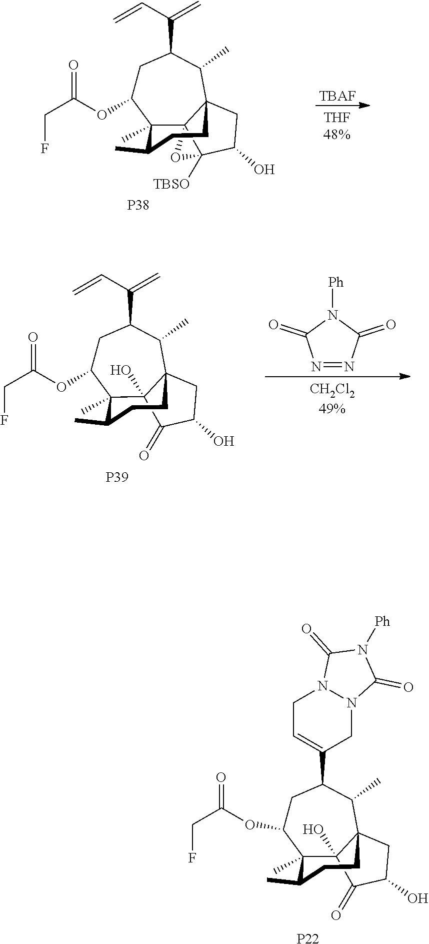

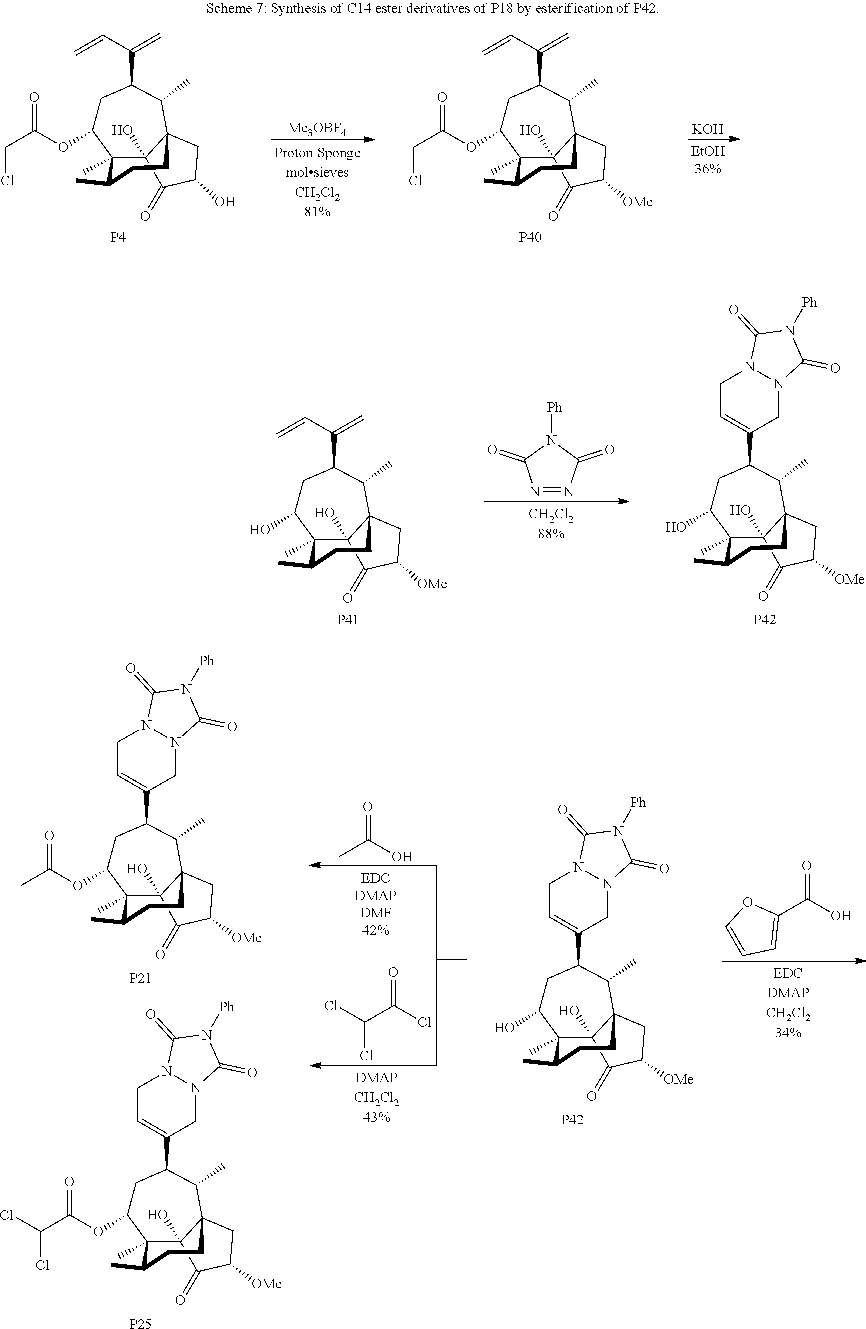

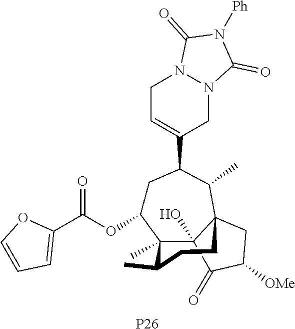

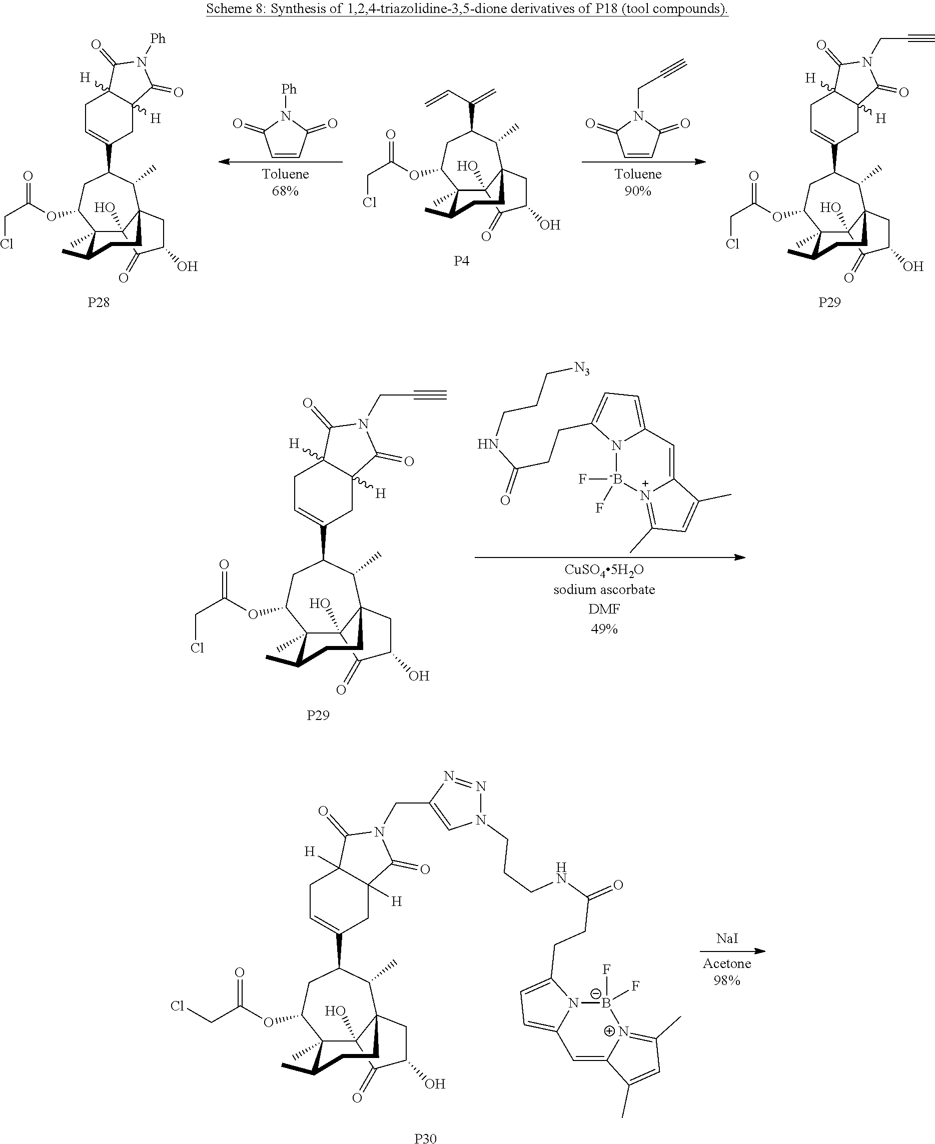

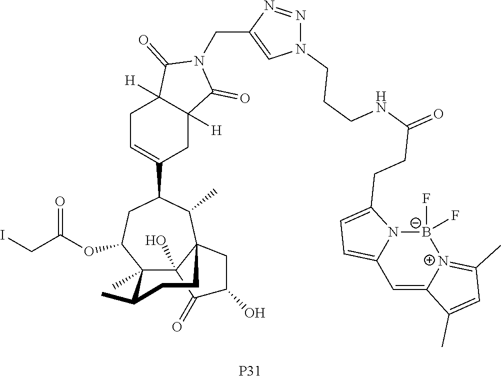

### Example 34. Methods and Characterization

All chemical reagents were purchased from commercial sources and used without further purification. Pleuromutilin was purchased from Waterstone Technology (95% purity) and Bosche Scientific LLC (90% purity) and was used as received. Anhydrous dichloromethane, tetrahydrofuran, methanol, N,N-dimethylformamide, and acetonitrile used in this study were dried by percolation through columns packed with activated alumina under positive pressure of nitrogen. Reactions were monitored by thin layer chromatography using phosphomolybdic acid with cerium sulfate and heat or KMnO₄ and heat as developing agents. Flash chromatography was performed using silica gel (230-400 mesh).

NMR spectra were recorded on Varian Unity spectrometers at 500 MHz for ¹H NMR and 125 MHz for ¹³C NMR. Spectra were obtained in the following solvents (reference peaks included for ¹H & ¹³C NMR): CDCl₃ (¹H NMR: 7.26 ppm; ¹³C NMR: 77.23 ppm), C₆D₆ (¹H NMR: 7.16 ppm; ¹³C NMR: 128.06 ppm) and DMF-d₇ (¹H NMR: 8.03 ppm; ¹³C NMR: 163.15 ppm). NMR experiments were performed at room temperature unless otherwise indicated. Chemical shift values for all ¹H NMR and ¹³C NMR spectra are reported in parts per million (ppm). ¹H NMR multiplicities are reported as: s=singlet, d=doublet, t=triplet, q=quartet, p=pentet, sept=septet, m=multiplet, br=broad.

High resolution mass spectra (HRMS) were acquired using Waters Q-TOF Ultima ESI and Agilent 6230 ESI TOF LC/MS spectrometers. LCMS spectra were collected using an Agilent 6230 ESI TOF LC/MS spectrometers (10 μL injection) with Agilent eclipse plus C18 columns (1.8 μm, 2.1×50 mm) with a gradient of 2.5-80% acetonitrile in water with 0.1% formic acid (0 min 2.5%, 1 min 2.5%, 7 min 80%, 8 min 80%, 9 min 2.5%, 10 min 2.5%).

Pleuromutilin Derived Compounds Synthesis and Characterization.

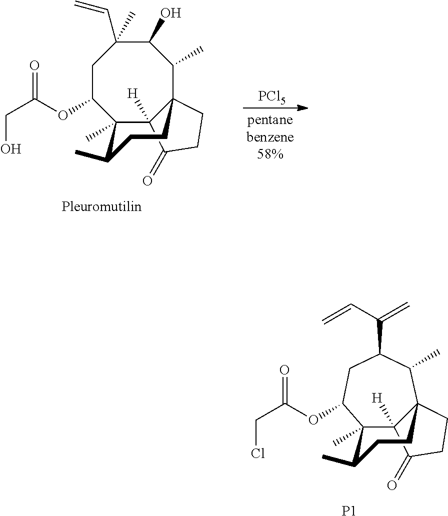

Procedure: To a stirred suspension of pleuromutilin (6.0 g, 16 mmol) in benzene (150 mL) and pentane (150 mL) at 0° C. was added phosphorous pentachloride (10.0 g, 48 mmol). The reaction mixture was stirred at 0° C. for 1 hour then poured onto ice. The crude mixture was extracted with ethyl acetate, washed with sodium bicarbonate and brine, dried with magnesium sulfate, passed through a plug of silica (1:1 ethyl acetate:hexanes elution), and evaporated. The partially purified product was suspended in ethanol and sonicated. Filtration provided pure diene P1 (3.5 g, 58% yield). Crystals suitable for X-ray crystallography were grown by slow evaporation in ethyl acetate or by vapor diffusion (inner vial: 1:1 dichloromethane:ethyl acetate, outer vial: hexanes).

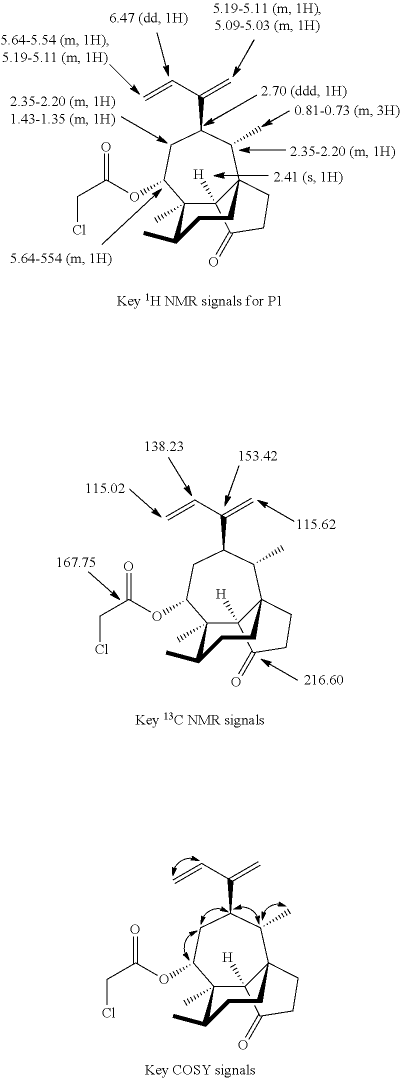

¹H NMR (d7-DMF, 500 MHz, 80° C.): δ 6.47 (dd, J=17.6, 11.0 Hz, 1H), 5.64-5.54 (m, 2H), 5.19-5.11 (m, 2H), 5.09-5.03 (m, 1H), 4.38-4.25 (m, 2H), 2.70 (ddd, J=11.6, 3.8 Hz, 1H), 2.41 (s, 1H), 2.35-2.20 (m, 3H), 2.20-2.09 (m, 1H), 1.81 (ddd, J=12.7, 9.7 Hz, 1H), 1.76-1.66 (m, 1H), 1.66-1.47 (m, 4H), 1.43 (s, 3H), 1.43-1.35 (m, 1H), 1.21 (ddd, J=17.5, 12.8, 4.5 Hz, 1H), 0.81-0.73 (m, 6H). ¹³C NMR (d7-DMF, 125 MHz, 80° C.): δ 216.59, 167.75, 153.15, 138.55, 115.63, 115.02, 72.78, 60.33, 46.41, 46.26, 42.52, 42.04, 41.70, 36.70, 36.18, 35.65, 30.79, 28.69, 26.50, 16.53, 15.91, 14.82. HRMS(ESI): m/z calc. for C₂₂H₃₁ClO₃Na [M+Na]⁺: 401.1854, found: 401.1850.

### Example 35. Crystallography

Experimental Protocol: Intensity data were collected on a Siemens platform diffractometer equipped with an APEXII CCD detector. A normal focus sealed tube Mo source (λ=0.71073 Å) coupled with a graphite monochromator provided the incident beam. The sample was mounted on a 0.3 mm loop with the minimal amount of Paratone-N oil. Data was collected as a series of φ and/or ω scans. Data was collected at 183 K using a cold stream of N_{2(g)}. The data collection was carried out with the APEX2 software. Cell refinement and integration of intensity data was performed with SAINT then corrected for absorption by integration using SHELXTL/XPREP before using SADABS to sort, merge, and scale the combined data. The structure was phased with direct methods using SHELXS and refined with the full-matrix least-squares program SHELXL. CCDC: 1851845.

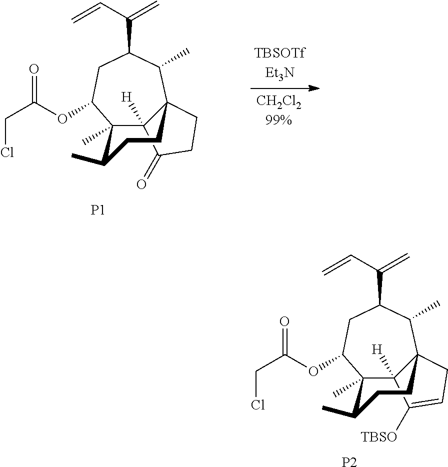

Procedure: To a solution of diene P1 (800 mg, 2.11 mmol) and triethylamine (1.77 mL, 12.7 mmol) in dichloromethane (35 mL) at 0° C. was added tert-butyldimethylsilyl trifluoromethanesulfonate (1.46 mL, 6.33 mmol). The reaction was stirred for 2 hours while warming to room temperature before being quenched by addition of water. The biphasic mixture was extracted with dichloromethane, washed with brine, dried with magnesium sulfate, and evaporated. Purification by flash chromatography (1:9 ethyl acetate:hexanes) provided silyl enol ether P2 (1.04 g, 99%) as a colorless oil.

Note: The reaction to form P2 exclusively provides the tri-substituted silyl enol ether when fresh tert-butyldimethylsilyl trifluoromethanesulfonate is used. It was found that aged bottles of tert-butyldimethylsilyl trifluoromethanesulfonate may produce a mixture of the tri- and tetra-substituted silyl enol ether isomers. Additionally, isomerization of the tri-substituted enol ether will occur upon prolonged exposure to silica gel. However, it was found that the tetra-substituted silyl enol ether isomerizes cleanly to the tri-substituted silyl enol ether after stirring for 1 hour at −78° C. under the Rubottom oxidation conditions employed in the synthesis of P3; thus, both isomers provide epoxide P3 after warming the reaction to room temperature.

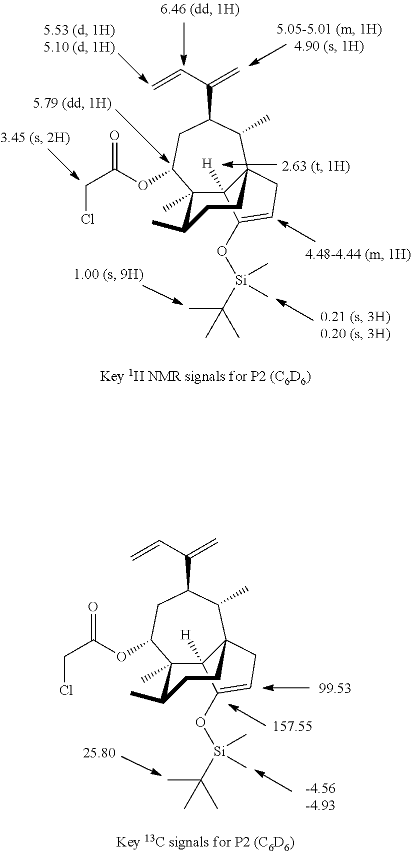

¹H-NMR (C₆D₆, 500 MHz, 60° C.): δ 6.46 (dd, J=17.6, 11.1 Hz, 1H), 5.79 (dd, J=12.2, 2.8 Hz, 1H), 5.53 (d, J=17.6 Hz, 1H), 5.09 (d, J=11.0 Hz, 1H), 5.02 (s, 1H), 4.89 (s, 1H), 4.46 (s, 1H), 3.45 (s, 2H), 2.62 (s, 1H), 2.39 (td, J=11.6, 4.3 Hz, 1H), 2.14 (dq, J=13.2, 6.7 Hz, 1H), 2.09-2.01 (m, 2H), 1.83 (td, J=13.5, 5.6 Hz, 1H), 1.76-1.54 (m, 3H), 1.48 (s, 3H), 1.41 (dd, J=14.0, 3.3 Hz, 1H), 1.32 (d, J=14.5 Hz, 1H), 1.23 (dd, J=13.7, 4.6 Hz, 1H), 1.00 (s, 9H), 0.80 (d, J=6.9 Hz, 3H), 0.73 (d, J=6.8 Hz, 3H), 0.20 (s, 3H), 0.19 (s, 3H).

¹³C-NMR (C₆D₆, 125 MHz, 60° C.): δ 165.90, 157.54, 152.21, 136.90, 114.87, 113.64, 99.58, 72.66, 70.16, 53.60, 48.48, 40.91, 40.77, 36.54, 35.31, 34.88, 31.84, 28.08, 25.80, 18.18, 17.10, 16.24, 15.15, −4.57, −4.91. HRMS(ESI): m/z calc. for C₂₈H₄₆O₃SiCl [M+H]⁺: 493.2899, found: 493.2904.

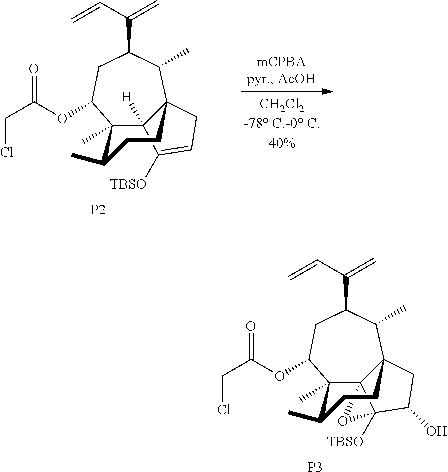

Procedure: To a solution of m-chloroperoxybenzoic acid (1.4 g, 6 mmol), pyridine (886 μL, 11 mmol), and glacial acetic acid (2.6 mL, 46 mmol) in dichloromethane (18 mL) at −78° C. was added a solution of silyl enol ether P2 (1.0 g, 2 mmol) in dichloromethane (2 mL). The reaction was stirred at −78° C. for one hour then warmed to 0° C. and stirred an additional 3 hours while allowing the reaction to warm to room temperature. The reaction was quenched by addition saturated sodium sulfite and saturated sodium bicarbonate. The biphasic mixture was then extracted with dichloromethane and the combined organic layers were washed with brine, dried with magnesium sulfate, and evaporated. Purification by flash chromatography (1:5 ethyl acetate:hexanes) provided a single isomer of epoxide P3 (420 mg, 40%) as a white foam.

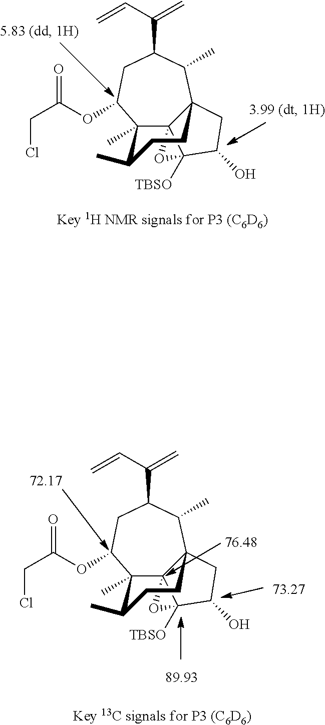

¹H-NMR (C₆D₆, 500 MHz, 60° C.): δ 6.52 (dd, J=17.6, 11.0 Hz, 1H), 5.83 (dd, J=11.9, 2.7 Hz, 1H), 5.56 (d, J=17.5 Hz, 1H), 5.12 (d, J=11.0 Hz, 1H), 5.06 (s, 1H), 5.01 (bs, 1H), 3.99 (dt, J=10.0, 7.6 Hz, 1H), 3.42 (s, 2H), 3.01-2.81 (m, 2H), 2.23 (dq, J=11.5, 6.8 Hz, 1H), 1.74-1.46 (m, 3H), 1.41 (s, 2H), 1.33 (ddd, J=13.7, 4.4, 2.2 Hz, 1H), 1.24-1.11 (m, 2H), 1.03 (s, 9H), 1.01 (d, J=17.2 Hz, 1H), 0.98-0.87 (m, 2H), 0.79 (d, J=6.8 Hz, 3H), 0.58 (d, J=6.7 Hz, 3H), 0.38 (s, 3H), 0.36 (s, 3H).

¹³C-NMR (C₆D₆, 125 MHz, 60° C.): δ 166.25, 152.71, 137.56, 115.21, 113.86, 90.07, 76.62, 73.41, 72.31, 46.26, 44.84, 42.70, 41.23, 40.38, 38.35, 35.69, 34.99, 27.75, 26.53, 18.84, 16.53, 14.52, 14.20, −2.61, −2.85. HRMS(ESI): m/z calc. for C₂₈H₄₅O₅SiClNa [M+Na]⁺: 547.2617, found: 547.2621.

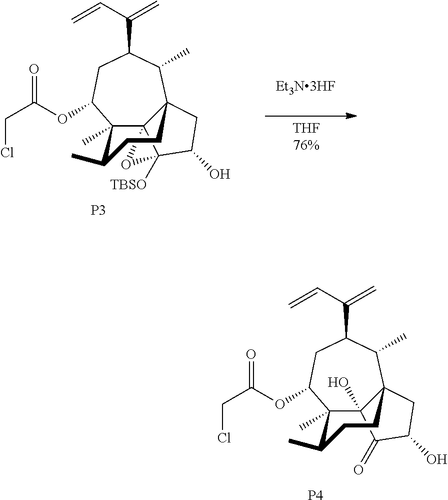

Procedure: A solution of epoxide P3 (878 mg, 1.67 mmol) and triethylamine trihydrofluoride (2.72 mL, 16.7 mmol) in tetrahydrofuran (14 mL) was heated at 60° C. for 3 hours. The reaction was then cooled to room temperature and quenched by addition of a solution of saturated sodium bicarbonate. The biphasic mixture was then extracted with ethyl acetate and the combined organic layers were washed with brine, dried with magnesium sulfate, and evaporated. The crude material was purified by flash chromatography (2:3 ethyl acetate:hexanes) to provide diol P4 (520 mg, 76%) as a white solid.

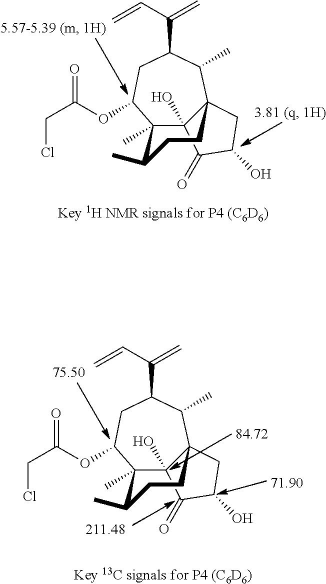

¹H-NMR (C₆D₆, 500 MHz, 60° C.): δ 6.41 (dd, J=17.4, 10.9 Hz, 1H), 5.57-5.39 (m, 2H), 5.06 (d, J=11.0 Hz, 1H), 5.01 (s, 1H), 4.91 (s, 1H), 3.81 (q, J=6.7, 6.2 Hz, 1H), 3.47 (d, J=3.2 Hz, 2H), 3.37-3.27 (m, 1H), 3.14 (d, J=32.6 Hz, 1H), 2.94 (d, J=50.8 Hz, 1H), 2.53-2.39 (m, 1H), 2.14 (ddd, J=12.6, 7.3, 2.2 Hz, 1H), 1.96 (td, J=7.0, 3.1 Hz, 1H), 1.81 (ddd, J=14.8, 7.0, 3.1 Hz, 1H), 1.72-1.58 (m, 2H), 1.49 (d, J=2.8 Hz, 3H), 1.38 (qd, J=13.6, 4.4 Hz, 1H), 1.15-1.08 (m, 1H), 1.02-0.90 (m, 1H), 0.81 (d, J=6.9 Hz, 3H), 0.73 (d, J=6.9 Hz, 3H), 0.69-0.56 (m, 1H).

¹³C-NMR (C₆D₆, 125 MHz, 60° C.): δ 211.72, 166.31, 153.69, 138.20, 115.05, 113.99, 85.04, 75.92, 72.23, 46.01, 44.75, 41.36, 40.22, 39.17, 38.13, 37.23, 36.01, 27.77, 17.01, 16.33, 16.24, 15.73.

HRMS(ESI): m/z calc. for C₂₂H₃₁O₅ClNa [M+Na]⁺: 433.1752, found: 433.1756.

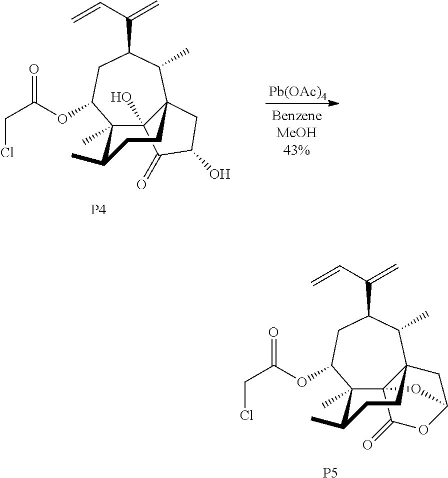

Procedure: To a flask containing diol P4 (100 mg, 0.24 mmol) in benzene (10 mL) and methanol (5 mL) at 0° C. was added lead tetraacetate (129 mg, 0.29 mmol). The reaction was stirred at 0° C. for 90 minutes then quenched by addition of a saturated solution of sodium bicarbonate and extracted with ethyl acetate. The combined organic layers were washed with brine, dried with magnesium sulfate, and evaporated. Purification by flash chromatography (1:4 ethyl acetate:hexanes) afforded a single isomer of lactone P5 (42 mg, 43%) as a white solid.

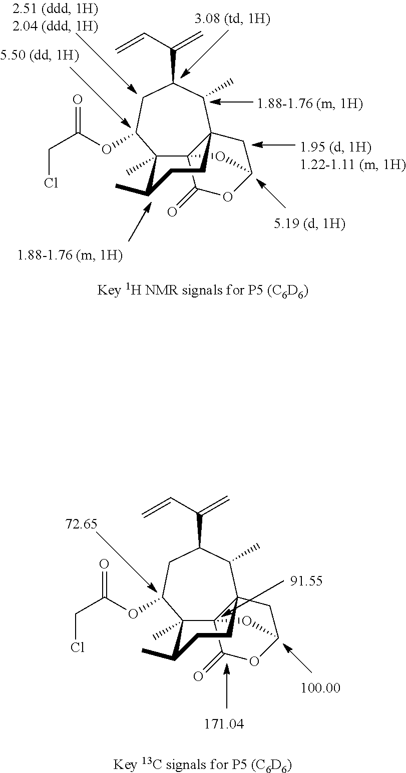

¹H-NMR (C₆D₆, 500 MHz, 60° C.): δ 6.23 (dd, J=17.5, 11.0 Hz, 1H), 5.55 (d, J=17.5 Hz, 1H), 5.50 (dd, J=6.9, 3.8 Hz, 1H), 5.19 (d, J=3.0 Hz, 1H), 5.01 (d, J=11.1 Hz, 1H), 4.91 (s, 1H), 4.77 (s, 1H), 3.67 (d, J=1.8 Hz, 2H), 3.08 (td, J=12.7, 4.3 Hz, 1H), 2.51 (ddd, J=15.7, 6.7, 4.4 Hz, 1H), 2.04 (ddd, J=15.9, 9.5, 3.8 Hz, 1H), 1.95 (d, J=13.2 Hz, 1H), 1.88-1.76 (m, 2H), 1.55-1.37 (m, 2H), 1.24 (s, 3H), 1.22-1.11 (m, 3H), 0.78 (d, J=7.0 Hz, 3H), 0.72 (d, J=7.1 Hz, 3H).

¹³C-NMR (C₆D₆, 125 MHz, 60° C.): δ 171.17, 166.72, 153.09, 138.19, 114.79, 114.18, 100.14, 91.69, 72.78, 45.97, 45.68, 42.30, 41.22, 41.03, 40.20, 39.60, 38.83, 37.43, 28.68, 28.60, 19.69, 16.13.

HRMS(ESI): m/z calc. for C₂H₉O₅ClNa [M+Na]⁺: 431.1596, found: 431.1597.

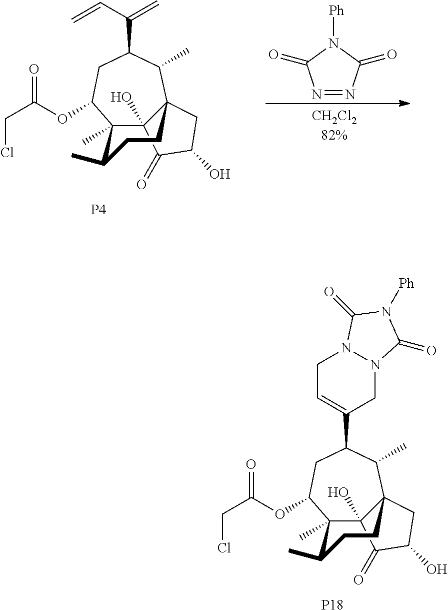

Procedure: To a solution of diene P4 (100 mg, 0.24 mmol) in dichloromethane (2.4 mL) was added 4-Phenyl-1,2,4-triazole-3,5-dione (63 mg, 0.36 mmol) and the reaction was stirred for 3 hours at room temperature. Evaporation and purification by flash chromatography (2:1 ethyl acetate:hexanes) gave P18 (101 mg, 82%) as a white solid.

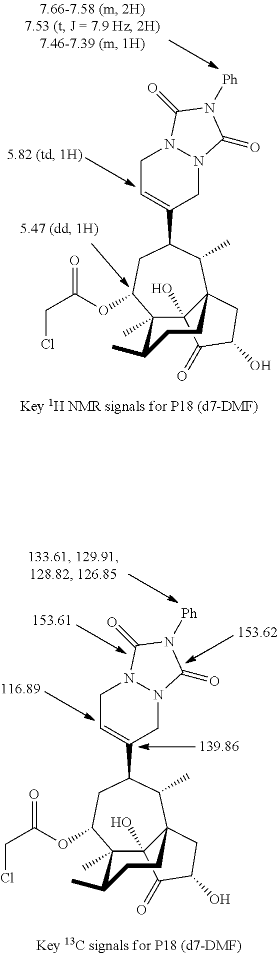

¹H-NMR (d7-DMF, 500 MHz, 80° C.): δ 7.63-7.55 (m, 2H), 7.53 (t, J=7.9 Hz, 2H), 7.46-7.39 (m, 1H), 5.82 (td, J=3.2, 1.6 Hz, 1H), 5.47 (dd, J=10.5, 3.1 Hz, 1H), 5.16 (s, 1H), 5.07 (s, 1H), 4.41-4.34 (m, 1H), 4.36 (d, J=2.1 Hz, 2H), 4.23-4.17 (m, 1H), 4.16 (h, J=2.2 Hz, 2H), 3.99 (t, J=7.8 Hz, 1H), 3.30-3.21 (m, 1H), 2.76-2.69 (m, 1H), 2.17 (ddd, J=16.9, 12.2, 7.0 Hz, 2H), 1.88 (dd, J=12.1, 8.4 Hz, 1H), 1.80-1.62 (m, 4H), 1.50 (s, 3H), 1.46-1.38 (m, 1H), 1.11 (ddd, J=14.8, 12.2, 5.5 Hz, 1H), 0.93 (d, J=6.9 Hz, 3H), 0.87 (d, J=6.1 Hz, 3H). ¹³C-NMR (d7-DMF, 125 MHz, 80° C.): δ 211.35, 167.98, 153.62 (2 C overlapping), 139.86, 133.61, 129.91, 128.82, 126.85, 116.89, 84.20, 74.90, 72.07, 46.37, 45.21, 44.35, 44.27, 43.35, 42.51, 39.34, 38.39, 38.27, 37.31, 36.58, 28.10, 17.15, 16.74, 14.68.

Crystallography. Experimental Protocol: Intensity data were collected on a Bruker D8 Venture kappa diffractometer equipped with a Photon 100 CMOS detector. An Iμs microfocus Mo source (λ=0.71073 Å) coupled with a multi-layer mirror monochromator provided the incident beam. The sample was mounted on a 0.3 mm loop with the minimal amount of Paratone-N oil. Data was collected as a series of φ and/or ω scans. Data was collected at 100 K using a cold stream of N_{2(g)}. The collection, cell refinement, and integration of intensity data was carried out with the APEX3 software. A semi-empirical absorption correction was performed with SADABS. The structure was phased with intrinsic methods using SHELXT and refined with the full-matrix least-squares program SHELXL. CCDC: 1849494.

HRMS(ESI): m/z calc. for C₃₀H₃₇N₃O₇Cl [M+H]⁺: 586.2315, found: 586.2313.

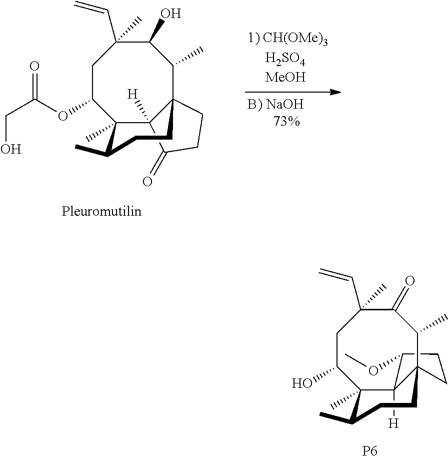

Procedure: Pleuromutilin (1.00 g, 2.6 mmol) was dissolved in trimethyl orthoformate (1.28 mL, 11.7 mmol) and methanol (3.7 ml) and cooled to 0° C. concentrated sulfuric acid (0.283 mL, 5.2 mmol) was then added dropwise with stirring. The reaction was then heated to 30° C., stirred for 8 hours, then cooled to room temperature. A solution of sodium hydroxide (832 mg, 20.8 mmol) in water (0.87 mL) was then added and the reaction was heated at 60° C. for 2 hours. The crude reaction mixture was then cooled, diluted with water, and extracted with ethyl acetate. The combined organic layers were washed with brine, dried with magnesium sulfate, and evaporated. Purification by flash chromatography (1:5 ethyl acetate:hexanes) provided alcohol P6 (637 mg, 73%) as white solid.

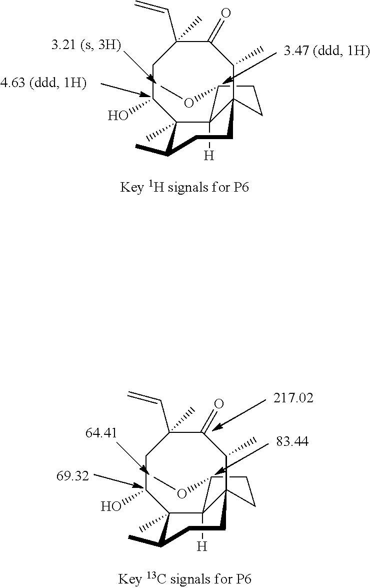

¹H-NMR (CDCl₃, 500 MHz): δ 6.00 (dd, J=17.6, 10.8 Hz, 1H), 5.27 (d, J=10.5 Hz, 1H), 5.24 (d, J=17.6 Hz, 1H), 4.63 (dd, J=9.2, 5.9 Hz, 1H), 3.47 (ddd, J=11.3, 8.1, 5.5 Hz, 1H), 3.21 (s, 3H), 2.91 (q, J=6.5 Hz, 1H), 2.42 (dd, J=15.3, 9.3 Hz, 1H), 2.19 (ddd, J=13.6, 10.2, 3.5 Hz, 1H), 2.04-1.91 (m, 2H), 1.82 (d, J=15.3 Hz, 1H), 1.71 (d, J=11.3 Hz, 1H), 1.55-1.49 (m, 2H), 1.46 (dtd, J=14.7, 4.1, 2.7 Hz, 1H), 1.34 (dtd, J=18.6, 6.9, 3.9 Hz, 1H), 1.23 (tdd, J=12.3, 5.4, 3.4 Hz, 1H), 1.16 (s, 3H), 1.15 (s, 3H), 1.14-1.09 (m, 1H), 1.07 (d, J=7.0 Hz, 3H), 0.97 (d, J=6.5 Hz, 3H). ¹³C-NMR (CDCl₃, 125 MHz): δ 217.02, 140.77, 117.23, 83.44, 69.32, 64.41, 57.02, 54.66, 47.86, 45.64, 44.98, 44.38, 44.34, 40.75, 30.82, 29.65, 29.05, 26.01, 19.08, 18.13, 15.41. HRMS(ESI): m/z calc. for C₂₁H₃₄O₃Na [M+Na]⁺: 357.2400, found: 357.2393.

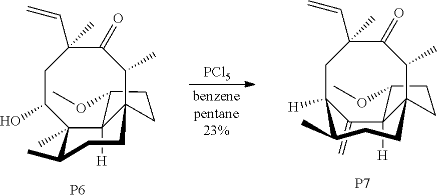

Procedure: To a solution of alcohol P6 (50 mg, 0.15 mmol) in benzene (2.5 mL) and pentane (2.5 mL) at room temperature was added phosphorus pentachloride (34 mg, 0.16 mmol). The reaction was stirred 15 minutes and quenched by addition of water. The biphasic mixture was extracted with ethyl acetate and the combined organic layers were washed with brine, dried with magnesium sulfate, and evaporated. The crude product was purified by flash chromatography (3% ethyl acetate in hexanes) to provide a single isomer of P7 (11 mg, 23%) as a white solid.

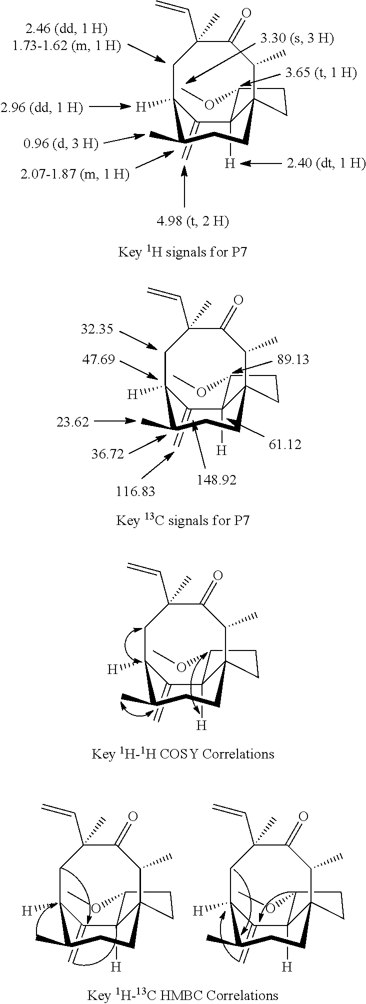

¹H-NMR (CDCl₃, 500 MHz): δ 5.66 (dd, J=17.4, 10.6 Hz, 1H), 5.14-5.09 (m, 1H), 5.09-5.05 (m, 1H), 4.97 (s, 2H), 3.64 (t, J=6.6 Hz, 1H), 3.29 (s, 3H), 3.19 (q, J=7.0 Hz, 1H), 2.95 (dd, J=12.0, 5.5 Hz, 1H), 2.45 (dd, J=15.4, 5.6 Hz, 1H), 2.39 (s, 1H), 2.06-1.86 (m, 4H), 1.83 (dd, J=13.0, 6.7 Hz, 1H), 1.73-1.63 (m, 2H), 1.50 (d, J=12.4 Hz, 1H), 1.26 (s, 5H), 0.95 (d, J=6.8 Hz, 3H), 0.93 (d, J=7.2 Hz, 3H). ¹³C-NMR (CDCl₃, 125 MHz): δ 216.72, 148.92, 143.49, 116.83, 114.89, 89.13, 61.12, 56.45, 56.05, 49.99, 47.69, 44.33, 39.63, 37.40, 36.71, 32.35, 29.15, 28.83, 23.62, 22.77, 16.59. HRMS(ESI): m/z calc. for C₂H₃₃O₂[M+H]⁺; 317.2475, found: 317.2473.

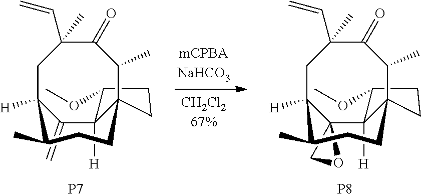

Procedure: Olefin P7 (50 mg, 0.16 mmol) was dissolved in dichloromethane (1.6 m) then sodium bicarbonate (27 mg, 0.32 mmol) and m-chloroperoxy benzoic acid (44 mg, 0.19 mmol) were added. The reaction was stirred at room temperature for 5 hours then quenched by addition of a saturated solution of sodium sulfite and extracted with dichloromethane. The combined organic layers were washed with brine, dried with magnesium sulfate, and evaporated. Purification by flash chromatography (1:4 ethyl acetate:hexanes) provided a single isomer of epoxide P8 (36 mg, 67%) as a white solid.

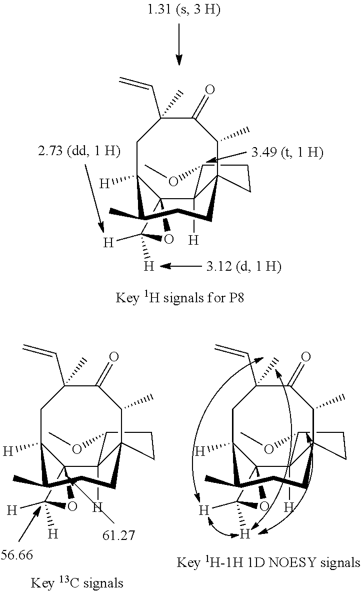

¹H-NMR (CDCl₃, 500 MHz): δ 5.72 (dd, J=17.4, 10.6 Hz, 1H), 5.15 (d, J=17.6 Hz, 1H), 5.14 (d, J=10.4 Hz, 1H), 3.49 (t, J=7.6 Hz, 1H), 3.29 (q, J=7.1 Hz, 1H), 3.20 (s, 3H), 3.12 (d, J=4.6 Hz, 1H), 2.73 (dd, J=4.7, 1.1 Hz, 1H), 2.43 (dd, J=15.7, 5.1 Hz, 1H), 2.38-2.28 (m, 1H), 2.21 (d, J=7.1 Hz, 1H), 2.04-1.87 (m, 3H), 1.84 (dd, J=13.1, 6.9 Hz, 1H), 1.73 (dd, J=15.7, 12.4 Hz, 1H), 1.62-1.50 (m, 3H), 1.41-1.33 (m, 1H), 1.31 (s, 3H), 1.27 (dd, J=11.9, 6.7 Hz, 1H), 0.98 (d, J=7.0 Hz, 3H), 0.96 (dd, J=7.0, 1.0 Hz, 3H). ¹³C-NMR (CDCl₃, 125 MHz): δ 215.86, 143.48, 115.09, 85.89, 61.27, 59.65, 56.67, 56.66, 55.71, 49.95, 49.03, 44.17, 39.38, 37.60, 35.43, 31.47, 28.49, 28.43, 23.71, 23.51, 16.38. HRMS(ESI): m/z calc. for C₂₁H₃₂O₃Na [M+Na]⁺: 355.2244, found: 355.2249.

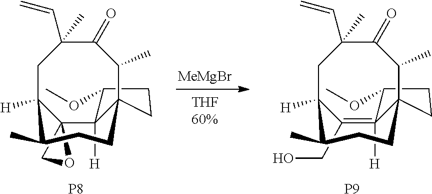

Procedure: Epoxide P8 (12 mg, 0.036 mmol) was dissolved in tetrahydrofuran (0.36 mL) and cooled to 0° C. A 3.0 M solution of methyl magnesium bromide in diethyl ether (36 μL, 0.108 mmol) was then added and the reaction was stirred from 0° C. to room temperature over 4 hours. The reaction was quenched by addition of a saturated solution of ammonium chloride and extracted with ethyl acetate. The combined organic layers were washed with brine, dried with magnesium sulfate and evaporated. Purification by flash chromatography (1:3 ethyl acetate:hexanes) afforded allylic alcohol P9 (7 mg, 60%) as a white solid.

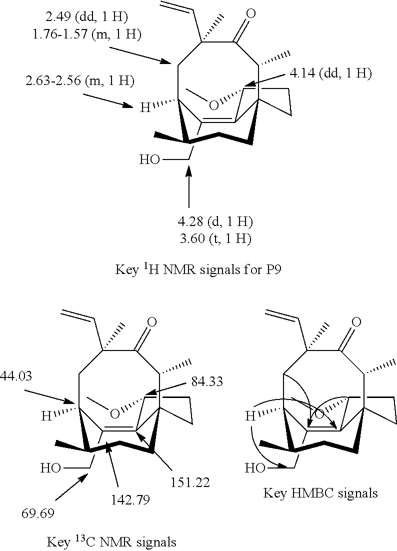

¹H-NMR (CDCl₃, 500 MHz): δ 5.59 (dd, J=17.5, 10.6 Hz, 1H), 5.07 (d, J=17.3 Hz, 1H), 5.06 (d, J=10.7 Hz, 1H), 4.28 (d, J=10.9 Hz, 1H), 4.14 (dd, J=5.8, 3.2 Hz, 1H), 3.78 (q, J=7.0 Hz, 1H), 3.60 (t, J=10.9 Hz, 1H), 3.30 (s, 3H), 2.70 (d, J=11.7 Hz, 1H), 2.63-2.56 (m, 1H), 2.49 (dd, J=15.4, 5.9 Hz, 1H), 2.20-2.06 (m, 1H), 2.04-1.93 (m, 2H), 1.80 (ddt, J=12.4, 10.7, 6.1 Hz, 1H), 1.76-1.57 (m, 3H), 1.53 (td, J=13.4, 4.1 Hz, 1H), 1.39 (ddd, J=13.1, 10.8, 6.5 Hz, 1H), 1.00 (s, 3H), 0.98 (d, J=7.0 Hz, 3H), 0.97 (d, J=6.9 Hz, 3H). ¹³C-NMR (CDCl₃, 125 MHz): δ 214.66, 151.46, 143.03, 142.64, 114.79, 84.57, 69.93, 55.97, 55.61, 47.36, 44.27, 41.44, 38.39, 36.79, 35.56, 33.94, 29.48, 27.74, 22.88, 22.12, 14.68. HRMS(ESI): m/z calc. for C₂₁H₃₂O₃Na [M+Na]⁺: 355.2244, found: 355.2248.

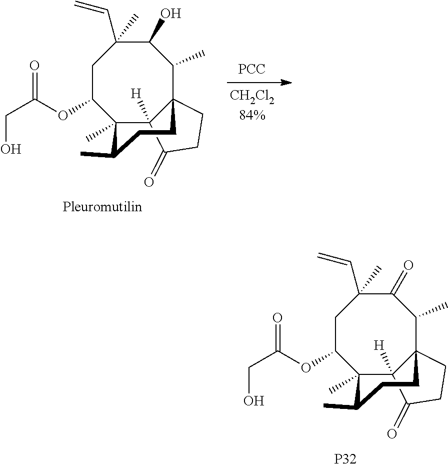

Procedure: Pyridinium chlorochromate (1.71 g, 7.92 mmol) was added to a stirred solution of pleuromutilin (2.00 g, 5.28 mmol) in dichloromethane (80 mL) at room temperature. The reaction was stirred 15 hours, diluted with ether, and passed through a short column of silica (ethyl acetate elution). The crude reaction mixture was evaporated, taken up in a minimal amount of dichloromethane, and hexanes were added to precipitate reaction impurities. Filtration and evaporation provided pleuromutilone P32 (1.67 g, 84%) as a white solid.

¹H-NMR (CDCl₃, 500 MHz): δ 6.64 (dd, J=17.6, 10.8 Hz, 1H), 5.99 (d, J=8.9 Hz, 1H), 5.32 (d, J=10.7 Hz, 1H), 5.03 (d, J=17.6 Hz, 1H), 4.10 (q, J=17.2 Hz, 2H), 3.25 (q, J=6.5 Hz, 1H), 2.61 (s, 1H), 2.28-2.21 (m, 1H), 2.19-2.08 (m, 2H), 2.03 (dd, J=15.6, 9.0 Hz, 1H), 1.66-1.57 (m, 4H), 1.48 (d, J=15.6 Hz, 1H), 1.43 (s, 3H), 1.46-1.33 (m, 2H), 1.14 (d, J=13.4 Hz, 1H), 1.12 (s, 3H), 1.06 (d, J=6.5 Hz, 3H), 0.72 (d, J=6.1 Hz, 3H). ¹³C-NMR (CDCl₃, 125 MHz): δ 216.68, 214.43, 172.57, 140.21, 118.63, 70.76, 61.53, 59.12, 53.62, 45.74, 45.45, 43.82, 42.20, 37.23, 34.82, 29.98, 26.96, 24.96, 24.86, 17.03, 15.09, 14.04. HRMS(ESI): m/z calc. for C₂₀H₂₉O₂[M-C₂H₃O₃]⁺: 301.2162, found: 301.2171. (loss of ester).

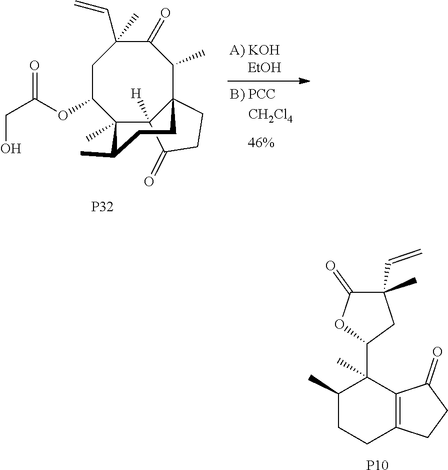

Procedure: Pleuromutilone P32 (613 mg, 1.63 mmol) was added to a solution of 10% potassium hydroxide in ethanol (61 mL) and stirred at reflux for 14 hours. The reaction mixture was then cooled, poured onto ice, acidified with concentrated hydrochloric acid, and extracted with dichloromethane. The combined organic layers were neutralized with saturated sodium bicarbonate, washed with brine, and dried with magnesium sulfate. The crude mixture was then evaporated and taken up in 30 mL of dichloromethane and pyridinium chlorochromate (2.00 g, 9.28 mmol) was added. The reaction was stirred at room temperature 32 hours and passed through a plug of silica (ethyl acetate elution). The crude solution was then adsorbed onto silica by evaporation and purified by flash chromatography using 3:2 ethyl acetate:hexanes to provide lactone P10 (217 mg, 46%).

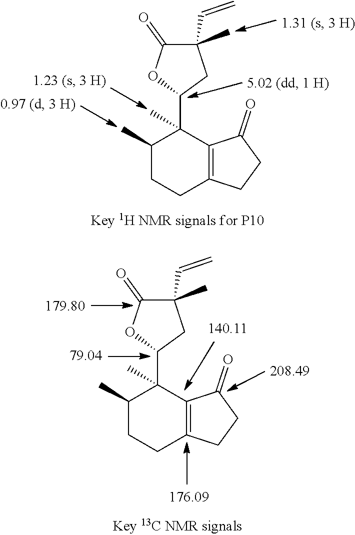 [SMILES: C=C[C@]1(C)C[C@H]([C@@]2(C)C3=C(CCC3=O)CC[C@H]2C)OC1=O ; C=C[C@]1(C)C[C@H]([C@@]2(C)C3=C(CCC3=O)CC[C@H]2C)OC1=O]

¹H-NMR (CDCl₃, 500 MHz): δ 6.01 (dd, J=17.4, 10.7 Hz, 1H), 5.18 (d, J=7.3 Hz, 1H), 5.15 (s, 1H), 5.02 (dd, J=11.2, 5.9 Hz, 1H), 2.62-2.57 (m, 1H), 2.43-2.25 (m, 6H), 1.79 (dd, J=13.0, 6.0 Hz, 2H), 1.68 (tt, J=14.8, 8.2 Hz, 2H), 1.31 (s, 3H), 1.23 (s, 3H), 0.97 (d, J=6.7 Hz, 3H). ¹³C-NMR (CDCl₃, 125 MHz): δ 208.49, 179.80, 176.09, 140.11, 139.93, 114.90, 79.04, 46.48, 40.47, 38.08, 36.30, 35.29, 29.51, 27.43, 26.89, 22.94, 19.06, 16.88. HRMS(ESI): m/z calc. for C₁₈H₂₅O₃[M+H]⁺: 289.1798, found: 289.1813.

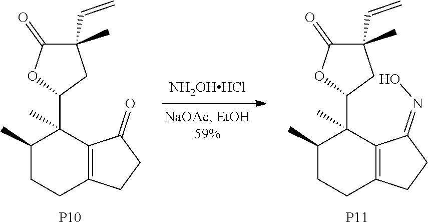

Procedure: Lactone P10 (125 mg, 0.443 mmol) was taken up in ethanol (4.43 mL) then sodium acetate (291 mg, 3.54 mmol) and hydroxylamine hydrochloride (246 mg, 3.54 mmol) were added. The reaction was stirred at reflux for 22 hours then cooled and poured into water. The crude mixture was extracted with dichloromethane, washed with brine, dried with magnesium sulfate, and evaporated. Purification by flash chromatography (2:3 ethyl acetate:hexanes) provided a single diastereomer of oxime P11 (79 mg, 59%) as a white foam.

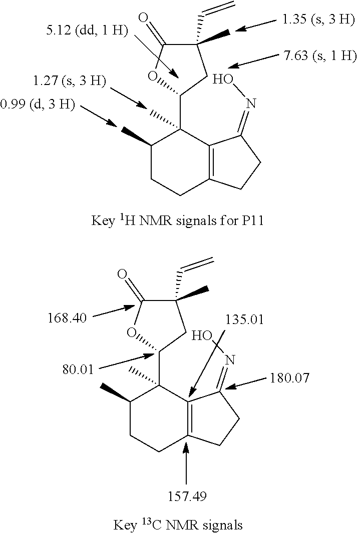 [SMILES: C=C[C@]1(C)C[C@H]([C@@]2(C)C3=C(CC/C3=N/O)CC[C@H]2C)OC1=O ; C=C[C@]1(C)C[C@H]([C@@]2(C)C3=C(CC/C3=N/O)CC[C@H]2C)OC1=O]

¹H-NMR (CDCl₃, 500 MHz): δ 7.63 (s, 1H), 6.04 (dd, J=17.5, 10.6 Hz, 1H), 5.19 (d, J=6.2 Hz, 1H), 5.16 (s, 1H), 5.12 (dd, J=10.9, 6.0 Hz, 1H), 2.72 (ddd, J=18.5, 7.7, 3.5 Hz, 1H), 2.66-2.60 (m, 1H), 2.60-2.53 (m, 1H), 2.41-2.34 (m, 2H), 2.34-2.22 (m, 1H), 2.17-2.09 (m, 1H), 1.88 (dd, J=12.9, 6.0 Hz, 1H), 1.81-1.73 (m, 2H), 1.69-1.62 (m, 1H), 1.35 (s, 3H), 1.27 (d, J=4.7 Hz, 3H), 0.99 (d, J=7.0 Hz, 3H). ¹³C-NMR (CDCl₃, 125 MHz): δ 180.03, 168.36, 157.45, 140.20, 134.96, 114.83, 79.96, 46.72, 41.05, 38.18, 36.73, 32.34, 27.25, 25.93, 24.97, 23.06, 20.74, 17.24. HRMS(ESI): m/z calc. for C₁₈H₂₆NO₃ [M+H]⁺: 304.1907, found: 304.1905.

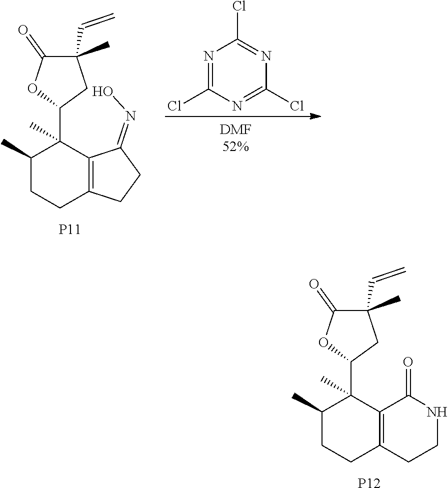

Procedure: Cyanuric chloride (44 mg, 0.24 mmol) was added to 0.3 mL of N,N-dimethylformamide and stirred for 30 minutes until the solution turned yellow. A solution of oxime P11 (36 mg, 0.12 mmol) in N,N-dimethylformamide (3 mL) was then added and the reaction was stirred for 24 hours. The reaction was quenched with water and extracted with dichloromethane. The combined organic layers were then washed with brine, dried with magnesium sulfate, and evaporated. The crude mixture was then taken up in toluene and adsorbed onto silica by evaporation. Purification by flash chromatography (1:4 ethyl acetate:hexanes) afforded a single isomer of pure lactam P12 (19 mg, 52%) as a white solid.

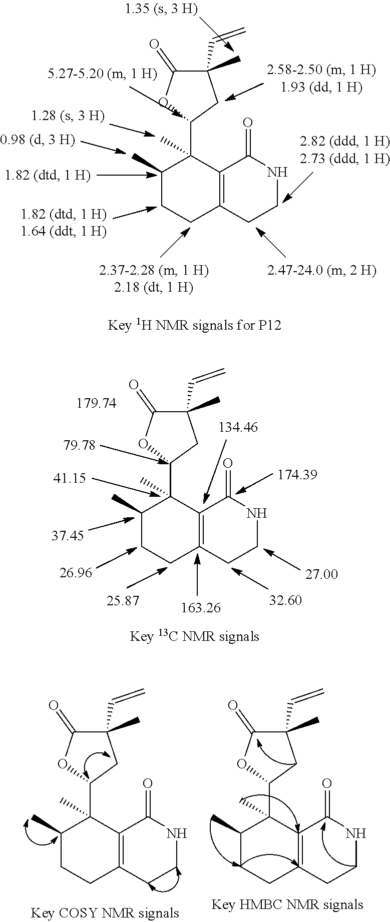

¹H-NMR (CDCl₃, 500 MHz): δ 8.62 (s, 1H), 6.04 (dd, J=17.4, 10.7 Hz, 1H), 5.27-5.20 (m, 1H), 5.19 (d, J=6.3 Hz, 1H), 5.16 (d, J=13.0 Hz, 1H), 2.82 (ddd, J=19.0, 7.2, 3.3 Hz, 1H), 2.73 (ddd, J=19.0, 7.2, 3.6 Hz, 1H), 2.58-2.50 (m, 1H), 2.47-2.40 (m, 2H), 2.37-2.28 (m, 1H), 2.18 (dt, J=19.5, 6.0 Hz, 1H), 1.93 (dd, J=12.8, 6.1 Hz, 1H), 1.82 (dtd, J=14.1, 6.8, 3.4 Hz, 2H), 1.64 (ddt, J=13.6, 7.6, 5.3 Hz, 1H), 1.35 (s, 3H), 1.28 (s, 3H), 0.98 (d, J=6.9 Hz, 3H). ¹³C-NMR (CDCl₃, 125 MHz): δ 179.74, 174.39, 163.26, 140.00, 134.46, 114.92, 79.78, 46.77, 41.15, 37.45, 36.33, 32.60, 27.00, 26.96, 25.87, 23.41, 21.19, 17.05. HRMS(ESI): m/z calc. for C₁₈H₂₆NO₃ [M+H]⁺: 304.1907, found: 304.1907.

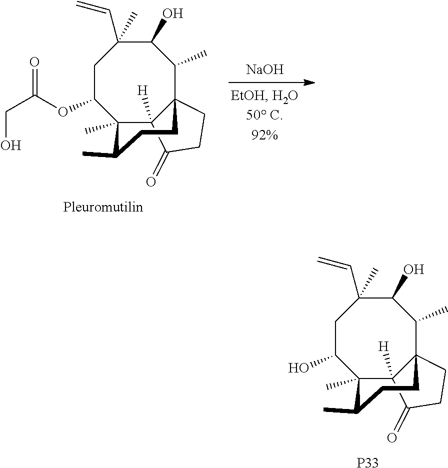

Compound 33 was synthesized as described previously: Briefly, a solution of pleuromutilin (2.4 g, 6.34 mmol, 1 equiv.), 1.26 mL 50% NaOH_{(aq.)}, water (9 mL), and ethanol (14.4 mL) was heated at 50° C. for 3 h under air. Upon cooling, the reaction mixture was diluted with water and concentrated to precipitate the crude product. The solid was filtered, washed with water and hexanes, and then dried under vacuum to provide 1.875 g (92%) of mutilin P33 as an off white solid. ¹H-NMR and ¹³C-NMR spectra matched with the previously published spectral data.

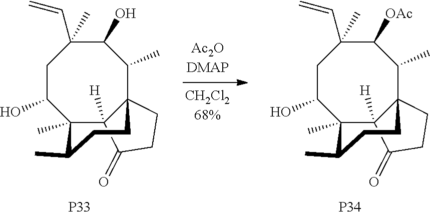

Procedure: To a solution of mutilin P33 (600 mg, 1.6 mmol) and N,N-dimethylamino pyridine (232 mg, 1.9 mmol) in dichloromethane (19 mL) was added acetic anhydride (265 μL, 2.8 mmol). The reaction was stirred 4 hours and then quenched by addition of water and extracted with dichloromethane. The combined organic layers were washed with brine, dried with magnesium sulfate, and evaporated. The crude mixture was purified by flash chromatography (1:4 ethyl acetate:hexanes) to provide acetate P34 (467 mg, 68%) as a white solid.

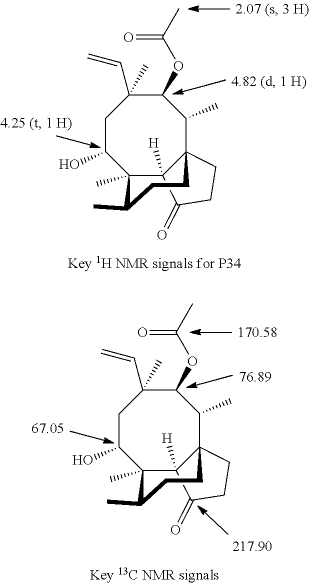

¹H-NMR (CDCl₃, 500 MHz): δ 6.07 (dd, J=17.9, 11.2 Hz, 1H), 5.39 (d, J=17.9 Hz, 1H), 5.23 (d, J=11.2 Hz, 1H), 4.82 (d, J=6.7 Hz, 1H), 4.25 (t, J=6.6 Hz, 1H), 2.31-2.17 (m, 2H), 2.17-2.02 (m, 2H), 2.07 (s, 3H), 1.96-1.80 (m, 2H), 1.70-1.57 (m, 3H), 1.48-1.37 (m, 2H), 1.34 (d, J=1.2 Hz, 3H), 1.34-1.28 (m, 2H), 1.09 (td, J=14.0, 4.3 Hz, 1H), 0.94 (d, J=1.2 Hz, 3H), 0.92 (d, J=7.1 Hz, 3H), 0.75 (d, J=7.1 Hz, 3H). ¹³C-NMR (CDCl₃, 125 MHz): δ 217.98, 170.66, 140.17, 115.80, 76.97, 67.14, 59.41, 45.37, 44.70, 44.35, 42.50, 37.00 (2C), 34.71, 30.54, 29.16, 27.46, 25.29, 20.99, 18.45, 13.68, 12.05. HRMS(ESI): m/z calc. for C₂₂H₃₅O₄[M+H]⁺: 363.2535, found: 363.2529.

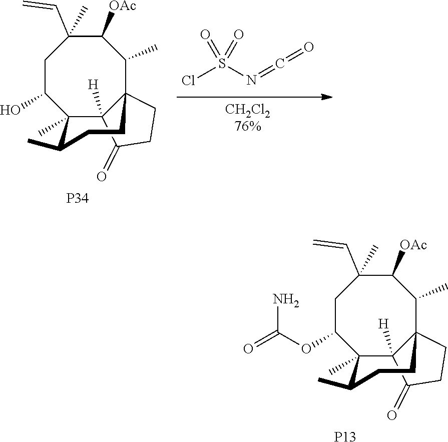

Procedure: To a solution of chlorosulfonyl isocyanate (192 μL, 2.2 mmol) in dichloromethane (8 mL) at 0° C. was added a solution of acetate P34 (400 mg, 1.1 mmol) in dichloromethane (3 mL). The reaction was stirred for 4 hours while warming to room temperature. Upon completion, the reaction mixture was cooled to 0° C. and tetrahydrofuran (3 mL) and water (1.5 mL) were added. The mixture was then heated at 40° C. for 24 hours. The crude reaction was then cooled to room temperature, diluted with water, and extracted with dichloromethane. The combined organic layers were washed with brine, dried with magnesium sulfate, and evaporated. The crude product was purified by flash chromatography (1:2 ethyl acetate:hexanes) to provide carbamate P13 (339 mg, 76%) as a white solid.

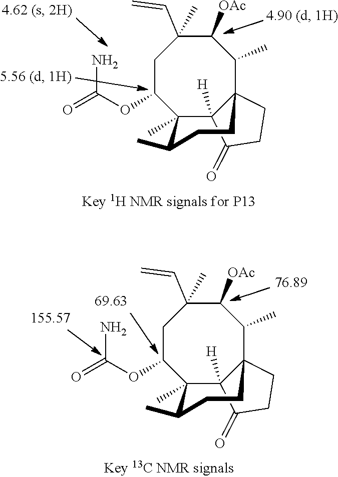

¹H-NMR (CDCl₃, 500 MHz): 6.42 (dd, J=17.5, 11.2 Hz, 1H), 5.56 (d, J=8.2 Hz, 1H), 5.29 (dd, J=11.2, 1.3 Hz, 1H), 5.23 (dd, J=17.5, 1.4 Hz, 1H), 4.90 (d, J=6.7 Hz, 1H), 4.62 (s, 2H), 2.53 (p, J=7.0 Hz, 1H), 2.38-2.29 (m, 1H), 2.25-2.14 (m, 2H), 2.11 (s, 3H), 2.13-2.04 (m, 1H), 1.96-1.86 (m, 1H), 1.76 (dd, J=14.3, 2.9 Hz, 1H), 1.71-1.63 (m, 2H), 1.49 (d, J=16.1 Hz, 1H), 1.44 (s, 3H), 1.42-1.34 (m, 2H), 1.17 (dt, J=13.9, 6.6 Hz, 1H), 1.03 (s, 3H), 0.82 (d, J=3.8 Hz, 3H), 0.80 (d, J=3.1 Hz, 3H). ¹³C-NMR (CDCl₃, 125 MHz): δ 217.74, 170.76, 155.57, 139.84, 116.30, 76.89, 69.66, 58.86, 45.54, 45.11, 43.07, 42.24, 36.94, 36.37, 34.81, 30.54, 27.62, 27.05, 25.30, 20.99, 16.41, 15.05, 12.00. HRMS(ESI): m/z calc. for C₂₃H₃₅NO₅Na [M+Na]⁺: 428.2407, found: 428.2408.

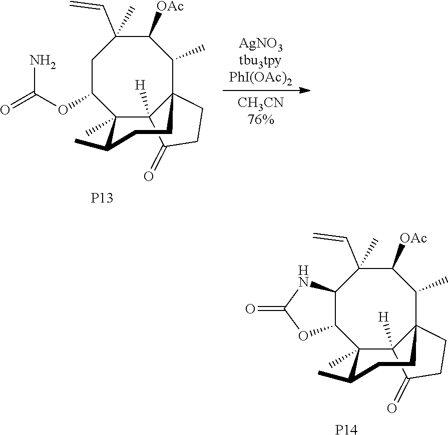

Procedure: Carbamate P13 (250 mg, 0.62 mmol) and (diacetoxyiodo)benzene (1.198 g, 3.7 mmol) were dissolved in acetonitrile and stirred for 20 min. In a separate flask, silver nitrate (11 mg, 0.062 mmol) and 4,4′,4″-tri-tert-butyl-2,2′:6′,2″-terpyridine (25 mg, 0.062 mmol) were suspended in acetonitrile (2 mL), stirred for 20 min, and then added to the flask containing carbamate P13. The combined reaction mixture was heated to reflux and stirred for 24 hours. The crude reaction was then cooled to room temperature, concentrated, and purified by flash chromatography (1:1 ethyl acetate:hexanes) to afford cyclic carbamate P14 (189 mg, 76%) as a white solid.

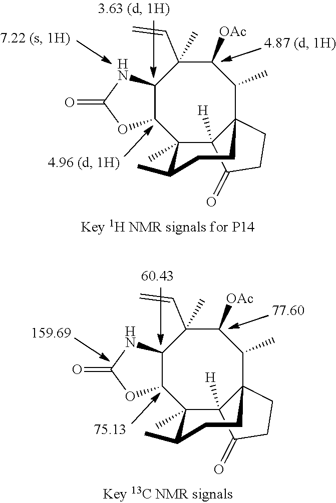

¹H-NMR (CDCl₃, 500 MHz): δ 7.22 (s, 1H), 6.00 (dd, J=18.1, 11.1 Hz, 1H), 5.43-5.38 (m, 2H), 4.96 (d, J=7.0 Hz, 1H), 4.87 (d, J=6.6 Hz, 1H), 3.63 (d, J=6.8 Hz, 1H), 2.43-2.36 (m, 1H), 2.32 (dd, J=19.6, 10.8 Hz, 1H), 2.21-2.14 (m, 1H), 2.10 (s, 3H), 1.91-1.83 (m, 1H), 1.79-1.67 (m, 3H), 1.45-1.39 (m, 3H), 1.36 (s, 3H), 1.15 (d, J=3.9 Hz, 1H), 1.10 (s, 3H), 0.93 (d, J=7.0 Hz, 3H), 0.80 (d, J=6.9 Hz, 3H). ¹³C-NMR (CDCl₃, 125 MHz): δ 216.21, 170.57, 159.69, 134.59, 120.24, 77.59, 75.13, 60.43, 57.62, 46.85, 45.46, 41.61, 36.77, 36.33, 34.47, 30.35, 27.36, 25.48, 23.98, 20.90, 17.52, 13.99, 12.49. HRMS(ESI): m/z calc. for C₂₃H₃₄NO₅ [M+H]⁺: 404.2431, found: 404.2432.

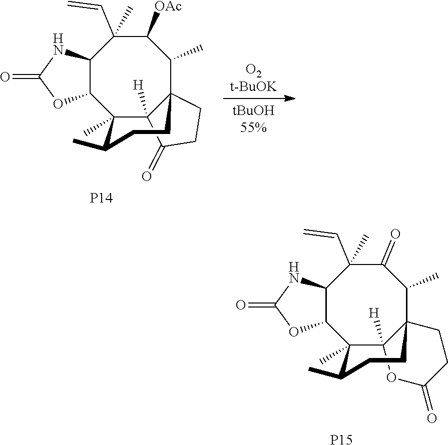

Procedure: Carbamate P14 (60 mg, 0.15 mmol) and potassium t-butoxide (168 mg, 1.5 mmol) were dissolved in t-butanol (3 mL) and stirred while bubbling oxygen through the reaction mixture for 5 minutes. The reaction was then stirred for 1 hour under ambient atmosphere then quenched by addition of 2 M hydrochloric acid. The crude mixture was then extracted with diethyl ether and washed with 0.1 M sodium hydroxide. The combined organic layers were washed with brine, dried with magnesium sulfate, and evaporated to provide lactone P15 (31 mg, 55%) as a white solid.

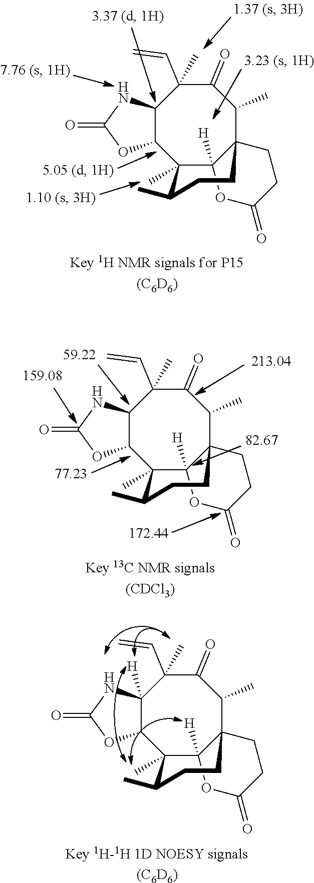

¹H-NMR (CDCl₃, 500 MHz): δ 7.30 (s, 1H), 6.34 (dd, J=17.6, 11.0 Hz, 1H), 5.49 (d, J=10.9 Hz, 1H), 5.34 (d, J=8.6 Hz, 1H), 5.18 (d, J=17.6 Hz, 1H), 3.64-3.56 (m, 2H), 3.04 (q, J=6.4 Hz, 1H), 2.68 (ddd, J=13.6, 8.6, 4.6 Hz, 1H), 2.45-2.28 (m, 2H), 1.99 (dq, J=12.8, 8.4, 6.5 Hz, 1H), 1.49 (h, J=5.1, 4.2 Hz, 4H), 1.27 (s, 3H), 1.25-1.22 (m, 1H), 1.17 (s, 3H), 1.08 (d, J=6.3 Hz, 3H), 1.00 (d, J=7.1 Hz, 3H). ¹³C-NMR (CDCl₃, 125 MHz): δ 212.78, 172.18, 158.82, 133.66, 121.38, 82.41, 77.23, 58.96, 57.11, 44.03, 43.37, 40.68, 34.58, 29.77, 26.86, 26.46, 26.07, 22.00, 17.01, 15.87, 15.39. HRMS(ESI): m/z calc. for C₂₁H₃₀NO₅ [M+H]⁺: 376.2118, found: 376.2118.

Synthesis of Derivatives for Structure-Activity Relationship Studies

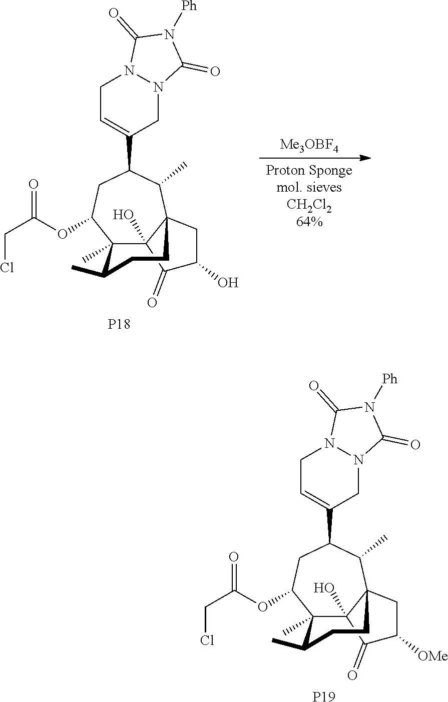

Procedure: To a solution of diol P18 (200 mg, 0.34 mmol) and proton sponge (219 mg, 1.02 mmol) in dichloromethane (7.2 mL) containing molecular sieves was added trimethyl tetrafluoroborate (75 mg, 0.51 mmol). The reaction was stirred for 5 hours at room temperature and then quenched by addition of water. The crude mixture was extracted with dichloromethane, washed with brine, dried with magnesium sulfate, and evaporated. Purification by flash chromatography (1:1 ethyl acetate:hexanes) provided methyl ether P19 (131 mg, 64%) as a white solid.

¹H-NMR (CDCl₃, 500 MHz): δ 7.53 (d, J=7.8 Hz, 2H), 7.47 (t, J=7.6 Hz, 2H), 7.37 (t, J=7.9 Hz, 1H), 5.66 (s, 1H), 5.33 (d, J=7.5 Hz, 1H), 4.27-4.09 (m, 4H), 4.03 (s, 2H), 3.61 (t, J=7.6 Hz, 1H), 3.54 (s, 3H), 3.19 (s, 1H), 2.84 (s, 1H), 2.47 (s, 1H), 2.13 (dd, J=12.5, 6.8 Hz, 1H), 2.04 (d, J=7.0 Hz, 1H), 1.95 (dd, J=12.6, 8.5 Hz, 1H), 1.80 (dq, J=12.7, 6.6, 5.7 Hz, 2H), 1.60 (d, J=4.1 Hz, 2H), 1.47 (s, 1H), 1.43 (s, 3H), 1.03-0.97 (m, 1H), 0.89 (d, J=6.9 Hz, 6H). ¹³C-NMR (C₆H₆, 151 MHz, 60° C.) δ 207.63, 165.69, 152.25 (2 C overlapping), 138.28, 132.48, 128.52, 127.83, 127.62, 127.05, 124.65, 115.57, 83.46, 78.92, 74.18, 57.99, 44.77, 43.75, 42.97, 41.46, 40.43, 37.75, 35.84, 34.85, 34.18, 26.78, 16.03, 15.63, 14.33. HRMS(ESI): m/z calc. for C₃₁H₃₇N₃O₇Cl [M−H]: 598.2320, found: 598.2318.

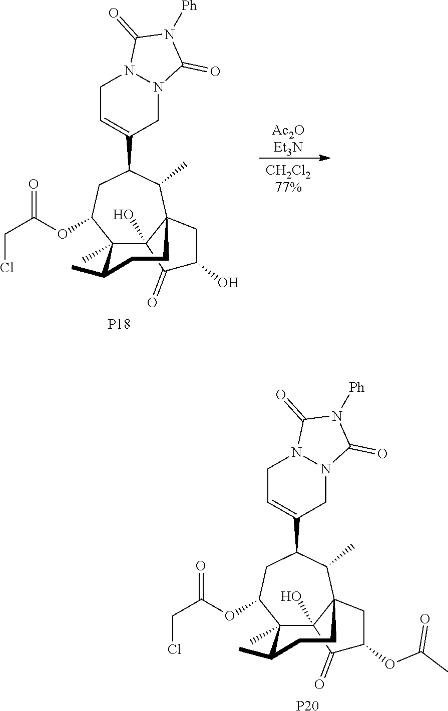

Procedure: To a solution of diol P18 (11 mg, 0.019 mmol), triethylamine (8 μL, 0.057 mmol), and NN-dimethylaminopyridine (1 mg, 0.0095 mmol) in dichloromethane (1 mL) was added acetic anhydride (6 μL, 0.057 mmol). The reaction was stirred at room temperature for 2 hours and then quenched by addition of water. The crude reaction mixture was extracted with dichloromethane, dried with magnesium sulfate, and evaporated. Purification by flash chromatography (1:1 ethyl acetate:hexanes) gave ester P20 (9 mg, 77%) as a white solid.

¹H-NMR (d7-DMF, 600 MHz, 100° C.): δ 7.65-7.60 (m, 2H), 7.56-7.50 (m, 2H), 7.45-7.39 (m, 1H), 5.85-5.80 (m, 1H), 5.48 (dd, J=10.0, 3.1 Hz, 1H), 5.43-5.38 (m, 1H), 5.15 (dd, J=8.6, 7.5 Hz, 1H), 4.36 (dd, J=2.4, 1.0 Hz, 2H), 4.33 (d, J=1.3 Hz, 1H), 4.23-4.17 (m, 1H), 4.16 (dt, J=5.7, 2.8 Hz, 2H), 3.25 (dt, J=12.6, 6.8 Hz, 1H), 2.74-2.65 (m, 1H), 2.22 (dt, J=12.2, 7.4 Hz, 2H), 2.10 (d, J=0.7 Hz, 3H), 2.08-1.99 (m, 1H), 1.84-1.72 (m, 4H), 1.51 (s, 3H), 1.51-1.45 (m, 1H), 1.24 (t, J=7.1 Hz, 1H), 0.96 (d, J=6.8 Hz, 3H), 0.91 (d, J=6.1 Hz, 3H). ¹³C NMR (151 MHz, DMF, 100° C.) δ 205.32, 169.92, 166.95, 152.62 (2 C overlapping) 138.89, 132.72, 128.90, 127.80, 125.82, 116.06, 83.40, 74.21, 72.06, 45.97, 44.31, 43.48, 43.39, 42.20, 41.47, 38.69, 37.97, 35.98, 34.97, 33.99, 27.14, 19.99, 16.23, 15.62, 14.21. HRMS(ESI): m/z calc. for C₃₂H₃₉N₃O₈Cl [M+H]⁺: 628.2420, found: 628.2410.

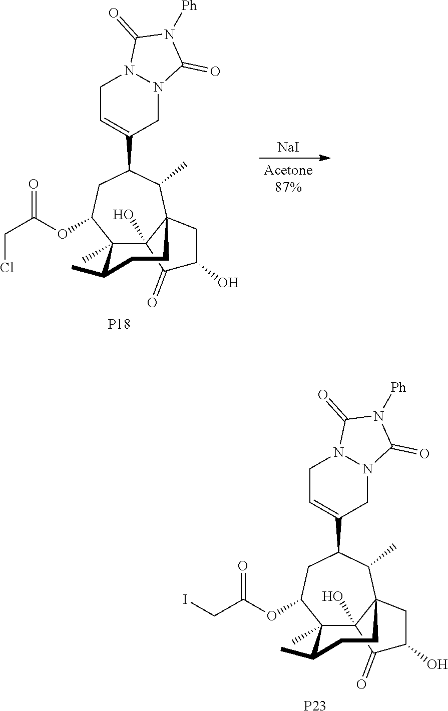

Procedure: A solution of α-chloroester P18 (106 mg, 0.18 mmol) and sodium iodide (54 mg, 3.6 mmol) in acetone was heated at 50° C. for 16 hours. The reaction was then cooled, evaporated and purified by flash chromatography (3:1 ethyl acetate:hexanes) to provide α-iodoester P23 (106 mg, 87%) as a white solid.

¹H-NMR (d7-DMF, 600 MHz, 100° C.): 68.41 (s, 1H), 7.69-7.57 (m, 2H), 7.53 (t, J=7.9 Hz, 2H), 7.47-7.37 (m, 1H), 5.86-5.81 (m, 1H), 5.50 (dd, J=10.4, 3.2 Hz, 1H), 5.40 (dd, J=10.0, 3.1 Hz, 1H), 4.89-4.75 (m, 1H), 4.34 (d, J=16.1 Hz, 1H), 4.23-4.16 (m, 1H), 4.18-4.13 (m, 2H), 3.99 (t, J=7.9 Hz, 1H), 3.93 (dd, J=44.2, 9.7 Hz, 1H), 3.26 (tt, J=13.5, 6.7 Hz, 1H), 2.69 (tt, J=13.2, 8.6 Hz, 1H), 2.21-2.14 (m, 2H), 1.90 (ddd, J=12.0, 8.4, 2.7 Hz, 1H), 1.80-1.69 (m, 3H), 1.70-1.63 (m, 1H), 1.49 (d, J=14.2 Hz, 3H), 1.46-1.39 (m, 1H), 1.18-1.09 (m, 1H), 0.95 (dd, J=6.7, 3.8 Hz, 3H), 0.89 (dd, J=15.1, 6.3 Hz, 3H). ¹³C-NMR (151 MHz, DMF, 100° C.) δ 210.50, 168.21, 167.14, 152.63, 152.62, 138.99, 132.72, 128.90, 127.80, 125.84, 115.93, 83.44, 74.00, 73.50, 71.15, 60.61, 45.44, 44.40, 43.39, 42.32, 38.53, 37.96, 36.61, 27.24, 16.26, 16.00, 15.62, 14.17. HRMS(ESI): m/z calc. for C₃₀H₃₇N₃O₇I [M+H]⁺: 678.1671, found: 678.1669.

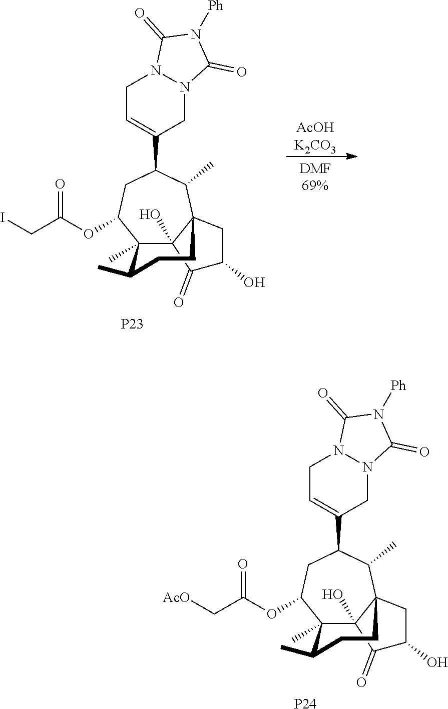

Procedure: To a solution of acetic acid (3 μL, 0.045 mmol) and potassium carbonate (19 mg, 0.14 mmol) in N,N-dimethylformamide was added α-iodoester P23 (20 mg, 0.030 mmol). The reaction was stirred at room temperature for 4 hours then quenched by addition of water. The crude mixture was extracted with ethyl acetate, washed with water and brine, dried with magnesium sulfate, and evaporated. Purification by flash chromatography (3:1 ethyl acetate:hexanes) provided ester P24 (13 mg, 69%) as a white solid.

¹H-NMR (CDCl₃, 500 MHz): δ 7.56-7.51 (m, 2H), 7.48 (td, J=8.2, 7.8, 1.9 Hz, 2H), 7.41-7.34 (m, 1H), 5.70 (s, 1H), 5.35 (d, J=8.2 Hz, 1H), 4.61 (d, J=15.9 Hz, 1H), 4.53 (d, J=15.9 Hz, 1H), 4.23-4.08 (m, 4H), 4.07-3.98 (m, 1H), 3.19-3.03 (m, 1H), 2.86 (s, 1H), 2.62 (s, 1H), 2.44-2.31 (m, 1H), 2.16 (d, J=1.8 Hz, 3H), 2.15-2.09 (m, 1H), 2.09-1.94 (m, 2H), 1.89-1.77 (m, 1H), 1.77-1.54 (m, 3H), 1.54-1.42 (m, 1H), 1.39 (d, J=1.7 Hz, 3H), 1.13-0.99 (m, 1H), 0.98-0.85 (m, 6H). ¹³C NMR (151 MHz, DMF, 100° C.) δ 210.26, 169.78, 167.41, 152.48, 152.46, 138.86, 132.57, 128.75, 127.65, 125.70, 115.76, 83.24, 73.08, 71.75, 71.02, 61.14, 61.01, 45.92, 45.30, 44.09, 43.24, 42.24, 38.28, 37.65, 36.35, 27.05, 19.51, 16.03, 15.47, 13.66. HRMS(ESI): m/z calc. for C₃₂H₄₀N₃O₉[M+H]⁺: 610.2759, found: 610.2758.

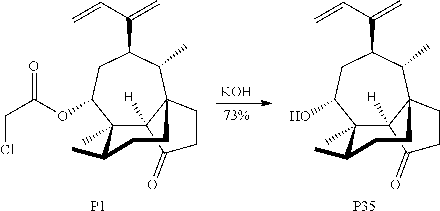

Procedure: To a solution of ester P1 (615 mg, 1.62 mmol) in ethanol (16 mL) was added potassium hydroxide (909 mg, 16.2 mmol) and the reaction was stirred at room temperature for 15 hours then quenched by addition of water. The crude mixture was extracted with ethyl acetate, washed with brine, dried with magnesium sulfate, and evaporated. Purification by flash chromatography (1:4 ethyl acetate:hexanes) afforded diol P35 (356 mg, 73%) as a white solid.

¹H-NMR (CDCl₃, 500 MHz): δ 6.30 (dd, J=17.7, 11.1 Hz, 1H), 5.44 (d, J=17.8 Hz, 1H), 5.13 (d, J=11.1 Hz, 1H), 5.05 (s, 1H), 4.89 (s, 1H), 4.28 (dd, J=12.1, 3.0 Hz, 1H), 2.39 (q, J=10.4 Hz, 1H), 2.34-2.10 (m, 3H), 2.09-1.93 (m, 2H), 1.82-1.52 (m, 5H), 1.53-1.43 (m, 2H), 1.40 (ddd, J=14.3, 9.8, 2.5 Hz, 1H), 1.30 (s, 3H), 1.15 (td, J=13.8, 5.2 Hz, 1H), 0.94 (d, J=6.8 Hz, 3H), 0.71 (d, J=6.8 Hz, 3H). ¹³C NMR (151 MHz, DMF, 100° C.) δ 216.34, 153.00, 137.53, 114.15, 113.36, 66.43, 60.55, 60.49, 45.56, 41.23, 38.53, 35.84, 35.20, 34.91, 30.24, 27.94, 25.63, 16.32, 14.48, 13.93. HRMS(ESI): m/z calc. for C₂₀H₂₉O [M−H₂O]⁺: 285.2218, found: 285.2218.

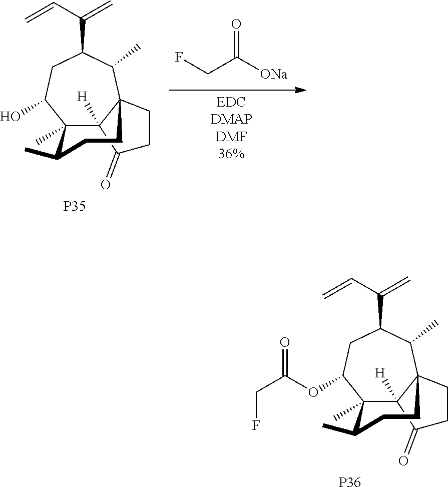

Procedure: To a solution of alcohol P35 (460 mg, 1.5 mmol), 1-Ethyl-3-(3-dimethylaminopropyl) carbodiimide hydrochloride (863 mg, 4.5 mmol), and dimethyl amino pyridine (550 mg, 4.5 mmol) in N,N-dimethylformamide (30 mL) was added sodium fluoroacetate (450 mg, 4.5 mmol). The reaction was stirred for 15 hours then quenched by addition of water. The crude reaction mixture was extracted with ethyl acetate, washed with water and brine, dried with magnesium sulfate, and evaporated. Purification by flash chromatography (1:4 ethyl acetate:hexanes) afforded α-fluoroester P36 (201 mg, 36%) as a white solid.

¹H-NMR (d7-DMF, 600 MHz, 100° C.): δ 6.45 (ddd, J=17.7, 11.1, 0.9 Hz, 1H), 5.65 (dd, J=12.4, 3.0 Hz, 1H), 5.57 (dd, J=17.7, 1.2 Hz, 1H), 5.16-5.10 (m, 2H), 5.04 (s, 1H), 4.99 (d, J=0.8 Hz, 1H), 4.91 (d, J=0.8 Hz, 1H), 2.71-2.64 (m, 1H), 2.38 (dd, J=2.8, 1.3 Hz, 1H), 2.33-2.20 (m, 3H), 2.13 (dt, J=18.9, 9.2 Hz, 1H), 1.79 (ddd, J=12.6, 10.6, 9.1 Hz, 1H), 1.71 (dqd, J=13.3, 6.7, 3.9 Hz, 1H), 1.67-1.46 (m, 4H), 1.43-1.38 (m, 4H), 1.25-1.17 (m, 1H), 0.76 (dd, J=15.2, 6.8 Hz, 6H). ¹³C NMR (151 MHz, DMF, 100° C.) δ 215.61, 167.35 (d, J_{C-F}=22.7 Hz), 152.35, 137.18, 114.61, 114.03, 79.08 (d, J_{C-F}=181.2 Hz), 77.89, 71.08, 59.37, 45.40, 40.91, 40.59, 35.63, 35.19, 34.61, 29.77, 27.67, 25.47, 15.34, 14.83, 13.80. HRMS(ESI): m/z calc. for C₂₂H₃₁O₃FNa [M+Na]⁺: 385.2149, found: 385.2155.

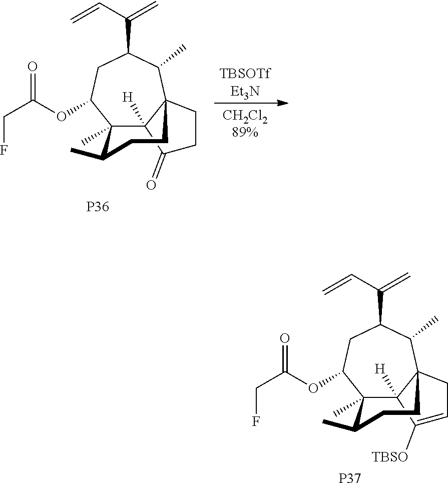

Procedure: To a solution of diene P36 (200 mg, 0.55 mmol) and triethylamine (460 μL, 3.3 mmol) in dichloromethane (9 mL) at 0° C. was added tert-butyldimethylsilyl trifluoromethanesulfonate (380 L, 1.65 mmol). The reaction was stirred for 3 hours while warming to room temperature then quenched by addition of water. The crude mixture was extracted with dichloromethane, washed with brine, dried with magnesium sulfate, and evaporated. Purification by flash chromatography (1:8 ethyl acetate: hexanes) provided silyl enol ether P37 (234 mg, 89%) as a colorless oil.

¹H-NMR (d7-DMF, 600 MHz, 80° C.): δ 6.46 (dd, J=17.7, 11.1 Hz, 1H), 5.79 (dd, J=12.2, 2.9 Hz, 1H), 5.58 (d, J=17.6 Hz, 1H), 5.14 (d, J=11.2 Hz, 1H), 5.10 (d, J=8.9 Hz, 1H), 5.04 (s, 2H), 4.96 (s, 1H), 4.56 (q, J=2.9 Hz, 1H), 2.67 (td, J=11.5, 10.9, 4.5 Hz, 1H), 2.43-2.36 (m, 1H), 2.32-2.24 (m, 1H), 2.18-2.12 (m, 2H), 1.87 (td, J=13.3, 5.7 Hz, 1H), 1.72-1.57 (m, 3H), 1.50 (t, J=5.1 Hz, 2H), 1.40 (d, J=4.2 Hz, 1H), 1.37 (s, 3H), 1.03-1.00 (m, 9H), 0.79 (d, J=6.6 Hz, 3H), 0.73 (d, J=6.8 Hz, 3H), 0.29-0.25 (m, 6H). ¹³C NMR (151 MHz, DMF, 80° C.) δ 167.52 (d, J_{C}-F=19.6 Hz), 157.37, 137.11, 114.52, 113.78, 99.75, 77.93 (d, J_{C}-F=178.2 Hz), 71.63, 53.43, 48.46, 43.36, 40.57, 36.34, 35.15, 31.81, 28.07, 25.87, 25.65, 18.31, 16.83, 15.74, 14.84, −3.62, −5.18. HRMS(ESI): m/z calc. for C₂₈H₄₆O₃FSi [M+H]⁺: 477.3195, found: 477.3191.

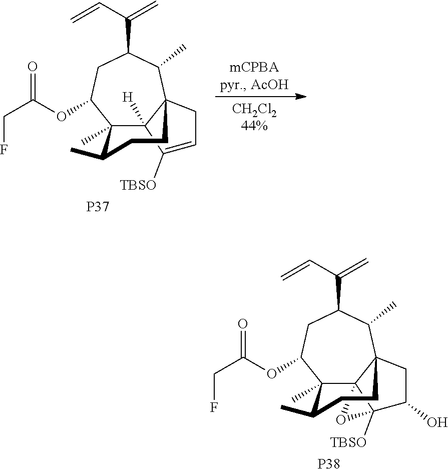

Procedure: To a solution of m-chloroperoxybenzoic acid (339 mg, 1.47 mmol), pyridine (218 μL, 2.70 mmol), and glacial acetic acid (647 μL, 11.3 mmol) in dichloromethane (4 mL) at −78° C. was added a solution of silyl enol ether P37 (234 mg, 0.49 mmol) in dichloromethane (0.9 mL). The reaction was stirred at −78° C. for one hour then warmed to 0° C. and stirred an additional 3 hours while allowing the reaction to warm to room temperature. The reaction was quenched by addition of a saturated sodium sulfite solution and by addition of a solution of saturated sodium bicarbonate. The reaction mixture was then extracted with dichloromethane, washed with brine, and dried with magnesium sulfate. Purification of crude mixture was accomplished by adsorption onto silica by evaporation and flash chromatography (1:7 ethyl acetate:hexanes) to provide a single isomer of epoxide P38 (110 mg, 44%) as a white foam.

¹H-NMR (d7-DMF, 600 MHz, 80° C.): δ 6.46 (ddd, J=17.6, 11.1, 0.8 Hz, 1H), 5.83 (dd, J=12.3, 2.6 Hz, 1H), 5.49 (d, J=17.6 Hz, 1H), 5.14-5.01 (m, 3H), 4.99-4.96 (m, 1H), 4.56 (d, J=8.0 Hz, 1H), 4.22 (d, J=7.5 Hz, 1H), 2.89-2.83 (m, 1H), 2.69-2.60 (m, 1H), 2.30-2.24 (m, 1H), 1.75-1.65 (m, 3H), 1.58-1.49 (m, 3H), 1.39-1.29 (m, 3H), 1.23 (s, 3H), 0.96 (s, 9H), 0.83 (s, 3H), 0.61 (d, J=6.7 Hz, 3H), 0.33 (s, 3H), 0.32-0.27 (m, 3H). ¹³C NMR (151 MHz, DMF, 80° C.) δ 167.35 (d, J_{C}-F=22.7 Hz), 152.64, 137.41, 114.35, 113.47, 89.69, 78.51 (d, J_{C}-F=181.2 Hz), 74.94, 71.91, 70.85, 45.58, 44.04, 42.19, 36.74, 35.46, 34.15, 27.36, 25.93, 25.69, 18.25, 15.76, 13.82, 13.60, −3.34, −3.69. HRMS(ESI): m/z calc. for C₂₈H₄₆O₅FSi [M+H]⁺: 509.3093, found: 509.3085.

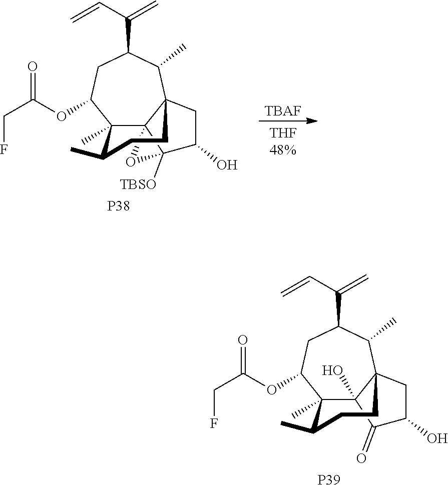

Procedure: A solution of epoxide P38 (107 mg, 0.21 mmol) and tetrabutylammonium fluoride (0.42 mL, 0.42 mmol, 1.0 M in THF) in tetrahydrofuran (4.2 mL) stirred at room temperature for 3 hours and then quenched by addition of a solution of sodium iodide in acetone and water. The crude mixture was extracted with ethyl acetate and the combined organic layers were washed with brine, dried with magnesium sulfate, and evaporated. Purification by flash chromatography (3:2 ethyl acetate:hexanes) gave diol P39 (39 mg, 48%) as a white solid.

¹H NMR (DMSO-d₆, 600 MHz, 80° C.) δ 5.60 (ddd, J=17.5, 11.0, 0.9 Hz, 1H), 4.78 (dd, J=10.1, 3.3 Hz, 1H), 4.64 (dd, J=17.4, 1.4 Hz, 1H), 4.29-4.23 (m, 1H), 4.23-4.18 (m, 1H), 4.13-4.10 (m, 1H), 4.10-3.99 (m, 1H), 4.02-3.91 (m, 1H), 3.46 (d, J=1.9 Hz, 1H), 3.13 (dd, J=8.5, 7.6 Hz, 1H), 2.53-2.47 (m, 1H), 1.71 (ddd, J=14.5, 10.1, 7.7 Hz, 1H), 1.36-1.27 (m, 2H), 1.07 (dd, J=12.3, 8.6 Hz, 1H), 0.97-0.85 (m, 3H), 0.81 (ddd, J=14.9, 4.5, 2.2 Hz, 1H), 0.63 (dtd, J=7.2, 5.0, 2.7 Hz, 1H), 0.60 (s, 3H), 0.32-0.25 (m, 1H), 0.04 (dd, J=6.7, 5.3 Hz, 6H). ¹³C-NMR (151 MHz, DMSO, 80° C.) δ 203.09, 160.03 (d, J_{C-F}=22.7 Hz), 145.81, 129.81, 105.66, 104.71, 75.80, 70.11 (d, J_{C-F}=176.7 Hz), 66.35, 63.38, 37.59, 36.30, 31.87, 31.31, 30.72, 29.30, 28.18, 27.16, 19.27, 7.58, 7.16, 5.74. HRMS(ESI): m/z calc. for C₂₂H₃₀O₅F [M−H]⁻: 393.2077, found: 393.2072.

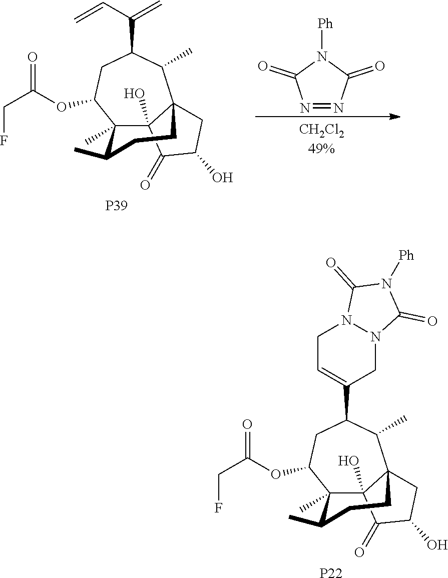

Procedure: To a solution of diene P39 (8 mg, 0.02 mmol) in dichloromethane (0.4 mL) was added 4-Phenyl-1,2,4-triazole-3,5-dione (4 mg, 0.24 mmol). The reaction was stirred 3 hours at room temperature then quenched by addition of water. The crude mixture was extracted with dichloromethane, dried with magnesium sulfate, and evaporated. Purification by flash chromatography (3:2-3:1 ethyl acetate:hexanes) provided urazole P22 (6 mg, 49%) as a white solid.

¹H-NMR (d7-DMF, 600 MHz, 100° C.): δ 7.66-7.59 (m, 2H), 7.57-7.50 (m, 2H), 7.46-7.39 (m, 1H), 5.83 (dd, J=3.8, 2.0 Hz, 1H), 5.55 (dd, J=10.4, 3.1 Hz, 1H), 5.10-4.96 (m, 3H), 4.96-4.91 (bs, 1H), 4.37 (dd, J=16.6, 2.5 Hz, 1H), 4.23-4.17 (m, 1H), 4.18-4.14 (m, 2H), 3.99 (t, J=8.0 Hz, 1H), 3.30-3.22 (m, 1H), 2.76-2.70 (m, 1H), 2.18 (ddd, J=24.6, 12.3, 7.0 Hz, 2H), 1.90 (dd, J=12.2, 8.4 Hz, 1H), 1.82-1.72 (m, 3H), 1.71-1.64 (m, 1H), 1.50-1.46 (m, 3H), 1.46-1.41 (m, 1H), 1.18-1.09 (m, 1H), 0.95 (d, J=6.8 Hz, 3H), 0.87 (d, J=6.2 Hz, 3H). ¹³C NMR (151 MHz, DMF, 100° C.) δ 210.34, 167.60 (d, J_{C}-F=22.7 Hz), 152.63 (2 C), 139.00, 132.72, 128.90, 127.80, 125.83, 115.93, 83.36, 78.51 (d, J_{C}-F=181.2 Hz), 73.19, 71.15, 45.46, 44.25, 43.44, 43.39, 42.41, 38.40, 37.64, 36.49, 35.94, 34.85, 27.18, 16.14, 15.57, 13.74. HRMS(ESI): m/z calc. for C₃₀H₃₇N₃O₇F [M+H]⁺: 570.2610, found: 570.2610.

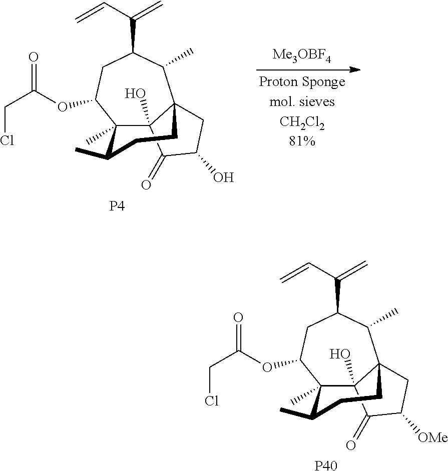

Procedure: To a solution of diol P4 (470 mg, 1.14 mmol) and proton sponge (366 mg, 1.70 mmol) in dichloromethane (22.8 mL) containing molecular sieves was added trimethyl tetrafluoroborate (180 mg, 1.26 mmol). The reaction was stirred for 3 hours at room temperature and then quenched by addition of water. The crude mixture was extracted with dichloromethane, washed with brine, dried with magnesium sulfate, and evaporated. Purification by flash chromatography (1:3 ethyl acetate:hexanes) provided methyl ether P40 (393 mg, 81%) as a white solid.

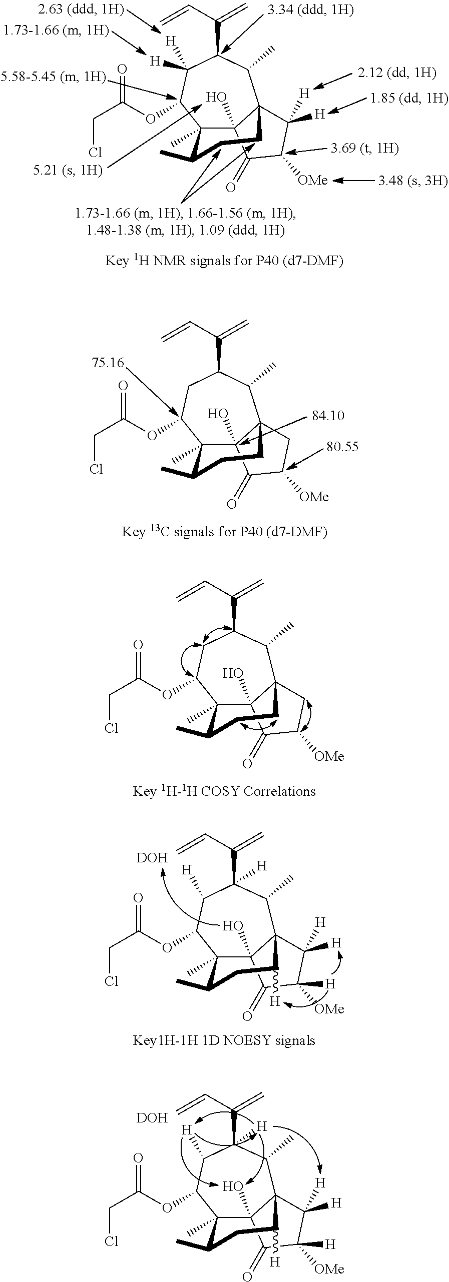

¹H-NMR (d7-DMF, 500 MHz, 80° C.): δ 6.47 (dd, J=17.5, 11.0 Hz, 1H), 5.58-5.45 (m, 2H), 5.21 (s, 1H), 5.12 (d, J=11.0 Hz, 1H), 5.08 (s, 1H), 4.98 (s, 1H), 4.36 (d, J=14.4 Hz, 1H), 4.31 (d, J=14.6 Hz, 1H), 3.69 (t, J=7.9 Hz, 1H), 3.48 (s, 3H), 3.34 (ddd, J=12.9, 8.3, 5.8 Hz, 1H), 2.63 (ddd, J=14.5, 10.7, 8.3 Hz, 1H), 2.23-2.15 (m, 1H), 2.12 (dd, J=12.2, 7.5 Hz, 1H), 1.85 (dd, J=12.1, 8.3 Hz, 1H), 1.77 (dtd, J=13.4, 6.6, 4.0 Hz, 1H), 1.73-1.66 (m, 2H), 1.66-1.56 (m, 1H), 1.48 (d, J=2.1 Hz, 3H), 1.48-1.38 (m, 1H), 1.09 (ddd, J=15.0, 12.5, 5.0 Hz, 1H), 0.85 (d, J=7.1 Hz, 3H), 0.84 (d, J=7.2 Hz, 3H). ¹³C-NMR (d7-DMF, 500 MHz, 80° C.): δ 209.36, 167.73, 154.72, 138.71, 115.19, 113.97, 84.10, 80.55, 75.16, 58.65, 46.27, 45.22, 42.51, 40.95, 39.34, 37.90, 37.86, 35.72, 34.64, 28.19, 16.75, 16.64, 14.74. HRMS(ESI): m/z calc. for C₂₃H₃₃O₅ClNa [M+Na]⁺: 447.1909, found: 447.1904.

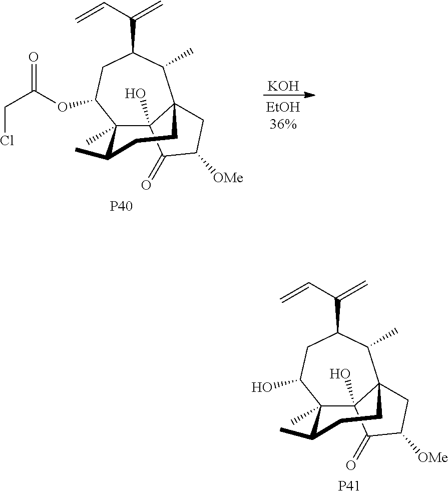

Procedure: To a solution of ester P40 (356 mg, 0.84 mmol) in ethanol (8.4 mL) was added potassium hydroxide (235 mg, 4.1 mmol) and the reaction was stirred at room temperature for 12 hours then quenched by addition of saturated aqueous ammonium chloride. The crude mixture was extracted with ethyl acetate, washed with brine, dried with magnesium sulfate, and evaporated. Purification by flash chromatography (1:3 ethyl acetate:hexanes) gave diol P41 (107 mg, 36%) as a white solid.

¹H-NMR (d7-DMF, 600 MHz, 100° C.): δ 6.41 (dd, J=17.5, 11.0 Hz, 1H), 5.88 (s, 1H), 5.53-5.46 (m, 1H), 5.09 (dd, J=6.4, 4.6 Hz, 2H), 5.04-4.95 (m, 2H), 4.02-3.92 (m, 1H), 3.67 (t, J=8.3 Hz, 1H), 3.46 (s, 2H), 3.39 (ddd, J=11.5, 9.8, 3.7 Hz, 1H), 2.25 (dd, J=12.3, 8.1 Hz, 1H), 2.19 (ddd, J=14.9, 6.5, 3.7 Hz, 1H), 2.10 (ddd, J=14.8, 9.8, 2.5 Hz, 1H), 2.02 (dq, J=11.6, 7.1 Hz, 1H), 1.92 (dd, J=12.2, 8.4 Hz, 1H), 1.77 (qd, J=13.4, 4.3 Hz, 1H), 1.67-1.59 (m, 1H), 1.55 (s, 3H), 1.54-1.48 (m, 1H), 1.42-1.33 (m, 2H), 1.15 (ddd, J=14.0, 11.6, 4.8 Hz, 1H), 0.96 (dd, J=8.6, 7.0 Hz, 6H). ¹³C NMR (151 MHz, DMF, 100° C.) δ 210.44, 154.76, 138.64, 113.54, 112.39, 85.25, 79.41, 78.26, 73.62, 57.27, 45.69, 44.03, 43.01, 42.39, 39.58, 38.53, 37.25, 35.42, 28.11, 17.41, 15.53. HRMS(ESI): m/z calc. for C₂₁H₃₂O₄Na [M+Na]⁺: 371.2193 found: 371.2190.

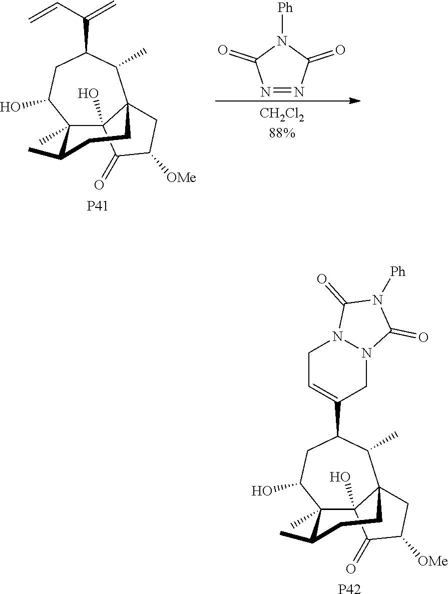

Procedure: Diene P41 (85 mg, 0.24 mmol) and 4-phenyl-1,2,4-triazole-3,5-dione (51 mg, 0.29 mmol) were dissolved in dichloromethane (2.4 mL) and stirred for 7 hours. The reaction was quenched by addition of water and extracted with dichloromethane. The combined organic layers were washed with brine, dried with magnesium sulfate, and evaporated. Purification by flash chromatography (2:1 ethyl acetate:hexanes) afforded urazole P42 (111 mg, 88%) as a white solid.

¹H-NMR (d7-DMF, 600 MHz, 100° C.): δ 7.66-7.59 (m, 2H), 7.53 (dd, J=8.5, 7.2 Hz, 2H), 7.48-7.37 (m, 1H), 5.96 (s, 1H), 5.81 (s, 1H), 5.11 (s, 1H), 4.18 (dq, J=8.7, 3.0 Hz, 4H), 4.04 (ddd, J=13.8, 9.1, 3.9 Hz, 2H), 3.53 (s, 2H), 3.19 (ddd, J=12.4, 9.4, 3.8 Hz, 1H), 2.81 (dd, J=13.3, 10.2 Hz, 1H), 2.34 (ddd, J=14.7, 6.9, 3.8 Hz, 1H), 2.15-2.04 (m, 2H), 1.99-1.90 (m, 1H), 1.83-1.73 (m, 1H), 1.50 (s, 3H), 1.46-1.34 (m, 4H), 1.27 (dd, J=13.3, 2.0 Hz, 1H), 0.98 (dd, J=7.1, 4.6 Hz, 6H). ¹³C NMR (151 MHz, DMF, 100° C.) δ 211.02, 152.67, 152.59, 139.30, 132.72, 128.91, 127.82, 125.83, 115.27, 87.67, 78.25, 72.87, 58.60, 58.45, 46.50, 43.91, 43.66, 43.39, 41.29, 40.84, 40.67, 38.23, 36.08, 28.04, 18.54, 16.89, 15.56. HRMS(ESI): m/z calc. for C₂₉H₃₈N₃O₆ [M+H]⁺: 524.2755, found: 524.2755.

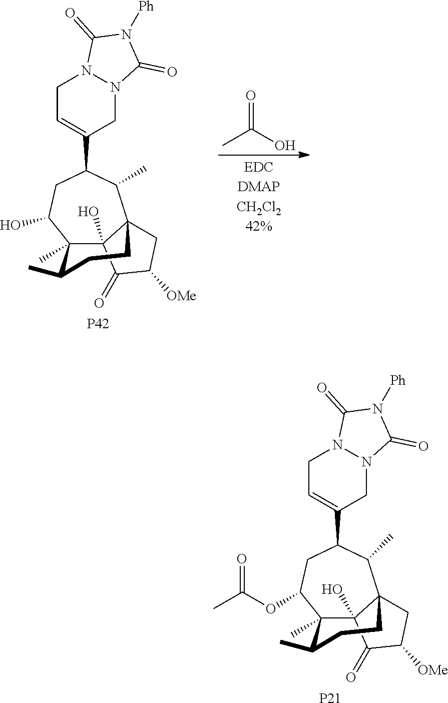

Procedure: A solution of diol P42 (20 mg, 0.04 mmol), acetic acid (7 μL, 0.12 mmol), 1-Ethyl-3-(3-dimethylaminopropyl)carbodiimide hydrochloride (23 mg, 0.12 mmol), and dimethyl amino pyridine (15 mg, 0.12 mmol) in dichloromethane (0.4 mL) was stirred for 26 hours then quenched by addition of water. The crude reaction mixture was extracted with dichloromethane, dried with magnesium sulfate, and evaporated. Purification by flash chromatography (3:2 ethyl acetate:hexanes) provided ester P21 (9 mg, 42%) as a white solid.

¹H-NMR (d7-DMF, 600 MHz, 100° C.): δ 7.62 (dd, J=7.9, 1.4 Hz, 2H), 7.53 (t, J=7.9 Hz, 2H), 7.42 (dd, J=8.2, 6.6 Hz, 1H), 5.80 (s, 1H), 5.41 (dd, J=11.4, 3.2 Hz, 1H), 5.29 (s, 1H), 4.56-4.30 (m, 1H), 4.25-4.01 (m, 4H), 3.54 (s, 3H), 3.13 (d, J=10.3 Hz, 1H), 2.68 (dd, J=13.1, 9.7 Hz, 1H), 2.27 (dq, J=13.4, 6.8 Hz, 1H), 2.03 (s, 4H), 1.69 (qd, J=13.5, 4.6 Hz, 1H), 1.58 (ddd, J=19.8, 14.7, 4.6 Hz, 2H), 1.53-1.28 (m, 6H), 1.21 (dd, J=13.2, 1.7 Hz, 1H), 0.88 (d, J=6.8 Hz, 3H), 0.82 (d, J=6.8 Hz, 3H). ¹³C NMR (151 MHz, DMF, 100° C.) δ 209.22, 169.97, 152.65, 152.61, 138.90, 132.73, 128.89, 127.79, 125.83, 115.85, 85.23, 79.12, 79.09, 70.98, 58.61, 58.59, 46.80, 43.68, 43.38, 42.46, 37.28, 34.55 33.57, 27.13, 20.75, 20.72, 15.52, 15.09, 12.92. HRMS(ESI): m/z calc. for C₃₁H₄₀N₃O₇ [M+H]⁺: 566.2861, found: 566.2859.

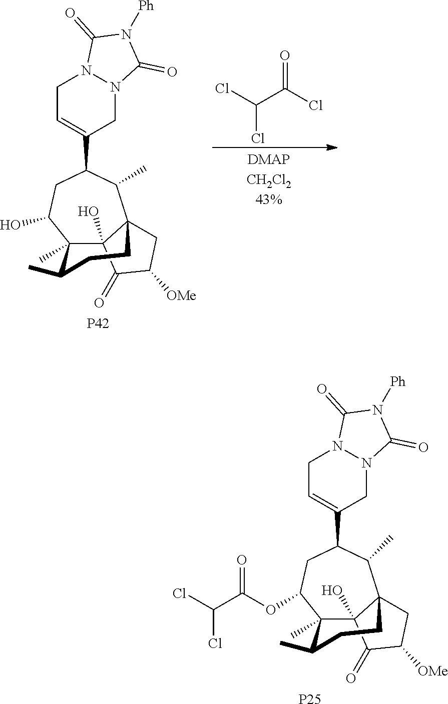

Procedure: To a solution of diol P42 (20 mg, 0.04 mmol) and dimethyl amino pyridine (15 mg, 0.12 mmol) in dichloromethane (0.4 mL) was added dichloroacetyl chloride (12 μL, 0.12 mmol). The reaction was stirred for 19 hours then quenched by addition of water. The crude reaction mixture was extracted with dichloromethane, dried with magnesium sulfate, and evaporated. Purification by flash chromatography (2:1 ethyl acetate:hexanes) afforded dichloro ester P25 (11 mg, 43%) as a white solid.

¹H-NMR (d7-DMF, 600 MHz, 100° C.): δ 7.67-7.58 (m, 2H), 7.53 (td, J=8.0, 1.7 Hz, 2H), 7.46-7.38 (m, 1H), 5.91-5.79 (m, 1H), 5.58-5.39 (m, 2H), 4.42-4.30 (m, 1H), 4.24 (dt, J=17.3, 2.0 Hz, 1H), 4.21-4.14 (m, 3H), 3.55 (d, J=2.8 Hz, 3H), 3.29-3.15 (m, 1H), 2.76-2.68 (m, 1H), 2.28 (ddd, J=14.9, 12.4, 6.9 Hz, 1H), 2.11 (dddd, J=22.2, 12.3, 6.9, 4.8 Hz, 1H), 1.83-1.57 (m, 3H), 1.51-1.40 (m, 5H), 1.34 (d, J=4.4 Hz, 1H), 1.25 (ddd, J=13.1, 6.2, 1.8 Hz, 1H), 0.92 (d, J=6.7 Hz, 3H), 0.91-0.88 (m, 3H). ¹³C NMR (151 MHz, DMF, 100° C.) δ 209.15, 163.89, 160.96, 152.66, 138.39, 132.71, 128.89, 127.80, 125.82, 116.55, 116.30, 85.11, 79.06, 75.84, 65.77, 58.62, 46.87, 46.83, 44.66, 44.33, 43.39, 41.96, 37.26, 35.97, 33.60, 26.97, 15.85, 15.20, 13.25. HRMS(ESI): m/z calc. for C₃₁H₃₆N₃O₇Cl₂ [M−H]⁻:632.1930, found: 632.1924.

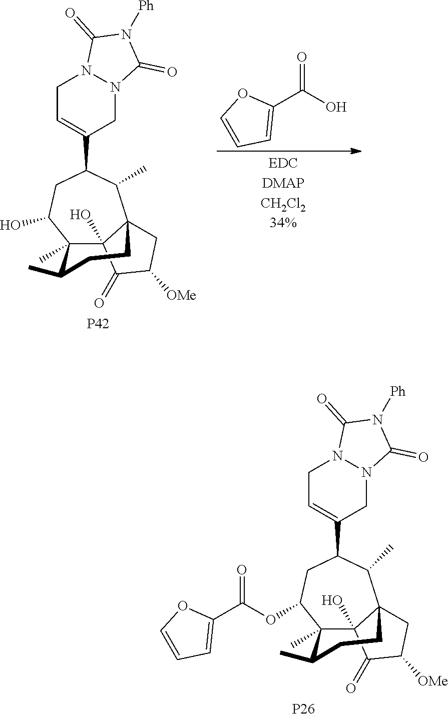

Procedure: A solution of diol P42 (20 mg, 0.04 mmol), furoic acid (14 mg, 0.12 mmol), 1-Ethyl-3-(3-dimethylaminopropyl)carbodiimide hydrochloride (23 mg, 0.12 mmol), and dimethyl amino pyridine (15 mg, 0.12 mmol) in dichloromethane (0.4 mL) was stirred for 24 hours then quenched by addition of water. The crude reaction mixture was extracted with dichloromethane, dried with magnesium sulfate, and evaporated. Purification by flash chromatography (2:3 ethyl acetate:hexanes) afforded furoic ester P26 (8 mg, 34%) as a white solid.

¹H-NMR (d7-DMF, 600 MHz, 100° C.): δ 7.90 (q, J=1.2 Hz, 1H), 7.62 (dd, J=8.0, 1.9 Hz, 2H), 7.53 (td, J=7.9, 1.6 Hz, 2H), 7.46-7.39 (m, 1H), 7.29 (d, J=3.5 Hz, 1H), 6.68 (dt, J=3.3, 1.6 Hz, 1H), 5.82 (s, 1H), 5.62 (dd, J=11.1, 3.1 Hz, 1H), 5.42 (s, 1H), 4.60-4.42 (m, 1H), 4.33-4.07 (m, 4H), 3.55 (d, J=1.6 Hz, 3H), 3.20 (t, J=10.8 Hz, 1H), 2.92-2.83 (m, 1H), 2.75-2.67 (m, 1H), 2.33 (dq, J=13.3, 6.9 Hz, 1H), 2.11 (dt, J=12.6, 6.6 Hz, 1H), 1.80-1.66 (m, 2H), 1.63 (dd, J=15.1, 4.5 Hz, 1H), 1.51 (d, J=1.6 Hz, 3H), 1.50-1.37 (m, 2H), 1.24 (dd, J=12.9, 2.0 Hz, 1H), 0.95-0.88 (m, 3H), 0.83 (dd, J=6.9, 1.8 Hz, 3H). ¹³C NMR (151 MHz, DMF, 100° C.) δ 209.19, 157.86, 152.64 (2 C), 147.43, 145.52, 138.80, 132.75, 128.89, 127.78, 125.85, 117.95, 116.03, 112.12, 85.20, 79.14, 79.08, 71.94, 58.62, 46.88, 44.13, 43.37, 42.45, 37.28, 35.71, 35.32, 34.57, 33.56, 27.12, 15.65, 15.10, 13.04. HRMS(ESI): m/z calc. for C₃₄H₄₀N₃O₈[M+H]⁺: 618.2810, found: 618.2813.

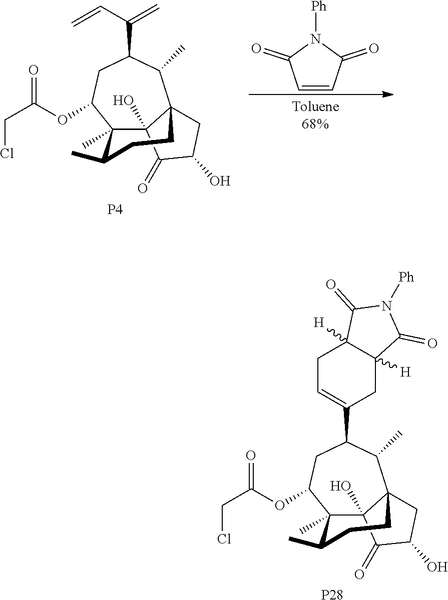

Procedure: A solution of diene P4 (20 mg, 0.05 mmol) and N-phenylmaleimide (26 mg, 0.15 mmol) in toluene (1 mL) was heated at 60° C. in a sealed vial for 20 hours. The reaction was then cooled and directly purified by flash chromatography (3:2 ethyl acetate:hexanes) to afford succinimide P28 (19 mg, 68%, white solid) as a mixture of diastereomers. Note: Compound P29 exists and is presented as a mixture of diastereomers.

¹H NMR (600 MHz, DMF-d₇, 100° C.): δ 7.50 (td, J=7.8, 3.0 Hz, 3H), 7.42 (ddt, J=7.3, 5.5, 1.3 Hz, 1H), 7.37-7.30 (m, 2H), 7.30-7.24 (m, 1H), 5.68 (t, J=5.2 Hz, 1H), 5.63 (dd, J=6.3, 3.2 Hz, 1H), 5.57 (dd, J=9.8, 3.1 Hz, 1H), 5.49 (dd, J=10.1, 3.1 Hz, 1H), 4.89-4.73 (m, 2H), 4.39-4.18 (m, 3H), 3.97 (q, J=8.0 Hz, 2H), 3.47-3.25 (m, 4H), 3.20-3.03 (m, 2H), 2.75-2.58 (m, 2H), 2.53 (dddd, J=18.7, 12.2, 10.7, 6.6 Hz, 4H), 2.46-2.32 (m, 4H), 2.19-1.95 (m, 5H), 1.85 (ddd, J=16.9, 12.2, 8.4 Hz, 2H), 1.78-1.51 (m, 8H), 1.51-1.35 (m, 9H), 1.13 (qd, J=14.1, 4.9 Hz, 2H), 0.92-0.74 (m, 12H). ¹³C NMR (151 MHz, DMF-dy, 100° C.) δ 211.95, 211.89, 180.35, 180.33, 180.13, 167.95, 167.64, 146.67, 134.73, 134.69, 130.06, 130.03, 129.36, 129.32, 128.17, 128.14, 128.11, 121.99, 121.77, 84.78, 84.64, 75.84, 75.64, 72.43, 46.75, 46.56, 45.62, 45.59, 45.50, 45.00, 42.69, 42.63, 41.26, 41.09, 40.89, 40.70, 39.80, 39.68, 39.52, 37.92, 37.17, 36.54, 36.55, 30.81, 28.55, 28.48, 25.04, 24.95, 17.96, 17.93, 16.83, 16.65, 15.55, 15.25. HRMS(ESI): m/z calc. for C₃₂H₃₉NO₇Cl [M+H]⁺: 584.2410, found: 584.2410.

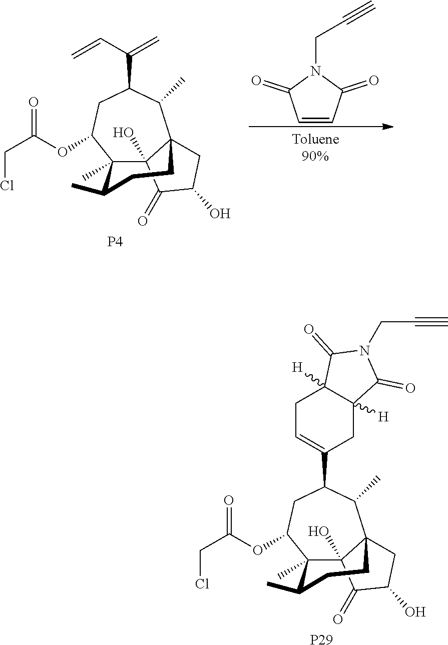

Procedure: Diene P4 (30 mg, 0.073 mmol) and N-propargyl maleimide were dissolved in toluene (0.73 mL) heated to 80° C. in a sealed vial for 19 hours. The reaction was the cooled to room temperature and purified by flash chromatography (3:2 ethyl acetate:hexanes) to provide alkyne P29 (35.7 mg, 90%, white solid) as a mixture of diastereomers (dr=1:1). Not: Compound P29 exists and is presented as a mixture of diastereomers.

¹H NMR (600 MHz, DMF-d₇, 100° C.): 5.62-5.51 (m, 3H), 5.45 (dd, J=10.1, 3.1 Hz, 1H), 4.84 (t, J=18.1 Hz, 2H), 4.37-4.29 (m, 3H), 4.23-4.15 (m, 4H), 3.96 (dd, J=10.8, 5.0 Hz, 2H), 3.31-3.13 (m, 4H), 3.09-2.97 (m, 5H), 2.89-2.84 (m, 1H), 2.84-2.80 (m, 1H), 2.68-2.47 (m, 3H), 2.46-2.26 (m, 7H), 2.16-1.99 (m, 5H), 1.84 (dddd, J=12.1, 8.6, 6.7, 2.5 Hz, 2H), 1.78-1.66 (m, 4H), 1.66-1.54 (m, 4H), 1.48-1.42 (m, 7H), 1.12 (ddd, J=13.4, 10.2, 5.0 Hz, 2H), 0.90-0.83 (m, 6H), 0.82-0.76 (m, 3H), 0.76-0.68 (m, 3H). ¹³C NMR (151 MHz, DMF, 100° C.) δ 210.76, 210.64, 178.75, 178.63, 178.43, 166.74, 166.48, 145.74, 145.32, 120.59, 120.56, 120.40, 120.37, 83.53, 83.43, 77.89, 77.80, 74.70, 74.24, 72.21, 72.16, 72.04, 72.00, 71.22, 45.53, 45.35, 44.35, 44.28, 43.76, 41.54, 41.52, 39.87, 39.74, 39.50, 39.32, 38.53, 38.48, 38.03, 36.76, 36.57, 36.00, 35.65, 35.20, 27.55, 27.46, 27.30, 23.58, 23.49, 16.78, 16.63, 15.62, 15.46, 14.13, 14.06. HRMS(ESI): m/z calc. for C₂₉H₃₆NO₇ClNa [M+Na]⁺: 568.2073, found: 568.2084.

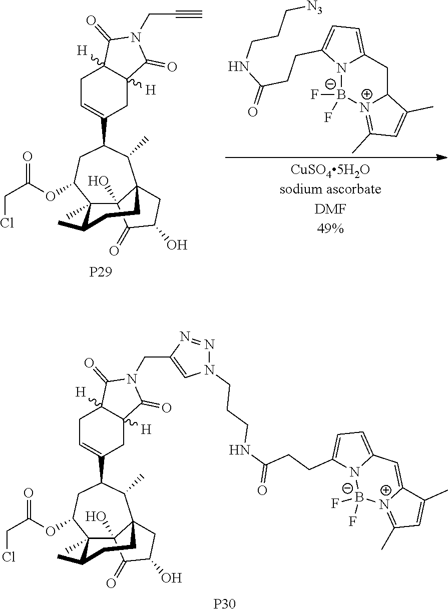

Procedure: To a solution of alkyne P29 (3 mg, 0.005 mmol) and BDP FL azide (2 mg, 0.005 mmol) in N,N-dimethylformamide (0.3 mL) were added sodium ascorbate (1 mg, 0.005 mmol) and copper sulfate pentahydrate (1 mg, 0.003 mmol). The reaction was stirred in darkness for 24 hours then quenched by addition of water. The crude mixture was extracted with ethyl acetate, washed with water, dried with magnesium sulfate, and dry loaded onto silica by evaporation. Purification by flash chromatography (1% methanol in ethyl acetate) provided fluorescent probe P30 (2.4 mg, 49%) as a mixture of diastereomers. Note: Compound P30 exists as a mixture of diastereomers and >100 protons are present in the ¹H NMR spectrum. HRMS(ESI): m/z calc. for C₄₆H₅₈N₇O₅ClBF₂ [M+H]⁺: 920.4091, found: 920.4105.

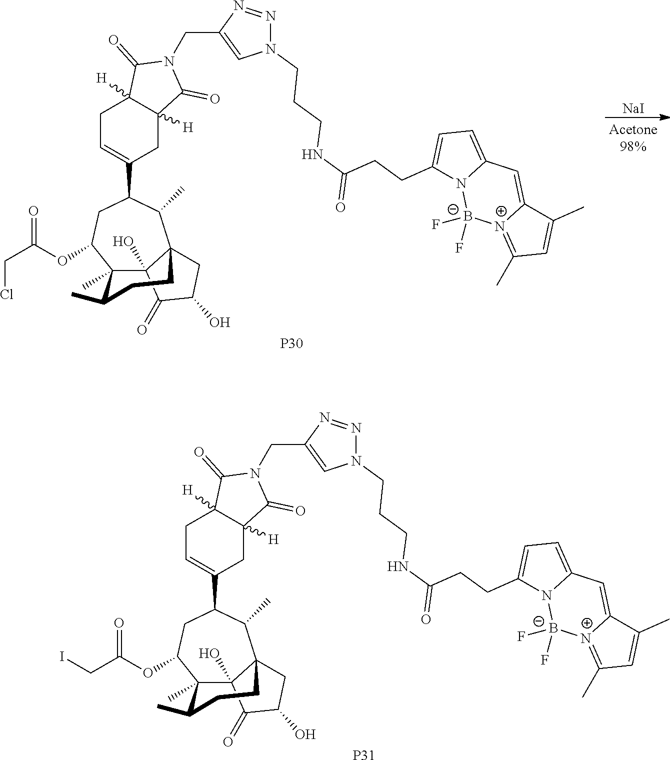

Procedure: To a solution of P30 (1 mg, 0.001 mmol), NaI (4 mg, 0.02 mmol) in acetone (0.2 mL) was added. The reaction was heated at 40° C. while stirring in darkness for 4 hours then quenched by addition of water. The crude mixture was extracted with dichloromethane and isopropanol, and dry loaded onto silica by evaporation. Purification by flash chromatography (1% methanol in ethyl acetate) provided fluorescent probe P31 (1 mg, 98%) as a mixture of diastereomers. Note: Compound P31 exists as a mixture of diastereomers and >100 protons are present in the ¹H NMR spectrum.

HRMS(ESI): m/z calc. for C₄₆H₅₈N₇O₈IBF₂ [M+H]⁺: 1012.3447, found: 1012.3467.

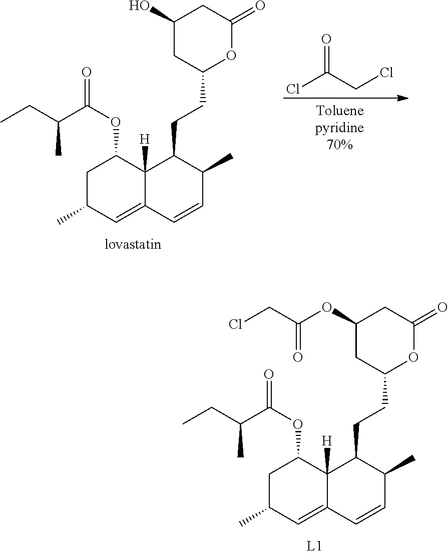 [SMILES: CC[C@H](C)C(=O)O[C@H]1C[C@@H](C)C=C2C=C[C@H](C)[C@H](CC[C@@H]3C[C@@H](O)CC(=O)O3)[C@H]21 ; O=C(Cl)CCl ; CC[C@H](C)C(=O)O[C@H]1C[C@@H](C)C=C2C=C[C@H](C)[C@H](CC[C@@H]3C[C@@H](OC(=O)CCl)CC(=O)O3)[C@H]21]

Procedure: To a solution of lovastatin (100 mg, 0.25 mmol) in toluene (2.5 mL) at 0° C., chloromethyl chloride (40 μL, 0.5 mmol) in pyridine (0.44 μL) was added. The reaction was stirred from 0° C. to r.t. for 18 hours and then quenched by addition of water. The crude mixture was extracted with ethyl acetate, and dry loaded onto silica by evaporation. Purification by flash chromatography (2:1 ethyl acetate in hexane) provided compound L1 (84.2 mg, 70%).

¹H NMR (CDCl₃, 500 MHz) δ 5.96 (d, J=9.6 Hz, 1H), 5.75 (dd, J=9.6, 6.0 Hz, 1H), 5.50 (t, J=3.2 Hz, 1H), 5.38-5.27 (m, 2H), 4.51-4.39 (m, 1H), 4.06 (s, 2H), 2.81 (dd, J=18.1, 5.4 Hz, 1H), 2.71 (ddd, J=18.1, 3.5, 1.7 Hz, 1H), 2.41 (tq, J=10.2, 6.8, 5.0 Hz, 1H), 2.37-2.27 (m, 2H), 2.24 (dq, J=12.1, 2.8 Hz, 1H), 2.08 (dtd, J=14.9, 3.2, 1.8 Hz, 1H), 1.98-1.84 (m, 2H), 1.84-1.71 (m, 2H), 1.71-1.56 (m, 2H), 1.53-1.33 (m, 3H), 1.33-1.20 (m, 1H), 1.06 (dd, J=15.6, 7.2 Hz, 6H), 0.87-0.79 (m, 6H). ¹³C NMR (CDCl₃, 126 MHz): δ 176.75, 168.32, 166.60, 133.02, 131.62, 129.86, 128.49, 76.55, 67.90, 67.80, 41.55, 40.86, 37.36, 36.76, 35.23, 33.22, 33.17, 32.77, 30.80, 27.58, 26.94, 24.38, 22.95, 16.42, 14.04, 11.81. HRMS(ESI): m/z calc. for C₂₆H₃₈O₆Cl [M+H]⁺: 481.2351, found: 481.2354.

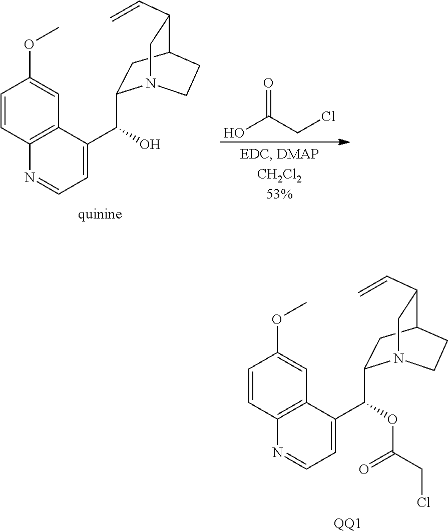

Procedure: To a solution of quinine (100 mg, 0.31 mmol) in dichloromethane (6 mL), chloroacetic acid (35 mg, 0.37 mmol), 1-Ethyl-3-(3-dimethylaminopropyl) carbodiimide hydrochloride (71 mg, 0.37 mmol), and dimethyl amino pyridine (4 mg, 0.031 mmol) were added and reaction was stirred for 24 hours then quenched by addition of water. The crude mixture was extracted with dichloromethane, and dry loaded onto silica by evaporation. Purification by flash chromatography (2% triethyl amine, 2% methanol in ethyl acetate) provided compound QQ1 (65.6 mg, 53%).

¹H NMR (CDCl₃, 500 MHz) δ 8.72 (d, J=4.5 Hz, 1H), 8.00 (d, J=9.2 Hz, 1H), 7.47-7.29 (m, 3H), 6.52 (d, J=7.2 Hz, 1H), 5.80 (ddd, J=17.0, 10.4, 7.4 Hz, 1H), 5.08-4.88 (m, 2H), 4.17-4.02 (m, 2H), 3.94 (s, 3H), 3.40 (q, J=8.0 Hz, 1H), 3.13-2.97 (m, 2H), 2.63 (dddd, J=27.5, 13.9, 5.0, 3.1 Hz, 2H), 2.27 (dddt, J=10.4, 5.9, 3.1, 1.5 Hz, 1H), 1.93-1.82 (m, 2H), 1.72 (dddd, J=10.5, 8.2, 6.5, 3.8 Hz, 1H), 1.60-1.45 (m, 2H). ¹³C NMR (CDCl₃, 126 MHz) δ 166.59, 158.20, 147.53, 144.94, 142.57, 141.63, 132.03, 126.95, 122.13, 119.03, 114.80, 101.36, 75.88, 59.14, 56.65, 55.84, 42.63, 40.96, 39.65, 27.76, 27.57, 24.41. HRMS(ESI): m/z calc. for C₂₂H₂₆N₂O₃Cl [M+H]⁺: 401.1632, found: 401.1624.

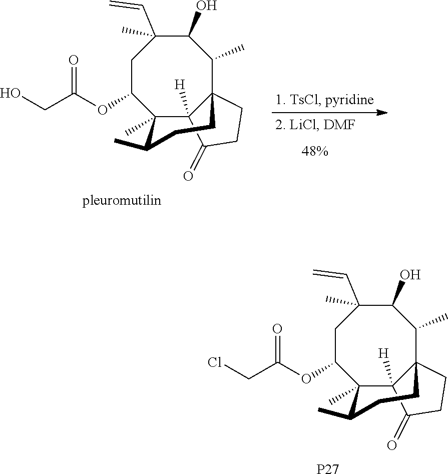

Procedure: To a solution containing Pleuromutilin (1 g, 2.6 mmole) in pyridine (8 mL) at 0° C. was added p-toluenesulfonyl chloride (590 mg, 3.1 mmole) in four portions over 15 minutes. The reaction was stirred at this temperature for 30 min and then stirred overnight at room temperature. Reaction was quenched by addition of cold water, extracted with ethyl acetate, and dried over sodium sulfate. The crude material was concentrated and dissolved in DMF (5 mL). To this solution was added LiCl (546 mg, 13 mmole). The reaction was then stirred overnight at 70° C. and quenched by addition of water. The crude reaction mixture was extracted with ethyl acetate, dried over sodium sulfate, and concentrated. Purification by flash chromatography (1:1 hexanes:ethyl acetate) afforded P27 (494 mg, 48% yield) as white solid.

¹H NMR (CDCl₃, 500 MHz) δ 6.53-6.44 (m, 1H), 5.78 (s, 1H), 5.37 (dd, J=11.0, 1.2 Hz, 1H), 5.22 (dd, J=17.4, 1.3 Hz, 1H), 3.98 (d, J=2.6 Hz, 2H), 3.37 (dd, J=10.6, 6.6 Hz, 1H), 2.33 (p, J=6.8 Hz, 1H), 2.29-2.16 (m, 2H), 2.12 (t, J=12.3 Hz, 2H), 1.83-1.74 (m, 1H), 1.74-1.62 (m, 2H), 1.61-1.37 (m, 7H), 1.34 (d, J=16.1 Hz, 1H), 1.19 (s, 3H), 1.13 (dd, J=14.1, 4.4 Hz, 1H), 0.89 (d, J=7.0 Hz, 3H), 0.74 (d, J=7.1 Hz, 3H). ¹³C NMR (CDCl₃, 126 MHz): δ 216.93, 166.13, 138.84, 117.55, 74.67, 70.69, 58.19, 45.54, 44.82, 44.10, 42.03, 41.64, 36.78, 36.12, 34.55, 30.50, 26.93, 26.46, 24.93, 16.87, 14.90, 11.63. HRMS(ESI): m/z calc. for C₂₂H₃₃ClO₄ [M+Na]⁺: 419.1960, found: 419.1965

Computational Analysis.

Molecular Property Distribution Violin Plots (FIG. 7)

Library data for approved cancer drugs was obtained from NCI as the Approve Oncology Drugs Set VIII (https://wiki.nci.nih.gov/display/NCIDTPdata/Compound+Sets). Library data for approved antibacterials was obtained from O'Shea and Moser (J. Med. Chem. 51, 2871-2878, (2008)). Drugbank library was obtained from the drugbank website (https://www.drugbank.ca/). Library data for the Chembridge-CL, Chembridge-EXP, and the MicroFormat libraries were obtained via the ChemBridge website (https://www.chembridge.com/). The Molecular Libraries Small Molecule Repository (MLSMR-NP) was obtained from the PubChem website (https://pubchem.ncbi.nlm.nih.gov/). PNAS Compound Collection (PNAS CC) was obtained from Clemons (Proc. Natl. Acad. Sci. 107, 18787-18792, (2010)). A detailed method for calculation of all the parameters can be found at the following site https://github.com/HergenrotherLab/ctd-pleuro.

### Example 36. Pharmaceutical Dosage Forms

The following formulations illustrate representative pharmaceutical dosage forms that may be used for the therapeutic or prophylactic administration of a compound of a formula described herein, a compound specifically disclosed herein, or a pharmaceutically acceptable salt or solvate thereof (hereinafter referred to as ‘Compound X’):

| (i) Tablet 1 | mg/tablet |
|---|---|
| ‘Composition X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| ‘Composition X’ | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| ‘Composition X’ | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/mL) | mg/mL |
|---|---|
| ‘Composition X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/mL) | mg/mL |
|---|---|
| ‘Composition X’ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |

| (vi) Aerosol | mg/can |
|---|---|
| ‘Composition X’ | 20 |
| Oleic acid | 10 |
| Trichloromonofluoromethane | 5,000 |
| Dichlorodifluoromethane | 10,000 |
| Dichlorotetrafluoroethane | 5,000 |

| (vii) Topical Gel 1 | wt. % |
|---|---|
| ‘Composition X’ | 5% |
| Carbomer 934 | 1.25% |
| Triethanolamine | q.s. |
| (pH adjustment to 5-7) | |
| Methyl paraben | 0.2% |
| Purified water | q.s. to 100 g |

| (viii) Topical Gel 2 | wt. % |
|---|---|
| ‘Composition X’ | 5% |
| Methylcellulose | 2% |
| Methyl paraben | 0.2% |
| Propyl paraben | 0.02% |
| Purified water | q.s. to 100 g |

| (ix) Topical Ointment | wt. % |
|---|---|
| ‘Composition X’ | 5% |
| Propylene glycol | 1% |
| Anhydrous ointment base | 40% |
| Polysorbate 80 | 2% |
| Methyl paraben | 0.2% |
| Purified water | q.s. to 100 g |

| (x) Topical Cream 1 | wt. % |
|---|---|
| ‘Composition X’ | 5% |
| White bees wax | 10% |
| Liquid paraffin | 30% |
| Benzyl alcohol | 5% |
| Purified water | q.s. to 100 g |

| (xi) Topical Cream 2 | wt. % |
|---|---|
| ‘Composition X’ | 5% |
| Stearic acid | 10% |
| Glyceryl monostearate | 3% |
| Polyoxyethylene stearyl ether | 3% |
| Sorbitol | 5% |
| Isopropyl palmitate | 2% |
| Methyl Paraben | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Compound X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A compound of Formula I: 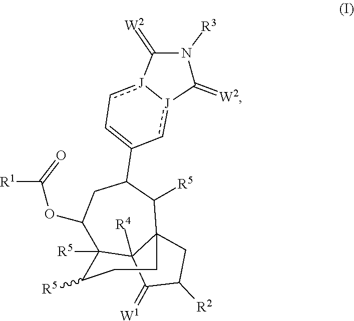
or a stereoisomer or salt thereof;
wherein
J is N or CH and [symbol] is a single bond, or C and [symbol] is a double bond;
R¹ is —(C₁-C₆)alkyl-X, —(C₂-C₆)alkenyl-X, halo, OH, or heteroaryl, wherein the (C₁-C₆)alkyl moiety of —(C₁-C₆)alkyl-X is substituted optionally with halo;
X is halo, OH, or —O(C═O)CH₃, or absent;
R² and R⁴ are independently OR^{A}, H, or halo, wherein R^{A} is H, —(C₁-C₆)alkyl, or —(C═O)CH₃;
R³ is aryl, heteroaryl, heterocycloalkyl, —(C₃-C₆)cycloalkyl, —(C₂-C₆)alkynyl, or a group comprising a fluorescent tag, wherein aryl or heteroaryl is substituted optionally with halo, OH or —(C₁-C₆)alkyl;
each R⁵ is independently CH₃, —(C₂-C₆)alkyl or H;
W¹ is O, S, or absent; and
each W² is independently O, S, or absent.

2. The compound of claim 1 wherein J is N and [symbol] is a single bond.

3. The compound of claim 1 wherein J is CH and [symbol] is a single bond.

4. The compound of claim 1 wherein R¹ is —(C₁-C₆)alkyl-X.

5. The compound of claim 4 wherein R¹ is —CH₂Cl, —CH₂F, —CH₂I, —CH₃, —CH₂O(C═O)CH₃, —CHCl₂, vinyl, allyl, ethynyl, propynyl, or 2-furanyl.

6. The compound of claim 1 wherein R² and R⁴ are OR^{A}.

7. The compound of claim 1 wherein R³ is aryl or —(C₂-C₆)alkynyl.

8. The compound of claim 7 wherein R³ is phenyl or propynyl.

9. The compound of claim 1 wherein W¹ and W² are O.

10. The compound of claim 9 wherein R⁴ is OH and R⁵ is CH₃.

11. The compound of claim 10 wherein R³ is phenyl, J is N, and [symbol] is a single bond.

12. The compound of claim 1 wherein a compound of Formula I is a compound of Formula II, III, or IV: 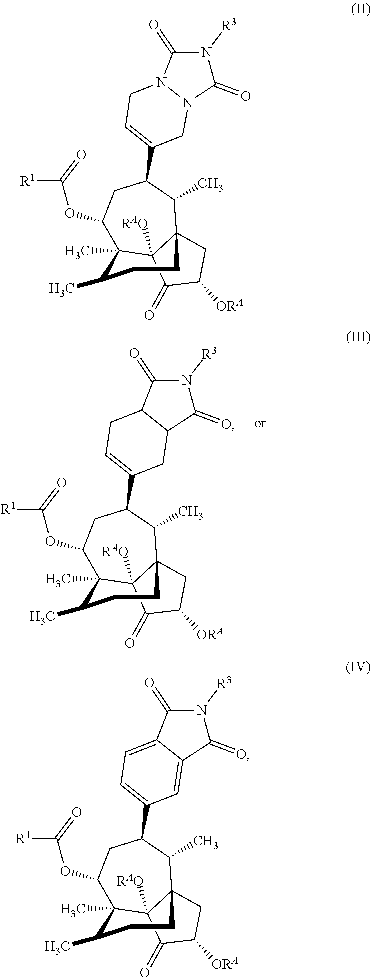
wherein
R¹ is —CH₃, —CH₂F, —CH₂Cl, —CH₂I, —CH₂O(C═O)CH₃, —CHCl₂, vinyl, allyl, ethynyl, propynyl, or 2-furanyl; and
each R^{A} is independently H, —(C₁-C₆)alkyl, or —(C═O)CH₃.

13. The compound of claim 12 wherein R³ is phenyl, propynyl, or a group comprising a fluorescent tag.

14. The compound of claim 1 wherein the compound of Formula I is:

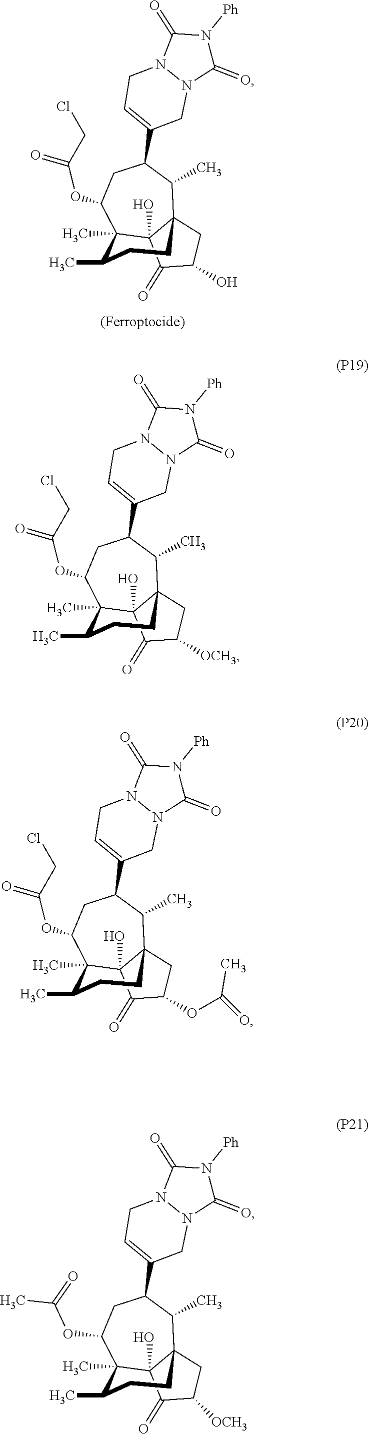

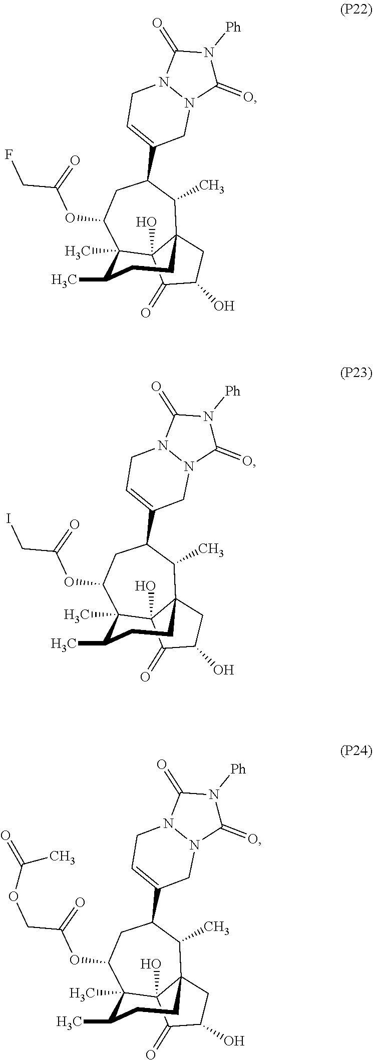

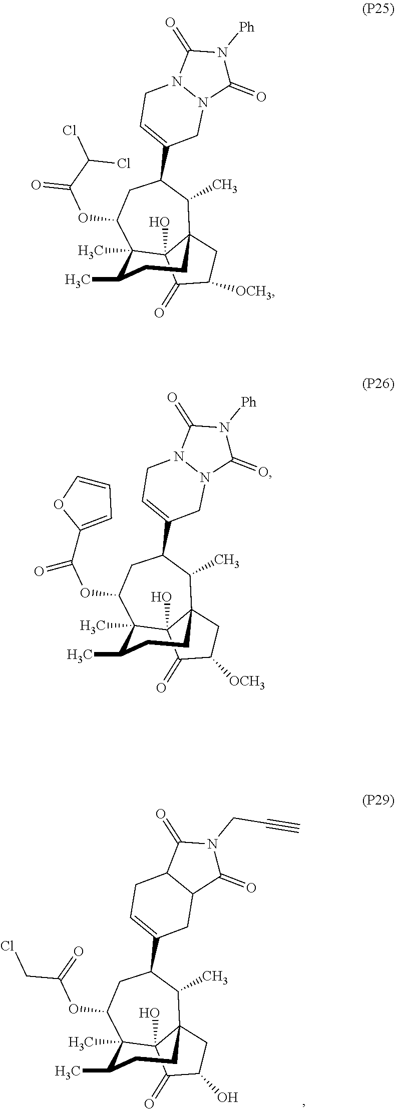

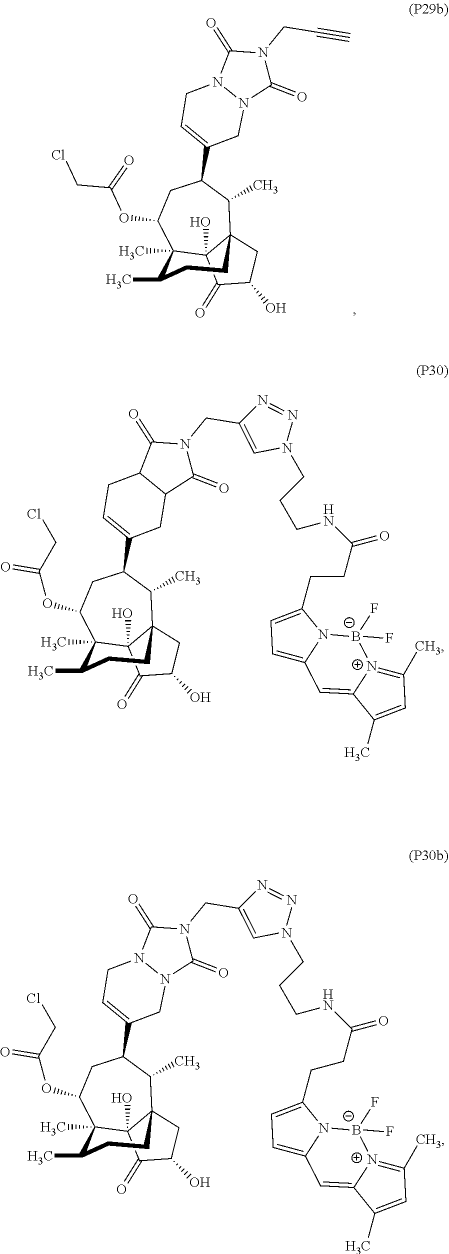

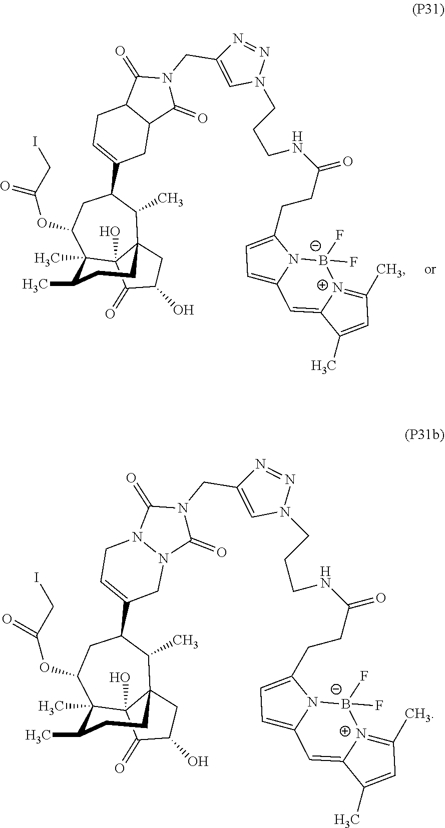

15. The compound of claim 14 wherein the compound is Ferroptocide.

16. A composition comprising a compound of claim 1 and a pharmaceutically acceptable buffer, carrier, diluent, or excipient.

17. A method for inducing ferroptosis in cancer cells comprising contacting the cancer cell with an effective amount of a compound of claim 1, thereby inducing ferroptosis.

18. A method for treating cancer in a cancer subject comprising administering an effective amount of a compound of claim 1 to the cancer subject in need of cancer treatment wherein the cancer is thereby treated.

19. The method of claim 18 wherein the cancer is blood cancer, brain cancer, breast cancer, colorectal cancer, liver cancer, lung cancer, ovarian cancer, pancreatic cancer, prostate cancer, or skin cancer.

20. The method of claim 18 wherein the compound is Ferroptocide.
